(19) United States
(12) Patent Application Publication
(10) Pub. No.: US 2026/0272892 A1
(43) Pub. Date: Sep. 17, 2026

(54) Mitogen-Activated Protein Kinase Kinase 1/2 (MEK1/2) Degraders Combined with BCL-2 Inhibitors for the Treatment of Cancer

(71) Applicant: Institute For Cancer Research d/b/a The Research Institute of Fox Chase Cancer Center, Philadelphia, PA (US)

(72) Inventors: James S. Duncan, Huntingdon Valley, PA (US); Jason Wasserman, Willow Grove, PA (US); Glenn Doyle, Drexel Hill, PA (US); Alison M. Kurimchak, Philadelphia, PA (US)

(21) Appl. No.: 19/563,568

(22) Filed: Mar. 11, 2026

Related U.S. Application Data
(60) Provisional application No. 63/770,305, filed on Mar. 11, 2025.

(51) Int. Cl. A61K 31/427; A61K 31/21; A61K 31/404; A61K 31/635; A61K 31/7105; A61K 39/00; A61P 35/00; C12N 15/113

(52) U.S. Cl. CPC A61K 31/427; A61K 31/21; A61K 31/404; A61K 31/635; A61K 31/7105; A61K 39/0011; A61P 35/00; C12N 15/113; C12N 2310/11

(57) ABSTRACT

Compositions and methods of treating cancer by administering a Mitogen-Activated Protein Kinase Kinases 1 and 2 (MEK1/2) degrader and a BCL-2 inhibitor to a subject in need thereof.

### CROSS-REFERENCE TO RELATED APPLICATIONS

This application claims benefit of the filing date of U.S. Provisional Application No. 63/770,305, filed Mar. 11, 2025. The content of this application is hereby incorporated herein by reference in its entirety.

### STATEMENT REGARDING FEDERALLY FUNDED RESEARCH

This invention was made with government support under CA211670 and CA282766 awarded by the National Institutes of Health. The government has certain rights in the invention.

### INCORPORATION OF THE SEQUENCE LISTING

The present application contains a sequence listing that is submitted concurrent with the filing of this application, containing the file name “38235_0096U2_SL.xml” which is 28,672 bytes in size, created on Mar. 10, 2026, and is herein incorporated by reference in its entirety.

### FIELD

The present disclosure is directed, in part, to treating cancer in a subject in need thereof by administering a dual RAF/MEK degrader to the subject.

### BACKGROUND

Activating K-ras (KRAS) mutations frequently occur in several cancers including the majority of pancreatic ductal adenocarcinoma (96%), about 50% of colorectal cancers, about 30% of lung adenocarcinoma, and about 20% of endometrial carcinomas (Munoz-Maldonado et al., Front. Oncol., 2019, 9, 1088). It has been shown that mutant KRAS preferentially signals through RAF-MEK-ERK signaling and that blockade of this pathway is important for tumor regression (Lito et al., Cancer Cell, 2014, 25, 697-710). However, targeted MEK inhibitors (MEKi) have shown limited clinical benefit in the treatment of KRAS mutant cancers due to resistance caused by the feedback activation of the upstream kinase CRAF (RAF1). Consequently, there are no single agent MEKi trials ongoing for KRAS mutant cancers, with clinical trials focusing on combinations of CRAF and MEK inhibitors. Although genetic depletion of CRAF has been shown to cause tumor regression in KRAS mutant cancer models, CRAF inhibitors have shown limited efficacy for cancer treatment (Venkatanarayan et al., Cell Reports, 2022, 38, 10351). It has been shown that depletion of CRAF leads to apoptosis in KRAS mutant cancers independent of MEK1/2 activity, where CRAF promotes suppression of apoptosis and de-differentiation via kinase-independent functions (McCormick et al., Cancer Cell, 2018, 33, 158-159). Thus, the development of CRAF protein degraders that can block both kinase-dependent and independent functions of CRAF are needed for the treatment of RAS-altered cancers.

### SUMMARY

The present disclosure provides methods of treating a human disease that requires RAF-MEK in a subject in need thereof, the method comprising administering a dual RAF/MEK degrader to the subject.

The present disclosure also provides dual RAF/MEK degraders for use in treating a human disease that requires RAF-MEK.

Disclosed herein are methods of treating a human disease that requires RAF-Mitogen-Activated Protein Kinase Kinase (RAF-MEK) in a subject in need thereof, the methods comprising administering a dual RAF/MEK degrader to the subject.

Disclosed herein are dual RAF/Mitogen-Activated Protein Kinase Kinase (RAF/MEK) degraders for use in treating a human disease that requires RAF-Mitogen-Activated Protein Kinase Kinase (RAF-MEK).

Disclosed herein are compositions comprising RAF/Mitogen-Activated Protein Kinase Kinase (RAF/MEK) degraders combined with BCL-2 inhibitors.

Disclosed herein are methods of treating cancer in a subject, the methods comprising administering to a subject in need thereof, a RAF/Mitogen-Activated Protein Kinase Kinase (RAF/MEK) degrader and a BCL-2 inhibitor.

Disclosed herein are methods of treating a cancer in a subject, the methods comprising administering a therapeutically effective amount of a MEK1/2 degrader and a therapeutically effective amount of a BCL-2 inhibitor to a subject in need thereof, thereby treating the cancer in the subject.

Disclosed herein are methods of reducing tumor growth in a subject, the methods comprising administering a therapeutically effective amount of a MEK1/2 degrader and a therapeutically effective amount of a BCL-2 inhibitor to a subject in need thereof, thereby reducing the tumor growth in the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-B show that MS934-treatment of KRAS mutant pancreatic cells reduces MEK1, MEK2, and CRAF protein levels. FIG. 1A shows PANC-1 cells treated with escalating doses of PD0325091 (0, 0.0001, 0.001 0.014, 0.041, 0.123, 0.370, 1 μM) for 24 hours, and MAPK signaling components determined by immunoblotting; blots that are representative of 3 independent blots. FIG. 1B shows PANC-1 cells treated with escalating doses of MS934 (0, 0.014, 0.041, 0.123, 0.370, 1.1, 3.3, or 10 μM) for 24 hours, and MAPK signaling components determined by immunoblotting; blots are representative of 3 independent blots. FIG. 1C shows densitometric analysis of the MAPK protein immunoblots using ImageJ as means±SD from 3 blots, each normalized to loading control, GAPDH; DC₅₀ values were determined in Prism Software by quantitating the dose of MS934 that reduces MAPK signaling protein levels 50% of the DMSO control treatment.

FIGS. 2A-J show CRAF protein is reduced by MS934-treatment across cancer cell lines, cancer cell lines treated with escalating doses of MS934 (each 0, 0.02, 0.05, 0.10, 0.20, or 0.50 μM) for 24 hours, and MAPK signaling components determined by immunoblotting (FIGS. 2A, 2B, 2C, and 2D); cancer cell lines treated with escalating doses of MS934 (each 0, 0.014, 0.041, 0.123, 0.370, 1.1, 3.3, or 10 μM) for 24 hours, and MAPK signaling components determined by immunoblotting (FIGS. 2E, 2F, 2G, 2H, and 2I); and bar plot representing the DC₅₀ values for CRAF across cell lines treated with MS934; DC₅₀ values were determined in Prism Software by quantitating the dose of MS934 that reduces MAPK signaling protein levels 50% of the DMSO control treatment; significance determined by paired t-test using Prism software; ***P<0.001 (FIG. 2J).

FIGS. 3A-F show collateral degradation of CRAF by MS934 through a VHL-proteasome dependent mechanism in PANC-1 cells treated with control or VHL siRNAs for 48 hours and then treated with DMSO or 1 μM MS934 for 24 hours, and MAPK signaling components determined by immunoblotting; blots are representative of 3 independent blots (FIG. 3A); densitometric analysis of the MAPK protein immunoblots, using ImageJ as means±SD from 3 blots, each normalized to loading control, GAPDH (FIG. 3B); PANC-1 cells treated with 1 μM MS934 and 1 μM PD0325901 or 100 nM bortezomib for 24 hours, and CRAF, MEK1 and MEK2 levels determined by immunoblotting; blots are representative of 3 independent blots (FIG. 3C); densitometric analysis of the MAPK protein immunoblots, using ImageJ as means±SD from 3 blots, each normalized to loading control, GAPDH (FIG. 3D); TUBE1 pulldown for ubiquitinated CRAF in PANC-1 cells treated with DMSO, MS934 (1 μM), bortezomib (0.1 μM) or the combination for 24 hours; blots are representative of 3 independent blots (FIG. 3E); and densitometric analysis of the CRAF levels using ImageJ as means±SD from 3 blots, each normalized to loading control, GAPDH (FIG. 3F).

FIGS. 4A-H show feedback induced CRAF-MEK dimerization by MS934-treatment promotes collateral degradation of CRAF, MEK pulldown in PANC-1 cells treated with DMSO or PD0325901 (0.1 μM) for 3 hours and CRAF binding to MEK1 determined by immunoblotting; blots are representative of 3 independent blots (FIG. 4A); densitometric analysis of CRAF protein levels using ImageJ as means±SD from 3 blots, each normalized to loading control, GAPDH (FIG. 4B); PANC-1 cells treated with control or MEK1/2 siRNAs for 48 hours followed by MS934 (0.1 μM) treatment for 24 hours and MAPK signaling components determined by immunoblotting; blots are representative of 3 independent blots (FIG. 4C); densitometric analysis of the CRAF protein levels using ImageJ as means±SD from 3 blots, each normalized to loading control, GAPDH (FIG. 4D); PANC-1 cells treated with DMSO, MS934 (0.1 μM), RAF709 (1 μM), or the combination for 24 hours and MAPK signaling proteins determined by immunoblotting; blots are representative of 3 independent blots (FIG. 4E); densitometric analysis of CRAF, MEK1 or MEK2 protein levels using ImageJ as means±SD from 3 blots, each normalized to loading control, GAPDH (FIGS. 4F and 4G).

FIGS. 5A-C show CRAF/MEK degradation reduces PLK1 and Rho GTPase signaling in MEK inhibitor-resistant KRAS mutant pancreatic cancer cells, circos plot of signaling pathways reduced in PANC-1 cells following 24 hours of treatment by PD0325901 (0.1 μM) or MS934 (10 μM) (FIG. 5A); hierarchical clustering of pathways downregulated by PD0325901 (0.1 μM) or MS934 (10 μM) treatment PANC-1 cells; heat map color depicts P values for pathway enrichment (FIG. 5B); and hierarchical clustering of predicted transcription factors reduced by PD0325901 (0.1 μM) or MS934 (10 μM) treatment for 24 hours in PANC-1 cells; heat map color depicts P values for pathway enrichment; proteins repressed by PD0325901 or MS934 treatment (FDR<0.05) were imported into Metascape pathway analysis (FIG. 5C).

FIG. 6 shows CRAF/MEK degradation reduces growth of KRAS mutant cell lines; cell lines harboring KRAS mutations were treated with escalating doses of MS934 (each 0, 0.014, 0.041, 0.123, 0.370, 1.1, 3.3, or 10 μM) for 14 days and colony formation assessed by crystal violet staining; images are representative of three independent assays.

FIGS. 7A-C show ARAF, BRAF and CRAF protein levels reduced at higher concentrations of MS934; immunoblot of HT-29 (FIG. 7A), HCT 116 (FIG. 7B), or NCI-H23 (FIG. 7C) cells treated with escalating doses of MS934 for 24 hours.

FIGS. 8A-M show that MEK1/2 degradation but not inhibition promotes CRAF loss. FIGS. 8A-B show a Volcano plot of proteins increased or reduced in abundance in PANC-1 cells treated with PD0325901 (FIG. 8A) or MS934 (FIG. 8B) for 24 hours relative to DMSO-treated cells. Differences in protein log 2 LFQ intensities among treated or control cells were determined by paired t-test permutation-based adjusted P values at FDR of <0.05 using Perseus software. FIGS. 8C-D show immunoblotting for MAPK signaling components in PANC-1 cells treated with escalating doses of PD0325901, MS934, or MS934(−) for 24 hours. Blots are representative, and densitometric analysis of CRAF (FIG. 8D) are means 1 SEM from three blots, each normalized to the loading control, GAPDH. FIG. 8E shows ARAF, BRAF, RAF1 mRNA levels in PANC-1 cells following treatment with 1 μM MS934 for 24 hours, as determined by qRT-PCR. Data are means 1 SEM of three independent experiments. FIG. 8F show immunoblotting for CRAF, MEK1/2 or ERK1/2 in PANC-1 cells treated with DMSO, 0.1 μM MS934, 0.1 μM bortezomib, or the combination for 24 hours. Cells were treated with bortezomib 2 hours prior to administering MS934. Blots are representative, and densitometric analysis of CRAF protein levels are means±SEM from three blots, each normalized to the loading control, GAPDH. FIG. 8G show immunoblotting for MAPK signaling components in non-tumorigenic cell line MCF-10A treated with escalating doses of PD0325901, or MS934 for 24 hours. FIG. 8H shows immunoblotting for MAPK signaling components in A549 cells treated with 1 mM MS934, MEK1/2 inhibitors, or ERK1/2 inhibitor for 24 hours. FIGS. 8I-J show microscopy image (FIG. 8I) and immunoblot analysis (FIG. 8J) of mCherry-CRAF or MEK1-GFP expression in PANC-1 cells treated with 1 μM MS934 for 24 hours. Images are representative of three independent experiments. Scale bars, 200 μm. FIG. 8K show immunoblot analysis for mCherry-CRAF and MEK1-GFP in PANC-1 cells treated with DMSO, 1 μM MS934, 0.1 μM bortezomib, or the combination for 24 hours. Cells were treated with bortezomib 2 hours prior to administering MS934. FIGS. 8L-M show immunoblot analysis of MAPK signaling components in Capan-1 cells treated with escalating doses of MS432 (FIG. 8H) or MS910 (FIG. 8I) for 24 hours.

FIGS. 9A-N show that CRAF protein stability is dependent on MEK1/2 expression in cells. FIGS. 9A-B show immunoblot analysis of MAPK signaling in HCT-116, A549, PANC-1, and NCI-H23 following 1 mM MS934 treatment across a 72-hour time course. Fresh MS934 was administered every 24 hours. Blots are representative, and densitometric analysis of MEK1, MEK2, and CRAF (FIG. 9B) are means±SEM across cell line blots, each normalized to the loading control, GAPDH. FIGS. 9C-G show the immunoprecipitation (IP) of ubiquitin-modified proteins using TUBE1 beads from PANC-1 cells co-expressing mCherry-CRAF and MEK1-GFP (FIGS. 9C-E) or HCT-116 cells (FIGS. 9F-G) treated with 1 mM MS934 for 1, 4 or 8 hours. The TUBE1 IP elutes were blotted with indicated antibodies. FIG. 9H shows the immunoblot analysis of RAF and MEK1/2 protein levels in HCT-116 cells treated with control siRNAs or MEK1, MEK2, or MEK1 and MEK2 siRNAs and cultured for 48 hours. FIG. 9I shows the immunoblot analysis of RAF and MEK1/2 levels in PANC-1, NCI-H23, HCT-116 and A549 treated with 1 mM MS934 or MEK1 and MEK2 siRNAs (siMEK1/2). FIG. 9J shows the immunoblot analysis of RAF and MEK1/2 levels in HCT-116 cells treated with siRNAs targeting both MEK1 and MEK2 for 8, 24, 48, and 72 hours. FIG. 9K shows the immunoblotting for CRAF, MEK1/2, or ERK1/2 in PANC-1 cells treated with DMSO, 1 μM PD0325901, 0.1 μM MS934, or the combination for 24 hours. Cells were treated with PD0325901 2 hours prior to adding MS934. Blots are representative, and densitometric analysis of CRAF levels are means±SEM from three blots, each normalized to the loading control, GAPDH. FIG. 9L shows the immunoblot analysis for CRAF, MEK1/2 or ERK1/2 in PANC-1 cells transfected with control siRNA or siRNAs targeting VHL and treated with DMSO or 0.1 μM MS934 for 24 hours. Blots are representative, and densitometric analysis of CRAF protein levels are means±SEM from three blots, each normalized to the loading control, GAPDH. FIGS. 9M-N show the immunoblot analysis of MAPK signaling in PANC-1 cells treated with a single dose of 0.1 mM MS934 (FIG. 9M) or PD0325901 (FIG. 9N) and followed across a 96-hour period. No drug replenishment was performed.

FIGS. 10A-O show MEK1/2 degradation or knockdown caused rapid de-phosphorylation of CRAF but not ARAF or BRAF, promoting protein degradation. FIGS. 10A-B show the immunoblot analysis of CRAF phosphorylation in HCT-116, A549, PANC-1, and NCI-H23 following 1 mM MS934 treatment across a 72-hour time course. Fresh MS934 was administered every 24 hours. Blots are representative, and densitometric analysis of CRAF total, p-S259, p-S289/296/301, p-S338, and p-S621 (FIG. 10B) are means±SEM across cell line blots, each normalized to the loading control, GAPDH. CRAF, MEK1, and MEK2 blots are also shown in FIG. 9A. FIG. 10C shows the immunoblotting for CRAF phosphorylation in HCT-116 cells treated with 1 mM PD0325901, MS934, or the combination for 0, 0.5, 1, 2, 3, 4, or 24 hours. FIG. 10D shows the immunoblot analysis of CRAF phosphorylation levels in HCT-116 cells treated with non-targeting siRNAs or MEK1, MEK2, or MEK1 and MEK2 siRNAs and cultured for 48 hours. FIG. 10E shows the immunoblot analysis of CRAF phosphorylation levels in HCT-116 cells treated with siRNAs targeting both MEK1 and MEK2 for 8, 24, 48, and 72 hours. FIGS. 10F-G show the immunoblotting of CRAF phosphorylation at S621 levels in HCT-116 cells treated with 1 mM MS934 alone or in combination with 2.5 nM Calyculin A for 0, 1, or 2 hours. Cells were pre-treated with Calyculin A for 30 minutes followed by MS934 treatment for indicated times. Blots are representative, and densitometric analysis of CRAF p-S621 (FIG. 10G) are means±SD of 3 independent blots, each normalized to total CRAF protein and the loading control, GAPDH. FIGS. 10H-I show the immunoprecipitation (IP) of endogenous CRAF from HCT-116 cells treated with 1 mM PD0325901 (FIG. 10H) or 1 mM MS934 for 0, 4, or 24 hours. The CRAF IPs were blotted with the indicated antibodies. Input samples were used as controls. FIGS. 10J-L show the immunoblot analysis of RAF phosphorylation in PANC-1 cells expressing Flag-CRAF (FIG. 10J), Flag-BRAF (FIG. 10K), or Flag-ARAF (FIG. 10L) treated with 0, 0.1, and 1 mM MS934 for 24 hours. FIGS. 10M-O show the immunoprecipitation (IP) of endogenous CRAF (FIG. 10M), BRAF (FIG. 10N), or ARAF (FIG. 10O) from HCT-116 cells treated with 1 mM MS934 for 0, 4, or 24 hours. The RAF IPs were blotted with the indicated antibodies. Input samples were used as controls.

FIGS. 11A-H show that MEK1/2-binding to CRAF and not catalytic activity promotes CRAF stabilization and activation. FIG. 11A show the immunoblot analysis of total and phosphorylated Flag-CRAF levels in PANC-1 cells transfected with Flag-CRAF (2.5 μg) and escalating concentrations of Myc-MEK1 (37.5, 75 ng), Myc-MEK2 (50, 100 ng) or the combination of Myc-MEK1 and Myc-MEK2 (halving amounts) for 48 hrs. FIGS. 11B-C show the immunoblot analysis of total and phosphorylated Flag-CRAF levels in PANC-1 cells transfected with Flag-CRAF or Flag-CRAF(S471A) (2.5 μg) and Myc-MEK1 (37.5 ng)/Myc-MEK2 (25 ng). Blots are representative, and densitometric analysis of p-S621 and CRAF (FIG. 11C) are means+SD from three blots, each normalized to the loading control, GAPDH. FIG. 11D show the immunoblot analysis of total and phosphorylated Flag-CRAF levels in PANC-1 cells transfected with Flag-CRAF and Myc-MEK1/2 wild-type of Myc-MEK1(K97M)/MycMEK2(K101M) (25 ng per). FIGS. 11E-F show the immunoblotting of total and phosphorylated Flag-CRAF levels in MS934-treated (1 μM) PANC-1 cells transfected with Flag-CRAF (2.5 μg) and escalating concentrations of Myc-MEK1(K97M) (75, 150, 300 ng) (FIG. 11E) or Myc-MEK2(K101M) (75, 150, 300 ng) (FIG. 11F) for 48 hrs. Cells were pre-treated with MS934 for 4 hours to deplete endogenous MEK1/2. FIGS. 11G-H show the immunoblot analysis of endogenous CRAF total and p-S621 levels in PANC-1 cells expressing si-MEK1/2 #1 resistant MEK1-GFP(WT) or MEK1-GFP(IRI) treated with control, si-MEK1/2 #1 or MEK1/2 #2 siRNAs for 72 hours. Blots are representative, and densitometric analysis of p-S621 and CRAF (FIG. 11G) are means+SEM from three blots, each normalized to the loading control, GAPDH.

FIGS. 12A-I show that MEK1/2 is required for stabilizing mature CRAF but not the immature form. FIG. 12A shows the immunoblot analysis of CRAF protein levels in HCT-116 cells treated with MEK1/2 siRNAs for 72 hours or 1 mM MS934 for 24 hours. FIG. 12B shows the immunoblotting for CRAF total and p-S621 levels in HCT-116 cells treated with DMSO, 1 mM MS934, 100 mg/ml cycloheximide, 0.05 mM STA9090, or the combination of MS934 and cycloheximide. FIG. 12C shows the immunoblot analysis of RAF levels in HCT-116 cells treated with DMSO, 1 mM MS934, 0.05 mM STA9090, or the combination for 24 h. FIG. 12D shows the immunoblot analysis for p-S621 and CRAF levels in HCT-116 cells treated with DMSO or escalating doses of MS934 in the presence or absence of 2 mM RAD001 for 24 hours. FIG. 12E shows the immunoblotting for Flag-CRAF levels in PANC-1 cells transfected with Flag-CRAF(WT) or Flag-CRAF(S621A) and cells expressing Flag-CRAF(WT) were treated with DMSO, 0.1 or 1 mM MS934. FIGS. 12F-H show the immunoblot analysis of Flag-CRAF levels in PANC-1 cells stably expressing MEK1-GFP transfected with Flag-CRAF(WT) or Flag-CRAF(S621A) (FIG. 12F) or Flag-CRAF(WT) or Flag-CRAF(K375M) (FIG. 12G) or Flag-CRAF(S471A) treated with DMSO, 1 mM MS934, 100 mg/ml cycloheximide, or the combination of MS934 and cycloheximide for 24 hours. FIG. 12I show the immunoblotting for Flag-CRAF levels in PANC-1 cells stably expressing MEK1-GFP transfected with Flag-CRAF(WT), Flag-CRAF(S621A), Flag-CRAF(S471A) or Flag-CRAF(K375M) treated with DMSO, or 0.05 mM STA9090 for 24 hours.

FIGS. 13A-I show that immature CRAF has anti-apoptotic functions outside the MAPK signaling pathway. FIGS. 13A-B shows the immunoblot analysis of cleaved PARP levels in HCT-116 (FIG. 13A) or A549 (FIG. 13B) cells treated with DMSO or 1 mM MS934 or upon short hairpin RNA (shRNA)-mediated depletion of CRAF (shCRAF) or non-targeting shRNA (shNT) (1 mg/ml doxycycline for 120 h (FIG. 13A) or 72 h (FIG. 13B)). FIGS. 13C-D show the immunoblot analysis of cleaved PARP levels in HCT-116 (FIG. 13C) or A549 (FIG. 13D) cells treated with DMSO or 1 mM MS934 for 24 h followed by short hairpin RNA (shRNA)-mediated depletion of CRAF (shCRAF) or non-targeting shRNA (shNT) (1 mg/ml doxycycline for 96 h (FIG. 13C) or 120 h (FIG. 13D)). FIG. 13E shows the immunoprecipitation (IP) of Flag-CRAF from PANC-1 cells stably expressing MEK1-GFP treated with DMSO or 1 mM MS934 for 24 h or IP of Flag-CRAF(K375M). The Flag-CRAF IPs were blotted with the indicated antibodies. IP of Flag-GFP and input samples were used as controls. FIGS. 13F-G show the Caspase 3/7 activation analysis in HCT-116 (FIG. 13F) or A549 (FIG. 13G) cells treated with DMSO, 0.005 mM PD0325901, 0.5 mM MS934, 1 mM Navitoclax, or the combination of PD0325901 or MS934 and Navitoclax for 24 h. Data are means 1 SEM of three independent experiments. FIGS. 13H-I show the soft-agar colony formation assay in HCT-116 (FIG. 13H) or A549 (FIG. 13I) cells treated with DMSO, 0.005 mM PD0325901, 0.5 mM MS934, 1 mM Navitoclax, or the combination of PD0325901 or MS934 and Navitoclax for 2 weeks. Data are means±SEM of three independent experiments.

FIGS. 14A-G show a stable complex of CRAF-MEK1/2-HSP90/CDC37-14-3-3 exists that is primed for activation by RAS. FIG. 14A shows immunoprecipitation (IP) of Flag-tagged CRAF constructs from PANC-1 cells stably expressing MEK1-GFP. The Flag-CRAF IPs were blotted with the indicated antibodies. IP of Flag-GFP and input samples were used as controls. FIG. 14B show the immunoblot analysis of Flag-tagged CRAF constructs in PANC-1 cells stably expressing MEK1-GFP transfected with Flag-CRAF(WT), Flag-CRAF(R401H) or Flag-CRAF(R89L) treated with DMSO, 1 mM MS934, 100 mg/ml cycloheximide, or the combination of MS934 and cycloheximide for 24 hours. FIGS. 14C-E show the immunoblot analysis of CRAF signaling in PANC-1 cells co-transfected with Myc-MEK1/Myc-MEK2 and Flag-CRAF(WT), Flag-CRAF(R89L), Flag-CRAF(R401H) or Flag-CRAF(K375M) for 48 hrs. Blots are representative, and densitometric analysis of p-MEK1/2 and (FIGS. 14D/E) are means±1 SEM from three blots, each normalized to the loading control, GAPDH, Myc tag and Flag levels. FIG. 14F shows the immunoprecipitation (IP) of endogenous MEK1 from HCT-116 cells treated with 1 mM ERK1/2 inhibitor SCH772984 for 24 hours. The MEK1 IP was blotted for CRAF and MEK1 proteins. Blots are representative, and densitometric analysis of CRAF are means±SEM from three blots, each normalized to the loading control, GAPDH. FIG. 14G shows a model of MEK1/2 regulation of CRAF stabilization, maturation, and activation.

FIGS. 15A-H show that MEK1/2 degradation causes downregulation of CRAF protein levels. FIG. 15A show the principal component analysis (PCA) of global proteome responses in PANC-1 cells treated with DMSO, or 1 mM PD0325901 or MS934 for 24 h as determined by single-run proteome analysis. FIGS. 15B-E show the immunoblot analysis of MAPK signaling in KRAS mutant cell lines (FIG. 15B), NF1-deficient (FIG. 15C), BRAF mutant (FIG. 15D), or non-tumorigenic cell line (FIG. 15E) treated with escalating doses of MS934 for 24 h. FIG. 15F shows immunoblotting for CRAF, MEK1/2, or ERK1/2 in A549 treated with DMSO, 0.1 μM MS934, 0.1 μM bortezomib, or the combination for 24 hours. Cells were treated with bortezomib 2 hours prior to administering MS934. FIG. 15G show ARAF, BRAF, RAF1 mRNA levels in HCT-116 cells following treatment with escalating doses of MS934 for 24 hours, as determined by qRT-PCR. Data are means±SEM of three independent experiments. FIG. 15H show immunoblot analysis of MYC-CRAF levels in PANC-1 cells stably expressing MEK1-GFP treated with escalating doses of MS934 for 24 h.

FIGS. 16A-I show that MEK1/2 degradation and knockdown reduce CRAF protein expression. FIG. 16A shows that the Volcano plot of proteins increased or reduced in abundance in PANC-1 cells treated with MS934 for 4 hours relative to DMSO-treated cells. Differences in protein log 2 LFQ intensities among treated or control cells were determined by paired t-test permutation-based adjusted P values at FDR of <0.05 using Perseus software. FIG. 16B shows the differences in raw LFQ values for MEK1, MEK2, and CRAF in response to DMSO or MS934 treatment for 4 or 24 hours. FIG. 16C shows the immunoblot analysis of RAF and MEK1/2 protein levels in A549 cells treated with control siRNAs or MEK1, MEK2, or MEK1 and MEK2 siRNAs and cultured for 48 hours. FIGS. 16D-E show the immunoblot analysis of CRAF levels in A549 cells treated with non-targeting siRNAs or siRNAs targeting CRAF (FIG. 16A) or MEK1/2, ERK1/2, or KSR1/2 for 72 h. FIG. 16F shows MEK1/2, ARAF, BRAF, RAF1 mRNA levels in HCT-116 cells following treatment with non-targeting siRNAs or siRNAs targeting MEK1 and MEK2 for 72 hours, as determined by qRT-PCR. Data are means 1 SD of three independent experiments. FIG. 16G shows immunoblotting for CRAF, MEK1/2, or ERK1/2 in NCI-H23 cells treated with DMSO, 1 μM PD0325901, 0.1 μM MS934, or the combination for 24 hours. Cells were treated with PD0325901 2 hours prior to adding MS934. Blots are representative, and densitometric analysis of CRAF levels are means±SEM from three blots, each normalized to the loading control, GAPDH. FIG. 16H shows the immunoblot analysis for CRAF, MEK1/2, or ERK1/2 in NCI-H23 cells transfected with non-targeting siRNA or siRNAs targeting VHL and treated with DMSO or 0.1 μM MS934 for 24 hours. Blots are representative, and densitometric analysis of CRAF protein levels are means±SD from three blots, each normalized to the loading control, GAPDH. FIG. 16I shows the immunoblot analysis for CRAF, or MEK1/2 in HCT-116 cells transfected with non-targeting siRNA or siRNAs targeting CUL2 and treated with DMSO or 0.1 μM MS934 for 24 hours.

FIGS. 17A-H show that MEK1/2 is important for preserving the phosphorylation of CRAF preventing its degradation. FIG. 17A shows the immunoblot analysis of CRAF phosphorylation in HEK293T cells treated with 1 mM MS934 for 4 or 24 hours. FIG. 17B shows the immunoblotting for CRAF phosphorylation in HCT-116 cells treated with non-targeting siRNAs, siRNAs targeting a shared sequence amongst MEK1/2, siRNAs targeting MEK1 and MEK2 for 72 h or 1 mM MS934 for 24 h. FIG. 17C shows immunoprecipitation (IP) of endogenous CRAF from HCT-116 cells treated with non-targeting siRNAs or siRNAs targeting a shared sequence amongst MEK1/2 (siMEK1/2 #1) for 24 or 48 h. The CRAF IPs were blotted with the indicated antibodies. Input samples were used as controls. The Cell Signaling Technology (CST) CRAF p-S621 was used. FIG. 17D shows the immunoblot analysis of CRAF phosphorylation in A549, NCI-H23, or PANC-1 cells treated with non-targeting siRNAs or siRNAs targeting a shared sequence amongst MEK1/2 (siMEK1/2 #1 or siMEK1/2 #2) for 72 h. FIGS. 17E-F show immunoblotting of CRAF phosphorylation at S621 levels in A549 cells treated with 1 mM MS934 alone or in combination with 2.5 nM Calyculin A for 0, 1, or 2 h. Cells pre-treated with Calyculin A for 30 minutes followed by MS934 treatment for indicated times. Blots are representative, and densitometric analysis of CRAF p-S621 (FIG. 17F) are means 1 SEM of 2 independent blots, each normalized to total CRAF protein and the loading control, GAPDH. FIG. 17G shows immunoprecipitation (IP) of endogenous CRAF from HCT-116 cells treated with 1 mM PD0325901 for 24 h. The CRAF IPs were blotted with the indicated antibodies. Input samples were used as controls. The GAPDH, CRAF, and MEK1/2 input samples are also shown in FIG. 10H, as they were analyzed at the same time. FIG. 17H shows the immunoblot analysis of CRAF total and p-S621, as well as MEK1/2 total and phosphorylated levels in HCT-116 cells treated with 0, 0.1 and 1 mM MS934 alone or combined with 1 mM RAF709 for 24 hours.

FIGS. 18A-G show MEK1/2-binding to CRAF and not MEK1/2 kinase activity is important for promoting CRAF phosphorylation at S621 and protein stability. FIG. 18A shows the immunoblot analysis of RAF phosphorylation in PANC-1 or PANC-1 cells stably expressing MEK1-GFP. Flag-ARAF, Flag-BRAF, or Flag-CRAF (all 2.5 μg) were transfected into PANC-1 cells. FIG. 18B shows immunoprecipitation (IP) of Flag-tagged CRAF constructs from PANC-1 cells stably expressing MEK1-GFP. The Flag-CRAF IPs were blotted with the indicated antibodies. IP of Flag-GFP and input samples were used as controls. FIG. 18C shows the immunoprecipitation (IP) of MYC-MEK1 constructs from PANC-1 cells. The MYC-MEK1 IPs were blotted with the indicated antibodies. Input samples were used as controls. FIG. 18D shows the immunoblot analysis of MEK1-GFP levels in PANC-1 cells expressing MEK1-GFP or MEK1-GFP(K97M) treated with 1 mM MS9343 for 24 hours. FIG. 18E shows the immunoprecipitation (IP) of MEK1-GFP constructs from PANC-1 cells. The GFP IPs were blotted with the indicated antibodies. Input samples were used as controls. FIGS. 18F-G shows the immunoblot analysis of endogenous CRAF total and p-S621 levels in HCT-116 cells expressing si-MEK1/2 #1 resistant MEK1-GFP(WT) or MEK1-GFP(IRI) treated with control, si-MEK1/2 #1 or MEK1/2 #2 siRNAs for 72 hours. Blots are representative, and densitometric analysis of p-S621 (I) are means 1 SEM from three blots, each normalized to the loading control, GAPDH.

FIGS. 19A-G show that MEK1/2 is important for the stabilization of mature but not immature newly synthesized CRAF which has anti-apoptosis functions. FIG. 19A shows immunoblotting for CRAF levels in A549 cells treated with DMSO, 1 mM MS934, 100 mg/ml cycloheximide, 0.05 mM STA9090, the combination of MS934 and cycloheximide or STA9090 for 24 h. FIG. 19B shows the immunoblot analysis of CRAF levels in HEK293T cells with DMSO, 1 mM MS934, 100 mg/ml cycloheximide, or the combination of MS934 and cycloheximide for 24 h. FIG. 19C shows the immunoblot analysis for p-S621 and CRAF levels in A549 cells treated with DMSO or escalating doses of MS934 in the presence or absence of 1 mM Torin-1 for 24 hours. FIGS. 19D-E show the immunoblot analysis of Flag-CRAF(WT) or Flag-CRAF(K375M) (FIG. 19D) or Flag-CRAF(S621A) (FIG. 19E) in PANC-1 cells stably expressing MEK1-GFP treated with 0, 0.1 or 1 mM MS934 for 24 h. FIGS. 19F-G show the immunoblot analysis for MYC-CRAF or MEK1/2 in PANC-1 cells stably expressing MEK1-GFP transfected with MYC-CRAF(WT) or MYC-CRAF(S471A) treated with 3.3 μM MS934 for 24 h. Blots are representative, and densitometric analysis of MYC-CRAF levels (FIG. 19G) are means±SEM from three blots, each normalized to the loading control, GAPDH.

FIGS. 20A-C show that in the absence of MEK1/2 immature CRAF has anti-apoptosis functions. FIG. 20A shows the immunoblot analysis of cleaved PARP levels in A549 cells following short hairpin RNA (shRNA)-mediated depletion of CRAF (shCRAF) or non-targeting shRNA(shNT) (1.0 μg/ml doxycycline for 96 h. FIGS. 20B-C show the bliss synergy score based on cell viability by CellTiter-Glo assay of HCT-116 cells treated with escalating doses of Navitoclax, PD0325901 (FIG. 20B), MS934 (FIG. 20C) or the combination for 5 days. Data were analyzed based on % of DMSO control, as means+SEM of three independent assays.

FIGS. 21A-C show that MEK1/2 bind to CRAF prior to RAS engagement forming a primed signalosome. FIG. 21A shows the immunoprecipitation (IP) of Flag-tagged CRAF constructs from PANC-1 cells stably expressing MEK1-GFP. The Flag-CRAF IPs were blotted with the indicated antibodies. IP of Flag-GFP and input samples were used as controls. FIG. 21B shows the immunoprecipitation (IP) of Flag-tagged CRAF constructs from HEK293T cells stably expressing MEK1-GFP. The Flag-CRAF IPs were blotted with the indicated antibodies. IP of Flag-GFP and input samples were used as controls. FIG. 21C shows the immunoblot analysis of pERK1/2 levels in PANC-1 (MEK1-GFP) cells transfected with Flag-tagged CRAF constructs.

FIG. 22 shows that MEK1/2 degradation or knockdown promotes CRAF but not ARAF or BRAF protein loss; depletion of MEK1/2 causes rapid CRAF dephosphorylation promoting its degradation; MEK1/2-binding to cRAF but not MEK1/2 catalytic activity promotes CRAF maturation; and a primed CRAF-MEK1/2 complex froms before activation by RAS engagement.

FIG. 23 shows a model for drug synergy combining MEK1/2 degraders with BCL2 inhibitors. In MEK1/2 competent cells, a ASK1/JNK signaling pathway negatively regulates BCL2 function promoting apoptosis. Under conditions of MEK1/2 degradation, accumulation of immature CRAF occurs leading to inhibition of ASK1/JNK activity and increased dependency on anti-apoptotic functions of BCL2. MS934-treated cells acquire enhanced sensitivity to BCL2 inhibition using inhibitors such as Navitoclax, where co-treatment of cells with MS934+Navitoclax causes increased apoptosis and blockade of cell viability.

### DETAILED DESCRIPTION

Unless defined otherwise, all technical and scientific terms have the same meaning as is commonly understood by one of ordinary skill in the art to which the embodiments disclosed belongs.

As used herein, the terms “a” or “an” means that “at least one” of “one or more” unless the context clearly indicates otherwise.

As used herein, the term “about” means that the numerical value is approximate and small variations would not significantly affect the practice of the disclosed embodiments. Where a numerical value can vary ±10% and remain with the scope of the disclosed embodiments.

As used herein, the terms “comprising” (and any form of comprising, such as “comprise”, “comprises” and “comprised”), “having” (and any form of having, such as “have” and “has”), “including” (and any form of including, such as “contains” and “contain”), are inclusive or open-ended and do not exclude additional, unrecited elements of method steps.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. For example, a subject who receives treatment with a dual RAF/MEK degrader, as described herein, in order to treat a cancer is “in need thereof” (i.e., as opposed to receiving a RAF inhibitor or an MEK inhibitor to treat a cancer).

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal of a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the terms “treat,” “treated,” or “treating” mean therapeutic treatment wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

It has been discovered (as described herein) that Proteolysis-Targeting Chimeras (PROTACs), such as MS934 (that utilize the MEK inhibitor PD0325901 and the VHL E3 ligase ligand) and MS432, degrade MEK1, MEK2, as well as the protein CRAF. These observations led to the examination, as described herein, at the mechanistic level where it was discovered that the reduction of CRAF protein by MS934 required MEK proteins, whereby knockdown of MEK1/2 rescued CRAF protein loss, suggesting that the feedback induced interaction of CRAF and MEK was required for CRAF degradation. CRAF has been shown to have kinase-independent growth and survival functions outside the MEK-ERK pathway in KRAS mutant cells, making CRAF degradation an attractive therapeutic avenue. Together, these findings support a method for degrading RAF protein in cells using MEK-degraders. MS934 is described in, for example, U.S. Patent Application Publication 2021/0395244.

The mitogen-activated protein kinase (MAPK) cascade is an evolutionarily conserved signaling pathway comprised of RAS GTPases, and downstream effector kinases (RAF, MEK, and ERK) that promote cell growth and survival (Bahar, M. E., et al. 2023. Signal Transduct Target Ther, 8, 455). The MAPK pathway is activated by growth factor receptor signaling inducing RAS GTPases to trigger activation of the RAF-MEK-ERK phosphorylation cascade, resulting in ERK-mediated regulation of various transcription factors that promote cell growth (Lavoie, H., et al. 2020. Nat Rev Mol Cell Biol, 21, 607-632; and Cargnello, M. & Roux, P. P. 2012. Microbiology and Molecular Biology Reviews, 76, 496-496). Frequent alteration of the MAPK signaling cascade occurs in human disease, with ~40% of cancers exhibiting perturbed MAPK signaling (Santarpia et al., 2012. Expert Opin Ther Targets, 16, 103-19). Moreover, activating mutations in important components of the MAPK cascade, such as KRAS or RAF kinases, are the underlying cause of several cancers. Consequently, the MAPK signaling pathway has been the focus of immense research leading to the development of inhibitors targeting the MAPK pathway. In particular, the MEK kinase has been a therapeutic target due to its central location within the MAPK pathway and highly druggable nature, with several MEK inhibitors currently being used to treat various MAPK-altered cancers.

MEK1 and MEK2 (MEK1/2) are ubiquitous members of the dual-specificity MAP2K kinase family that phosphorylate and activate ERK1/2 promoting cell growth and survival. MEK1/2 paralogs share highly similar kinase domains, including their activation loops, which are phosphorylated by the upstream RAF family of kinases ARAF, BRAF, and CRAF (Roskoski, 2012. Biochem Biophys Res Commun, 417, 5-10). MEK1/2 differ in their N-termini where MEK1 has a characterized ERK interaction. MEK1 contains an ERK negative feedback phosphorylation site within its proline-rich domain at T292 (Roskoski, 2012. Biochem Biophys Res Commun, 417, 5-10). Moreover, MEK1 but not MEK2 knockout is embryonic lethal, and MEK2 fails to compensate for MEK1 loss under specific settings (Bromberg-White et al., 2012. Brief Funct Genomics, 11, 300-10), hinting at differential cellular roles of MEK1/2. Notably, although the canonical function of MEK1/2 in activating downstream kinase ERK1/2 is well documented, little is known about whether MEK1/2 exhibits other functions in cells.

The activation of MEK1/2 by RAF is thoroughly described; RAF directly interacts with and phosphorylates MEK1/2, resulting in MEK1/2 kinase activation and subsequent phosphorylation of ERK1/2. In contrast, how the upstream RAF kinase, particularly CRAF, is activated is highly complex, involving an orchestration of chaperones, phosphorylation, and RAF dimerization, ultimately resulting in the phosphorylation of MEK1/2. The recognized CRAF signaling cascade has MEK1/2 functioning solely as a substrate of CRAF, binding to CRAF after RAS binding and RAF dimerization. However, there is evidence that MEK1 can directly regulate CRAF activity independent of RAS (Leicht et al., 2013. Biochimica et Biophysica Acta (BBA)—Molecular Cell Research, 1833, 976-986), suggesting that MEK1 has additional functions in the CRAF cycle. Here, using MEK1/2 proteolysis targeting chimeras (PROTACs), it is showns that MEK1/2 have kinase-independent functions in the regulation of CRAF protein stabilization, maturation, and kinase activation. These findings reshape the current view of the CRAF activation cycle, establishing MEK1/2 binding to CRAF as an early required stabilizing event preceding CRAF-RAS engagement. Thus, MEK1/2 functions as both an activator and substrate of CRAF.

Altered MAPK signaling frequently occurs in human disease. MEK1 and MEK2 (MEK1/2) are central protein kinases in the MAPK signaling cascade that phosphorylate ERK1/2 promoting cell growth. MEK1/2 degraders offer a strategy to characterize both kinase-dependent and independent functions of MEK1/2. Described herein are findings demonstrating that MEK1/2 degradation, but not kinase inhibition, caused the subsequent degradation of upstream kinase CRAF via a cell-intrinsic mechanism. MEK1/2 binding to CRAF, but not MEK1/2 catalytic activity, was required for CRAF protein stability and maturation to a functional kinase. In the absence of MEK1/2, a minor pool of newly synthesized immature CRAF that had anti-apoptotic functions remained. Finally, the results show that a stable primed CRAF-MEK1/2 signaling complex existed in cells that required RAS binding to potentiate MEK-ERK phosphorylation. Together, the results described herein demonstrate a previously unrecognized kinase-independent function of MEK1/2, while contextualizing MEK1/2 as an integral component of the CRAF activation cycle beyond the conventional CRAF-MEK kinase-substrate paradigm.

The present disclosure provides methods of treating a human disease that requires RAF-MEK in a subject in need thereof, the method comprising administering a dual RAF/MEK degrader to the subject.

The present disclosure also provides methods of treating a cancer in a subject in need thereof, the methods comprising administering a therapeutically effective amount of a MEK1/2 degrader and a therapeutically effective amount of a BCL-2 inhibitor to the subject.

In some of the embodiments, the human disease utilizes the RAF-MEK-ERK pathway. In some embodiments, the human disease is a cancer having an alteration in a receptor-tyrosine kinase. In some embodiments, the human disease is a RAS-altered cancer. As used herein, a “RAS-altered cancer” refers to a subject having an alteration in a RAS gene family member. In some embodiments, the RAS-altered cancer is a KRAS-altered cancer. As used herein, a “KRAS-altered cancer” refers to a subject having an alteration in the KRAS gene. In some embodiments, the RAS-altered cancer is an NRAS-altered cancer. As used herein, a “NRAS-altered cancer” refers to a subject having an alteration in the NRAS gene. In some embodiments, the RAS-altered cancer is an HRAS-altered cancer. As used herein, a “HRAS-altered cancer” refers to a subject having an alteration in the HRAS gene. In some embodiments, the RAS-altered cancer includes cancers with alterations in BRAF, NF1, MEK, ERK, MYC, EGFR, HER2, and/or FGFR. In some of the embodiments, the human disease is a RAF-altered cancer. As used herein, a “RAF-altered cancer” refers to a subject having an alteration in a RAF gene family member. In some of the embodiments, the RAF-altered cancer is a BRAF mutant cancer. In some of the embodiments, the BRAF mutant cancer is BRAF(V600E) cancer. In some of the embodiments, the BRAF(V600E) cancer is melanoma or colorectal cancer. In some of the embodiments, the RAF-altered cancer is a CRAF-altered cancer. As used herein, a “CRAF-altered cancer” refers to a subject having an alteration in the CRAF gene. In some of the embodiments, the human disease is an MEK-altered cancer. As used herein, a “MEK-altered cancer” refers to a subject having an alteration in a MEK gene family member. In some of the embodiments, the human disease is an ERK-altered cancer. As used herein, a “ERK-altered cancer” refers to a subject having an alteration in a ERK gene family member.

As used herein, the terms “altered” or “alteration” can refer to an altered gene that has undergone a genetic mutation or alternation causing it to produce an abnormal protein that can drive cancer development or promotes cancer or increases the susceptibility of cancer developing. In some aspects, these alterations can be somatic (not inherited) and can develop over time. In some aspects, the alterations can be single-base substitutions. In some aspects, the alteration can be a molecular alteration. Examples of molecular alterations include but are not limited to one or more cancer-driving mutations, gene fusions, amplification, deletion, and post-translations modifications. For example, a CRAF-altered cancer can include one or more somatic mutations or overexpression of the RAF1 gene.

In some of the embodiments, the human disease is an RASopathy. In some of the embodiments, the RASopathy is Noonan syndrome. In some of the embodiments, the human disease is NF1 syndrome. In some of the embodiments, the human disease is NF2 syndrome. In some of the embodiments, the human disease is syndrome neurofibromatosis Type 1, Type 2, or Type 3 (Schwannomatosis). In some of the embodiments, the human disease is CoQ-deficiency kidney disease.

In some embodiments, the cancer is lung cancer (e.g., non-small-cell lung cancer, small-cell lung cancer), breast cancer, prostate cancer, ovarian cancer, testicular cancer, colon cancer, colorectal cancer, renal cancer (e.g., renal cell carcinoma), bladder cancer, pancreatic cancer, glioblastoma, neuroblastoma, retinoblastoma, leukemia (e.g., acute myeloid leukemia), melanoma, kidney cancer, bone cancer (e.g., osteosarcoma), appendiceal cancer, small bowel cancer, ampullary cancer, cervical cancer, endometrial cancer, gastrointestinal neuroendocrine tumor, uterine endometrioid carcinoma, germ cell tumor, oesophagogastric cancer, sex cord stromal tumor, hepatobiliary cancer, histiocytosis, anal cancer, thyroid cancer, mature B cell neoplasm, soft-tissue sarcoma, gastrointestinal stromal tumor, skin cancer (non-melanoma), head and neck cancer, glioma, salivary gland cancer, mature B cell lymphoma, or a thymic tumor. In some embodiments, the cancer is lung cancer. In some embodiments, the cancer is breast cancer. In some embodiments, the cancer is prostate cancer. In some embodiments, the cancer is ovarian cancer. In some embodiments, the cancer is testicular cancer. In some embodiments, the cancer is colon cancer. In some embodiments, the cancer is colorectal cancer. In some embodiments, the cancer is renal cancer. In some embodiments, the cancer is bladder cancer. In some embodiments, the cancer is pancreatic cancer. In some embodiments, the cancer is glioblastoma. In some embodiments, the cancer is neuroblastoma. In some embodiments, the cancer is retinoblastoma. In some embodiments, the cancer is leukemia. In some embodiments, the cancer is melanoma. In some embodiments, the cancer is kidney cancer. In some embodiments, the cancer is osteosarcoma. In some embodiments, the cancer is colon cancer, non-small lung cancer, or pancreatic cancer. In some embodiments, the cancer is non-small lung cancer. In some embodiments, the cancer is appendiceal cancer. In some embodiments, the cancer is small bowel cancer. In some embodiments, the cancer is ampullary cancer. In some embodiments, the cancer is cervical cancer. In some embodiments, the cancer is endometrial cancer. In some embodiments, the cancer is gastrointestinal neuroendocrine tumor. In some embodiments, the cancer is uterine endometrioid carcinoma. In some embodiments, the cancer is germ cell tumor. In some embodiments, the cancer is oesophagogastric cancer. In some embodiments, the cancer is sex cord stromal tumor. In some embodiments, the cancer is hepatobiliary cancer. In some embodiments, the cancer is histiocytosis. In some embodiments, the cancer is anal cancer. In some embodiments, the cancer is thyroid cancer. In some embodiments, the cancer is mature B cell neoplasm. In some embodiments, the cancer is soft-tissue sarcoma. In some embodiments, the cancer is gastrointestinal stromal tumor. In some embodiments, the cancer is skin cancer (non-melanoma). In some embodiments, the cancer is head and neck cancer. In some embodiments, the cancer is glioma. In some embodiments, the cancer is salivary gland cancer. In some embodiments, the cancer is mature B cell lymphoma. In some embodiments, the cancer is a thymic tumor. In any of the embodiments described herein, the cancer can be one that requires inhibition or degradation of both CRAF and MEK1 and/or MEK2.

In some embodiments, the RAS-altered cancer is non-small lung cancer, breast cancer, prostate cancer, ovarian cancer, testicular cancer, colon cancer, colorectal cancer, renal cancer, bladder cancer, pancreatic cancer, glioblastoma, neuroblastoma, retinoblastoma, leukemia, melanoma, kidney cancer, or osteosarcoma. In some embodiments, the RAS-altered cancer is colon cancer, colorectal cancer, non-small lung cancer, or pancreatic cancer.

In some embodiments, the dual RAF/MEK degrader is an ARAF/MEK degrader. In some embodiments, the dual RAF/MEK degrader is a BRAF/MEK degrader. In some embodiments, the dual RAF/MEK degrader is a CRAF/MEK degrader. In some embodiments, the dual CRAF/MEK degrader comprises MS934, MS432, MS928, MS910, or any combination thereof. In some embodiments, the dual CRAF/MEK degrader is MS934 (which utilizes the MEK inhibitor PD0325901 and the VHL E3 ligase ligand). In some embodiments, the dual CRAF/MEK degrader is MS432. In some embodiments, the dual CRAF/MEK degrader is MS928. In some embodiments, the dual CRAF/MEK degrader is MS910. In some embodiments, the dual RAF/MEK degrader is any PROTAC that incorporates an MEK1/2 inhibitor that causes collateral degradation of RAF.

In any of the embodiments herein, the subject can also be administered an MEK inhibitor, a MEK1/2 degrader, a BRAF inhibitor, a PI3K inhibitor, an MTOR inhibitor, an SHP2 inhibitor, a KRAS inhibitor, an AKT inhibitor, an ERK inhibitor, an ERBB inhibitor, an ABCB1(MDR1) inhibitor, a CXCR1/2 inhibitor, a PD-L1/PD-1 inhibitor, an RTK inhibitor, an SOS1 inhibitor, a CDK4/6 inhibitor, an AURKA inhibitor, a WEE1 inhibitor, or an mTORC1 inhibitor, a BET bromodomain protein inhibitor, an HDAC inhibitor, a BCL-2 inhibitor, or any combination thereof. In any of the embodiments herein, the subject can also be administered an siRNA or a cancer vaccine. In any of the embodiments, described herein, the subject can also be administered an anti-cancer agent, such as a chemotherapeutic agent. In any of the embodiments, described herein, the subject can also be administered an immunotherapeutic agent. In any of the embodiments herein, the subject can also undergo radiation therapy.

In some embodiments, the subject can also be administered an MEK inhibitor. In some embodiments, the MEK inhibitor comprises trametinib (e.g., GSK1120212), cobimetinib (e.g., GDC-0973), binimetinib (e.g., MEK162), mirdametinib, TAK733, selumetinib (e.g., AZD6244), refametinib (e.g., BAY869766), pimasertib (e.g., AS703026), PD98059, U0126, Ro 09-2210, CI-1040, PD0325901, R04987655, R05126766, GDC-0623, G-573, E6201, AZD8330, or WX-554, or any combination thereof. In some embodiments, the MEK inhibitor comprises trametinib, cobimetinib, binimetinib, mirdametinib, TAK733, selumetinib, refametinib, pimasertib, PD98059, U0126, Ro 09-2210, CI-1040, PD0325901, R04987655, R05126766, GDC-0623, G-573, E6201, AZD8330, or WX-554, or any combination thereof. In some embodiments, the MEK inhibitor comprises trametinib. In some embodiments, the MEK inhibitor comprises cobimetinib. In some embodiments, the MEK inhibitor comprises binimetinib. In some embodiments, the MEK inhibitor comprises mirdametinib. In some embodiments, the MEK inhibitor comprises refametinib. In some embodiments, the MEK inhibitor comprises pimasertib. In some embodiments, the MEK inhibitor comprises PD98059. In some embodiments, the MEK inhibitor comprises U0126. In some embodiments, the MEK inhibitor comprises Ro 09-2210. In some embodiments, the MEK inhibitor comprises CI-1040. In some embodiments, the MEK inhibitor comprises PD0325901. In some embodiments, the MEK inhibitor comprises R04987655. In some embodiments, the MEK inhibitor comprises R05126766. In some embodiments, the MEK inhibitor comprises GDC-0623. In some embodiments, the MEK inhibitor comprises G-573. In some embodiments, the MEK inhibitor comprises E6201. In some embodiments, the MEK inhibitor comprises AZD8330. In some embodiments, the MEK inhibitor comprises WX-554. In some embodiments, the MEK inhibitor comprises TAK733. In some embodiments, the MEK inhibitor comprises selumetinib.

In some aspects, the MEK1/2 degrader can be MS934.

In some aspects, the subject can be administered a MEK1/2 degrader and a BCL-2 inhibitor.

In some aspects, the BCL-2 inhibitor can be naritoclax, venetoclax, obatoclax, or oblimersen sodium,

In some embodiments, the subject can also be administered a BRAF inhibitor. In some embodiments, the BRAF inhibitor comprises vemurafenib (e.g., PLX4032), sorafenib (e.g., BAY 43-9006), dabrafenib (e.g., GSK2118436), encorafenib (e.g., LGX818), GDC-0879, or PLX-4720, Raf inhibitor 2, GNE-9815, TBAP-001, Raf inhibitor 1, regorafenib (e.g., BAY 73-4506), doramapimod (e.g., BIRB 796), RAF265 (e.g., CHIR-265), NVP-BHG712, SB590885, ZM 336372, AZ 628, GW5074, regorafenib (e.g., BAY-734506), B-Raf IN 1, B-Raf inhibitor 1 (Compound 13) dihydrochloride, L-779450, MCP110, tovorafenib (e.g., MLN2480), RO5126766 (e.g., CH5126766), TAK-632, CCT196969, LY3009120, lifirafenib (e.g., BGB-283), PLX7904, PLX8394, agerafenib (e.g., RXDX-105), BAW2881 (e.g., NVP-BAW2881), RAF709, naporafenib (e.g., LXH254), AZ304, or belvarafenib (e.g., HM95573), or any combination thereof. In some embodiments, the BRAF inhibitor comprises vemurafenib. In some embodiments, the BRAF inhibitor comprises sorafenib. In some embodiments, the BRAF inhibitor comprises dabrafenib. In some embodiments, the BRAF inhibitor comprises encorafenib. In some embodiments, the BRAF inhibitor comprises GDC-0879. In some embodiments, the BRAF inhibitor comprises PLX-4720. In some embodiments, the BRAF inhibitor comprises Raf inhibitor 2. In some embodiments, the BRAF inhibitor comprises GNE-9815. In some embodiments, the BRAF inhibitor comprises TBAP-001. In some embodiments, the BRAF inhibitor comprises Raf inhibitor 1. In some embodiments, the BRAF inhibitor comprises regorafenib. In some embodiments, the BRAF inhibitor comprises doramapimod. In some embodiments, the BRAF inhibitor comprises RAF265. In some embodiments, the BRAF inhibitor comprises NVP-BHG712. In some embodiments, the BRAF inhibitor comprises SB590885. In some embodiments, the BRAF inhibitor comprises ZM 336372. In some embodiments, the BRAF inhibitor comprises AZ 628. In some embodiments, the BRAF inhibitor comprises GW5074. In some embodiments, the BRAF inhibitor comprises regorafenib. In some embodiments, the BRAF inhibitor comprises B-Raf IN 1. In some embodiments, the BRAF inhibitor comprises B-Raf inhibitor 1 (Compound 13) dihydrochloride. In some embodiments, the BRAF inhibitor comprises L-779450. In some embodiments, the BRAF inhibitor comprises MCP110. In some embodiments, the BRAF inhibitor comprises tovorafenib. In some embodiments, the BRAF inhibitor comprises RO5126766. In some embodiments, the BRAF inhibitor comprises TAK-632. In some embodiments, the BRAF inhibitor comprises CCT196969. In some embodiments, the BRAF inhibitor comprises LY3009120. In some embodiments, the BRAF inhibitor comprises lifirafenib. In some embodiments, the BRAF inhibitor comprises PLX7904. In some embodiments, the BRAF inhibitor comprises PLX8394. In some embodiments, the BRAF inhibitor comprises agerafenib. In some embodiments, the BRAF inhibitor comprises BAW2881. In some embodiments, the BRAF inhibitor comprises RAF709. In some embodiments, the BRAF inhibitor comprises naporafenib. In some embodiments, the BRAF inhibitor comprises AZ304. In some embodiments, the BRAF inhibitor comprises belvarafenib.

In some embodiments, the subject can also be administered a PI3K inhibitor. In some embodiments, the PI3K inhibitor comprises ZSTK474, idelalisib, copanlisib (e.g., BAY 80-6946), duvelisib (e.g., IPI-145), alpelisib (e.g., BYL719), umbralisib (e.g., TGR-1202), leniolisib (e.g., CDZ 173), parsaclisib (e.g., INCB050465), zandelisib, eganelisib (e.g., IPI-549), linperlisib, nemiralisib, pilaralisib (e.g., XL147), seletalisib (e.g., UCB-5857), tenalisib (e.g., RP6530), AMG 319, AZD8186, AZD8835, CAL263, TG100-115, IC87114, dactolisib (e.g., BEZ235), pictilisib (e.g., GDC-0941), LY294002, buparlisib (e.g., BKM120), PI-103, NU7441 (e.g., KU-57788), TGX-221, IC-87114, Wortmannin (e.g., KY 12420), XL147 analogue, AS-605240, PIK-75 HCl, Rigosertib (e.g., ON-01910), 3-Methyladenine (3-MA), A66, voxtalisib (XL765) analogue, omipalisib (e.g., GSK2126458), PIK-90, AZD6482, PF-04691502, apitolisib (e.g., GDC-0980), GSK1059615, gedatolisib (e.g., PKI-587), AS-252424, NU7026, BGT226 (e.g., NVP-BGT226) maleate, fimepinostat (e.g., CUDC-907), PIK-294, AS-604850, GSK2636771, YM201636, CH5132799, CAY10505, PIK-293, PKI-402, PI3K/Akt inhibitor library, TG100713, VS-5584 (e.g., SB2343), taselisib (e.g., GDC 0032), KU-0060648, CZC24832, demethyl-Coclaurine Oroxin B, Homosalate, cinobufagin, resibufogenin, hispidulin, GSK2292767, lanatoside C, PI3K/mTOR Inhibitor-2, zeaxanthin, disitertide (e.g., P144), cafestol, paxalisib (e.g., GDC-0084), SKI-V, Dichroa febrifuga extract, MTX-211, trichosanthis pericarpium extract, Dioscoreae nipponicae Rhizoma extract, 740 Y-P (e.g., PDGFR 740Y-P), GDC-0326, PIK-108, HS-173, SF2523, lupenone, serabelisib (e.g., TAK-117), quercetin (e.g., NSC 9221), bimiralisib (e.g., PQR309), VPS34 inhibitor 1 (Compound 19), IHMT-PI3KM-372, voxtalisib (e.g., XL765), autophinib, GNE-317, (E)-Akt inhibitor-IV, notoginsenoside R1, solasodine, galleon, pectolinarin, inavolisib (e.g., GDC-0077), SRX3207, α-linolenic acid, acalisib (e.g., GS-9820), ME-401, samotolisib (e.g., LY3023414), VPS34-IN1, ailanthone, Tripterygium wilfordii extract, IPI-3063, SAR405, PIK-III, PI-3065, quercetin dihydrate, SPP-86, erucic acid, trigonelline, deguelin, selective PI3Kδ Inhibitor 1 (compound 7n), amarogentin, Poria Cocos extract, loureirin A, YS-49, PF-4989216, GNE-477, or GNE-493, or any combination thereof. In some embodiments, the PI3K inhibitor comprises ZSTK474. In some embodiments, the PI3K inhibitor comprises idelalisib. In some embodiments, the PI3K inhibitor comprises copanlisib. In some embodiments, the PI3K inhibitor comprises duvelisib. In some embodiments, the PI3K inhibitor comprises alpelisib. In some embodiments, the PI3K inhibitor comprises umbralisib. In some embodiments, the PI3K inhibitor comprises leniolisib. In some embodiments, the PI3K inhibitor comprises parsaclisib. In some embodiments, the PI3K inhibitor comprises zandelisib. In some embodiments, the PI3K inhibitor comprises eganelisib. In some embodiments, the PI3K inhibitor comprises linperlisib. In some embodiments, the PI3K inhibitor comprises nemiralisib. In some embodiments, the PI3K inhibitor comprises pilaralisib. In some embodiments, the PI3K inhibitor comprises seletalisib. In some embodiments, the PI3K inhibitor comprises tenalisib. In some embodiments, the PI3K inhibitor comprises AMG 319. In some embodiments, the PI3K inhibitor comprises AZD8186. In some embodiments, the PI3K inhibitor comprises AZD8835. In some embodiments, the PI3K inhibitor comprises CAL263. In some embodiments, the PI3K inhibitor comprises TG100-115. In some embodiments, the PI3K inhibitor comprises IC87114. In some embodiments, the PI3K inhibitor comprises dactolisib. In some embodiments, the PI3K inhibitor comprises pictilisib. In some embodiments, the PI3K inhibitor comprises LY294002. In some embodiments, the PI3K inhibitor comprises buparlisib. In some embodiments, the PI3K inhibitor comprises PI-103. In some embodiments, the PI3K inhibitor comprises NU7441. In some embodiments, the PI3K inhibitor comprises TGX-221. In some embodiments, the PI3K inhibitor comprises IC-87114. In some embodiments, the PI3K inhibitor comprises Wortmannin. In some embodiments, the PI3K inhibitor comprises XL147 analogue. In some embodiments, the PI3K inhibitor comprises AS-605240. In some embodiments, the PI3K inhibitor comprises PIK-75 HCl. In some embodiments, the PI3K inhibitor comprises rigosertib. In some embodiments, the PI3K inhibitor comprises 3-Methyladenine (3-MA). In some embodiments, the PI3K inhibitor comprises A66. In some embodiments, the PI3K inhibitor comprises voxtalisib (XL765) analogue. In some embodiments, the PI3K inhibitor comprises omipalisib. In some embodiments, the PI3K inhibitor comprises PIK-90. In some embodiments, the PI3K inhibitor comprises AZD6482. In some embodiments, the PI3K inhibitor comprises PF-04691502. In some embodiments, the PI3K inhibitor comprises apitolisib. In some embodiments, the PI3K inhibitor comprises GSK1059615. In some embodiments, the PI3K inhibitor comprises gedatolisib. In some embodiments, the PI3K inhibitor comprises AS-252424. In some embodiments, the PI3K inhibitor comprises NU7026. In some embodiments, the PI3K inhibitor comprises BGT226 maleate. In some embodiments, the PI3K inhibitor comprises fimepinostat. In some embodiments, the PI3K inhibitor comprises PIK-294. In some embodiments, the PI3K inhibitor comprises AS-604850. In some embodiments, the PI3K inhibitor comprises GSK2636771. In some embodiments, the PI3K inhibitor comprises YM201636. In some embodiments, the PI3K inhibitor comprises CH5132799. In some embodiments, the PI3K inhibitor comprises CAY10505. In some embodiments, the PI3K inhibitor comprises PIK-293. In some embodiments, the PI3K inhibitor comprises PKI-402. In some embodiments, the PI3K inhibitor comprises PI3K/Akt inhibitor library. In some embodiments, the PI3K inhibitor comprises TG100713. In some embodiments, the PI3K inhibitor comprises VS-5584. In some embodiments, the PI3K inhibitor comprises taselisib. In some embodiments, the PI3K inhibitor comprises KU-0060648. In some embodiments, the PI3K inhibitor comprises CZC24832. In some embodiments, the PI3K inhibitor comprises demethyl-Coclaurine Oroxin B. In some embodiments, the PI3K inhibitor comprises homosalate. In some embodiments, the PI3K inhibitor comprises cinobufagin. In some embodiments, the PI3K inhibitor comprises resibufogenin. In some embodiments, the PI3K inhibitor comprises hispidulin. In some embodiments, the PI3K inhibitor comprises GSK2292767. In some embodiments, the PI3K inhibitor comprises lanatoside C. In some embodiments, the PI3K inhibitor comprises PI3K/mTOR Inhibitor-2. In some embodiments, the PI3K inhibitor comprises zeaxanthin. In some embodiments, the PI3K inhibitor comprises disitertide. In some embodiments, the PI3K inhibitor comprises cafestol. In some embodiments, the PI3K inhibitor comprises paxalisib. In some embodiments, the PI3K inhibitor comprises SKI-V. In some embodiments, the PI3K inhibitor comprises Dichroa febrifuga extract. In some embodiments, the PI3K inhibitor comprises MTX-211. In some embodiments, the PI3K inhibitor comprises trichosanthis pericarpium extract. In some embodiments, the PI3K inhibitor comprises Dioscoreae nipponicae Rhizoma extract. In some embodiments, the PI3K inhibitor comprises 740 Y-P. In some embodiments, the PI3K inhibitor comprises GDC-0326. In some embodiments, the PI3K inhibitor comprises PIK-108. In some embodiments, the PI3K inhibitor comprises HS-173. In some embodiments, the PI3K inhibitor comprises SF2523. In some embodiments, the PI3K inhibitor comprises lupenone. In some embodiments, the PI3K inhibitor comprises serabelisib. In some embodiments, the PI3K inhibitor comprises quercetin. In some embodiments, the PI3K inhibitor comprises bimiralisib. In some embodiments, the PI3K inhibitor comprises VPS34 inhibitor 1 (Compound 19). In some embodiments, the PI3K inhibitor comprises IHMT-PI3Kδ-372. In some embodiments, the PI3K inhibitor comprises voxtalisib. In some embodiments, the PI3K inhibitor comprises autophinib. In some embodiments, the PI3K inhibitor comprises GNE-317. In some embodiments, the PI3K inhibitor comprises (E)-Akt inhibitor-IV. In some embodiments, the PI3K inhibitor comprises notoginsenoside R1. In some embodiments, the PI3K inhibitor comprises solasodine. In some embodiments, the PI3K inhibitor comprises galleon. In some embodiments, the PI3K inhibitor comprises pectolinarin. In some embodiments, the PI3K inhibitor comprises inavolisib. In some embodiments, the PI3K inhibitor comprises SRX3207. In some embodiments, the PI3K inhibitor comprises α-linolenic acid. In some embodiments, the PI3K inhibitor comprises acalisib. In some embodiments, the PI3K inhibitor comprises ME-401. In some embodiments, the PI3K inhibitor comprises samotolisib. In some embodiments, the PI3K inhibitor comprises VPS34-IN1. In some embodiments, the PI3K inhibitor comprises ailanthone. In some embodiments, the PI3K inhibitor comprises Tripterygium wilfordii extract. In some embodiments, the PI3K inhibitor comprises IPI-3063. In some embodiments, the PI3K inhibitor comprises SAR405. In some embodiments, the PI3K inhibitor comprises PIK-III. In some embodiments, the PI3K inhibitor comprises PI-3065. In some embodiments, the PI3K inhibitor comprises quercetin dihydrate. In some embodiments, the PI3K inhibitor comprises SPP-86. In some embodiments, the PI3K inhibitor comprises erucic acid. In some embodiments, the PI3K inhibitor comprises trigonelline. In some embodiments, the PI3K inhibitor comprises deguelin. In some embodiments, the PI3K inhibitor comprises selective PI3Kδ Inhibitor 1 (compound 7n). In some embodiments, the PI3K inhibitor comprises amarogentin. In some embodiments, the PI3K inhibitor comprises Poria Cocos extract. In some embodiments, the PI3K inhibitor comprises loureirin A. In some embodiments, the PI3K inhibitor comprises YS-49. In some embodiments, the PI3K inhibitor comprises PF-4989216. In some embodiments, the PI3K inhibitor comprises GNE-477. In some embodiments, the PI3K inhibitor comprises GNE-493.

In some embodiments, the subject can also be administered an MTOR inhibitor. In some embodiments, the MTOR inhibitor comprises PI3K/mTOR Inhibitor-2, mTOR inhibitor-1, dactolisib (e.g., BEZ235), rapamycin (e.g., AY-22989), everolimus (e.g., RAD001), AZD8055, temsirolimus (e.g., CCI-779), PI-103, NU7441 (e.g., KU-57788), KU-0063794, torkinib (e.g., PP242), ridaforolimus (e.g., deforolimus, MK-8669), sapanisertib (e.g., MLN0128), voxtalisib (XL765) analogue, torin 1, omipalisib (e.g., GSK2126458), OSI-027, PF-04691502, apitolisib (e.g., GDC-0980), GSK1059615, gedatolisib (e.g., PKI-587), WYE-354, vistusertib (e.g., AZD2014), torin 2, WYE-125132 (e.g., WYE-132), BGT226 (e.g., NVP-BGT226) maleate, palomid 529 (P529), PP121, WYE-687, nitazoxanide (e.g., NSC 697855), WAY-600, GDC-0349, XL388, 4EGI-1, JR-AB2-011, rotundic acid, lanatoside C, Compound 401, astragaloside IV, ginkgolide K, CC-115, paxalisib (e.g., GDC-0084), CZ415, SF2523, bimiralisib (e.g., PQR309), voxtalisib (e.g., XL765), chrysophanic acid, onatasertib (e.g., CC 223), MTI-31, ABTL-0812, MHY-1685, GNE-477, GNE-493, ETP-46464, zotarolimus (e.g., ABT-578), or PQR620, or any combination thereof. In some embodiments, the MTOR inhibitor comprises PI3K/mTOR Inhibitor-2. In some embodiments, the MTOR inhibitor comprises mTOR inhibitor-1. In some embodiments, the MTOR inhibitor comprises dactolisib. In some embodiments, the MTOR inhibitor comprises rapamycin. In some embodiments, the MTOR inhibitor comprises everolimus. In some embodiments, the MTOR inhibitor comprises AZD8055. In some embodiments, the MTOR inhibitor comprises temsirolimus. In some embodiments, the MTOR inhibitor comprises PI-103. In some embodiments, the MTOR inhibitor comprises NU7441. In some embodiments, the MTOR inhibitor comprises KU-0063794. In some embodiments, the MTOR inhibitor comprises torkinib. In some embodiments, the MTOR inhibitor comprises ridaforolimus. In some embodiments, the MTOR inhibitor comprises sapanisertib. In some embodiments, the MTOR inhibitor comprises voxtalisib (XL765) analogue. In some embodiments, the MTOR inhibitor comprises torin 1. In some embodiments, the MTOR inhibitor comprises omipalisib. In some embodiments, the MTOR inhibitor comprises OSI-027. In some embodiments, the MTOR inhibitor comprises PF-04691502. In some embodiments, the MTOR inhibitor comprises apitolisib. In some embodiments, the MTOR inhibitor comprises GSK1059615. In some embodiments, the MTOR inhibitor comprises gedatolisib. In some embodiments, the MTOR inhibitor comprises WYE-354. In some embodiments, the MTOR inhibitor comprises vistusertib. In some embodiments, the MTOR inhibitor comprises torin 2. In some embodiments, the MTOR inhibitor comprises WYE-125132. In some embodiments, the MTOR inhibitor comprises BGT226 maleate. In some embodiments, the MTOR inhibitor comprises palomid 529 (P529). In some embodiments, the MTOR inhibitor comprises PP121. In some embodiments, the MTOR inhibitor comprises WYE-687. In some embodiments, the MTOR inhibitor comprises nitazoxanide. In some embodiments, the MTOR inhibitor comprises WAY-600. In some embodiments, the MTOR inhibitor comprises GDC-0349. In some embodiments, the MTOR inhibitor comprises XL388. In some embodiments, the MTOR inhibitor comprises 4EGI-1. In some embodiments, the MTOR inhibitor comprises JR-AB2-011. In some embodiments, the MTOR inhibitor comprises rotundic acid. In some embodiments, the MTOR inhibitor comprises lanatoside C. In some embodiments, the MTOR inhibitor comprises Compound 401. In some embodiments, the MTOR inhibitor comprises stragaloside IV. In some embodiments, the MTOR inhibitor comprises ginkgolide K. In some embodiments, the MTOR inhibitor comprises CC-115. In some embodiments, the MTOR inhibitor comprises paxalisib. In some embodiments, the MTOR inhibitor comprises CZ415. In some embodiments, the MTOR inhibitor comprises SF2523. In some embodiments, the MTOR inhibitor comprises bimiralisib. In some embodiments, the MTOR inhibitor comprises voxtalisib. In some embodiments, the MTOR inhibitor comprises chrysophanic acid. In some embodiments, the MTOR inhibitor comprises onatasertib. In some embodiments, the MTOR inhibitor comprises MTI-31. In some embodiments, the MTOR inhibitor comprises ABTL-0812. In some embodiments, the MTOR inhibitor comprises MHY-1685. In some embodiments, the MTOR inhibitor comprises GNE-477. In some embodiments, the MTOR inhibitor comprises GNE-493. In some embodiments, the MTOR inhibitor comprises ETP-46464. In some embodiments, the MTOR inhibitor comprises zotarolimus. In some embodiments, the MTOR inhibitor comprises PQR620.

In some embodiments, the subject can also be administered an SHP2 inhibitor. In some embodiments, the SHP2 inhibitor comprises GDC-1971, TNO155, SHP099, sodium stibogluconate, IACS-13909, RMC-4550, SHP099 HCl, PHPS1, NSC87877, BVT-948, SPI-112, RMC-4630, JAB-3068, JAB-3312, or ERAS-601, or any combination thereof. In some embodiments, the SHP2 inhibitor comprises GDC-1971. In some embodiments, the SHP2 inhibitor comprises TNO155. In some embodiments, the SHP2 inhibitor comprises SHP099. In some embodiments, the SHP2 inhibitor comprises sodium stibogluconate. In some embodiments, the SHP2 inhibitor comprises IACS-13909. In some embodiments, the SHP2 inhibitor comprises RMC-4550. In some embodiments, the SHP2 inhibitor comprises SHP099 HCl. In some embodiments, the SHP2 inhibitor comprises PHPS1. In some embodiments, the SHP2 inhibitor comprises NSC87877. In some embodiments, the SHP2 inhibitor comprises BVT-948. In some embodiments, the SHP2 inhibitor comprises SPI-112. In some embodiments, the SHP2 inhibitor comprises RMC-4630. In some embodiments, the SHP2 inhibitor comprises JAB-3068. In some embodiments, the SHP2 inhibitor comprises JAB-3312. In some embodiments, the SHP2 inhibitor comprises ERAS-601.

In some embodiments, the subject can also be administered an KRAS inhibitor. In some embodiments, the KRAS inhibitor comprises zoledronic acid (e.g., ZOL 446), lonafarnib (e.g., SCH66336), K-Ras(G12C) inhibitor 9, salirasib, MRTX1133, alamandine, sotorasib (e.g., AMG510), LC-2, (Rac)-Antineoplaston, A10, K-Ras-IN-1, GDC6036, deltarasin, APS6-45, BAY-293, MCP110, 6H05, K-Ras(G12C) inhibitor 12, Kobe0065, K-Ras(G12C) inhibitor 6, BQU57, ARS-1620, JDQ443, Kobe2602, BI-2852, NAV-2729, antineoplaston A10, fendiline hydrochloride, adagrasib (e.g., MRTX849), KRpep-2d, ARS-853 (e.g., ARS853), BI-3406, perillyl alcohol, ASP2453, RBC8, sotorasib (e.g., AMG510) racemate, Pan-RAS-IN-1, MRTX-1257, KY1220, CID-1067700, zoledronic acid monohydrate, D-1553, RG6330/GDC-6036, BI-1823911, JAB-21822, LY3537982, JNJ-74699157, MK-1084, JAB-22000, JAB-23000, RM-018, RMC-6291, RMC-9805/RM-036, RMC-8839, RMC-6236, JAB-23400, or BBP-454, or any combination thereof. In some embodiments, the KRAS inhibitor comprises zoledronic acid. In some embodiments, the KRAS inhibitor comprises lonafarnib. In some embodiments, the KRAS inhibitor comprises K-Ras(G12C) inhibitor 9. In some embodiments, the KRAS inhibitor comprises salirasib. In some embodiments, the KRAS inhibitor comprises MRTX1133. In some embodiments, the KRAS inhibitor comprises alamandine. In some embodiments, the KRAS inhibitor comprises sotorasib. In some embodiments, the KRAS inhibitor comprises LC-2. In some embodiments, the KRAS inhibitor comprises (Rac)-Antineoplaston. In some embodiments, the KRAS inhibitor comprises A10. In some embodiments, the KRAS inhibitor comprises K-Ras-IN-1. In some embodiments, the KRAS inhibitor comprises GDC6036. In some embodiments, the KRAS inhibitor comprises deltarasin. In some embodiments, the KRAS inhibitor comprises APS6-45. In some embodiments, the KRAS inhibitor comprises BAY-293. In some embodiments, the KRAS inhibitor comprises MCP110. In some embodiments, the KRAS inhibitor comprises 6H05. In some embodiments, the KRAS inhibitor comprises K-Ras(G12C) inhibitor 12. In some embodiments, the KRAS inhibitor comprises Kobe0065. In some embodiments, the KRAS inhibitor comprises K-Ras(G12C) inhibitor 6. In some embodiments, the KRAS inhibitor comprises BQU57. In some embodiments, the KRAS inhibitor comprises ARS-1620. In some embodiments, the KRAS inhibitor comprises JDQ443. In some embodiments, the KRAS inhibitor comprises Kobe2602. In some embodiments, the KRAS inhibitor comprises BI-2852. In some embodiments, the KRAS inhibitor comprises NAV-2729. In some embodiments, the KRAS inhibitor comprises antineoplaston A10. In some embodiments, the KRAS inhibitor comprises fendiline hydrochloride. In some embodiments, the KRAS inhibitor comprises adagrasib. In some embodiments, the KRAS inhibitor comprises KRpep-2d. In some embodiments, the KRAS inhibitor comprises ARS-853. In some embodiments, the KRAS inhibitor comprises BI-3406. In some embodiments, the KRAS inhibitor comprises perillyl alcohol. In some embodiments, the KRAS inhibitor comprises ASP2453. In some embodiments, the KRAS inhibitor comprises RBC8. In some embodiments, the KRAS inhibitor comprises sotorasib racemate. In some embodiments, the KRAS inhibitor comprises Pan-RAS-IN-1. In some embodiments, the KRAS inhibitor comprises MRTX-1257. In some embodiments, the KRAS inhibitor comprises KY1220. In some embodiments, the KRAS inhibitor comprises CID-1067700. In some embodiments, the KRAS inhibitor comprises zoledronic acid monohydrate. In some embodiments, the KRAS inhibitor comprises D-1553. In some embodiments, the KRAS inhibitor comprises RG6330/GDC-6036. In some embodiments, the KRAS inhibitor comprises BI-1823911. In some embodiments, the KRAS inhibitor comprises JAB-21822. In some embodiments, the KRAS inhibitor comprises LY3537982. In some embodiments, the KRAS inhibitor comprises JNJ-74699157. In some embodiments, the KRAS inhibitor comprises MK-1084. In some embodiments, the KRAS inhibitor comprises JAB-22000. In some embodiments, the KRAS inhibitor comprises JAB-23000. In some embodiments, the KRAS inhibitor comprises RM-018. In some embodiments, the KRAS inhibitor comprises RMC-6291. In some embodiments, the KRAS inhibitor comprises RMC-9805/RM-036. In some embodiments, the KRAS inhibitor comprises RMC-8839. In some embodiments, the KRAS inhibitor comprises RMC-6236. In some embodiments, the KRAS inhibitor comprises JAB-23400. In some embodiments, the KRAS inhibitor comprises BBP-454.

In some embodiments, the subject can also be administered an AKT inhibitor. In some embodiments, the AKT inhibitor comprises MK-2206 2HCl, perifosine (e.g., KRX-0401), GSK690693, rigosertib (e.g., ON-01910), ipatasertib (e.g., GDC-0068), capivasertib (e.g., AZD5363), PF-04691502, AT7867, triciribine (e.g., NSC 154020), CCT128930, A-674563, PHT-427, miltefosine, honokiol (e.g., NSC 293100), PI3K/Akt Inhibitor library, TIC10 analogue, demethyl-coclaurine, oroxin B, homosalate, daphnoretin, rotundic acid, cinobufagin, neferine, alobresib (e.g., GS-5829), borussertib, resibufogenin, hispidulin, lanatoside C, astragaloside IV, zeaxanthin, A-443654, uprosertib (e.g., GSK2141795), TIC10 (e.g., ONC201), akti-1/2, SC66, Dichroa febrifuga extract, MAZ51, usnic acid, Dioscoreae nipponicae Rhizoma extract, Cinnamomi ramulus extract, miransertib (e.g., ARQ 092) HCl, lupenone, miransertib (e.g., ARQ-092), (E)-Akt inhibitor-IV, notoginsenoside R1, Alpiniae Katsumadai extract, afuresertib (e.g., SK2110183), praeruptorin A, Weigela Grandiflora Fortune extract, LM22B-10, solasodine, pectolinarin, α-Linolenic acid, BIA, ailanthone, methyl-hesperidin, RPI-1, BAY1125976, oridonin (e.g., NSC-250682), hematein, scutellarin, ABTL-0812, ML-9 HCl, SPP-86, AT13148, urolithin B, trigonelline, deguelin, SC79, amarogentin, loureirin A, or YS-49, or any combination thereof. In some embodiments, the AKT inhibitor comprises MK-2206 2HCl. In some embodiments, the AKT inhibitor comprises perifosine. In some embodiments, the AKT inhibitor comprises GSK690693. In some embodiments, the AKT inhibitor comprises rigosertib. In some embodiments, the AKT inhibitor comprises ipatasertib. In some embodiments, the AKT inhibitor comprises capivasertib. In some embodiments, the AKT inhibitor comprises PF-04691502. In some embodiments, the AKT inhibitor comprises AT7867. In some embodiments, the AKT inhibitor comprises triciribine. In some embodiments, the AKT inhibitor comprises CCT128930. In some embodiments, the AKT inhibitor comprises A-674563. In some embodiments, the AKT inhibitor comprises PHT-427. In some embodiments, the AKT inhibitor comprises miltefosine. In some embodiments, the AKT inhibitor comprises honokiol. In some embodiments, the AKT inhibitor comprises PI3K/Akt Inhibitor library. In some embodiments, the AKT inhibitor comprises TIC10 analogue. In some embodiments, the AKT inhibitor comprises demethyl-coclaurine. In some embodiments, the AKT inhibitor comprises oroxin B. In some embodiments, the AKT inhibitor comprises homosalate. In some embodiments, the AKT inhibitor comprises daphnoretin. In some embodiments, the AKT inhibitor comprises rotundic acid. In some embodiments, the AKT inhibitor comprises cinobufagin. In some embodiments, the AKT inhibitor comprises neferine. In some embodiments, the AKT inhibitor comprises alobresib. In some embodiments, the AKT inhibitor comprises borussertib. In some embodiments, the AKT inhibitor comprises resibufogenin. In some embodiments, the AKT inhibitor comprises hispidulin. In some embodiments, the AKT inhibitor comprises lanatoside C. In some embodiments, the AKT inhibitor comprises astragaloside IV. In some embodiments, the AKT inhibitor comprises zeaxanthin. In some embodiments, the AKT inhibitor comprises A-443654. In some embodiments, the AKT inhibitor comprises uprosertib. In some embodiments, the AKT inhibitor comprises TIC10. In some embodiments, the AKT inhibitor comprises akti-1/2. In some embodiments, the AKT inhibitor comprises SC66. In some embodiments, the AKT inhibitor comprises Dichroa febrifuga extract. In some embodiments, the AKT inhibitor comprises MAZ51. In some embodiments, the AKT inhibitor comprises usnic acid. In some embodiments, the AKT inhibitor comprises Dioscoreae nipponicae Rhizoma extract. In some embodiments, the AKT inhibitor comprises Cinnamomi ramulus extract. In some embodiments, the AKT inhibitor comprises miransertib HCl. In some embodiments, the AKT inhibitor comprises lupenone. In some embodiments, the AKT inhibitor comprises miransertib. In some embodiments, the AKT inhibitor comprises (E)-Akt inhibitor-IV. In some embodiments, the AKT inhibitor comprises notoginsenoside R1. In some embodiments, the AKT inhibitor comprises Alpiniae Katsumadai extract. In some embodiments, the AKT inhibitor comprises afuresertib. In some embodiments, the AKT inhibitor comprises praeruptorin A. In some embodiments, the AKT inhibitor comprises Weigela Grandiflora Fortune extract. In some embodiments, the AKT inhibitor comprises LM22B-10. In some embodiments, the AKT inhibitor comprises solasodine. In some embodiments, the AKT inhibitor comprises pectolinarin. In some embodiments, the AKT inhibitor comprises α-Linolenic acid. In some embodiments, the AKT inhibitor comprises BIA. In some embodiments, the AKT inhibitor comprises ailanthone. In some embodiments, the AKT inhibitor comprises methyl-hesperidin. In some embodiments, the AKT inhibitor comprises RPI-1. In some embodiments, the AKT inhibitor comprises BAY1125976. In some embodiments, the AKT inhibitor comprises oridonin. In some embodiments, the AKT inhibitor comprises hematein. In some embodiments, the AKT inhibitor comprises scutellarin. In some embodiments, the AKT inhibitor comprises ABTL-0812. In some embodiments, the AKT inhibitor comprises ML-9 HCl. In some embodiments, the AKT inhibitor comprises SPP-86. In some embodiments, the AKT inhibitor comprises AT13148. In some embodiments, the AKT inhibitor comprises urolithin B. In some embodiments, the AKT inhibitor comprises trigonelline. In some embodiments, the AKT inhibitor comprises deguelin. In some embodiments, the AKT inhibitor comprises SC79. In some embodiments, the AKT inhibitor comprises amarogentin. In some embodiments, the AKT inhibitor comprises loureirin A. In some embodiments, the AKT inhibitor comprises YS-49.

In some embodiments, the subject can also be administered an ERK inhibitor. In some embodiments, the ERK inhibitor comprises 3′-Hydroxypterostilbene, selumetinib (e.g., AZD6244), SU1498, SCH772984, alobresib (e.g., 5-5829), astragaloside IV, DC260126, senkyunolide I, ASN007, cucurbitacin IIb, mirdametinib (e.g., PD0325901), MK-8353 (e.g., SCH900353), AZD0364 (e.g., ATG-017), usnic acid, ezatiostat, SL-327, BM213, 3-Deazaadenosine hydrochloride, DMU-212, CGP 57380, notoginsenoside R1, NLX-204, CC-90003, honokiol (e.g., NSC 293100), 1-191, KO-947, doramapimod (e.g., BIRB 796), WP1066, tanzisertib (e.g., CC-930), 2′,5′-Dihydroxyacetophenone, trametinib (e.g., GSK1120212), PD98059, magnolin, PLX7904, STAT5-IN-2, temuterkib (e.g., LY3214996), pluripotin (e.g., SC1), ulixertinib (e.g., BVD-523), methylthiouracil, pamoic acid disodium, FR 180204, AG-126, NSC87877, corynoxeine, TAK-715, agerafenib (e.g., RXDX-105), NSC95397, tracheloside, or ravoxertinib (e.g., GDC-0994), or any combination thereof. In some embodiments, the ERK inhibitor comprises 3′-Hydroxypterostilbene. In some embodiments, the ERK inhibitor comprises selumetinib. In some embodiments, the ERK inhibitor comprises SU1498. In some embodiments, the ERK inhibitor comprises SCH772984. In some embodiments, the ERK inhibitor comprises alobresib. In some embodiments, the ERK inhibitor comprises astragaloside IV. In some embodiments, the ERK inhibitor comprises DC260126. In some embodiments, the ERK inhibitor comprises senkyunolide I. In some embodiments, the ERK inhibitor comprises ASN007. In some embodiments, the ERK inhibitor comprises cucurbitacin IIb. In some embodiments, the ERK inhibitor comprises mirdametinib. In some embodiments, the ERK inhibitor comprises MK-8353. In some embodiments, the ERK inhibitor comprises AZD0364. In some embodiments, the ERK inhibitor comprises usnic acid. In some embodiments, the ERK inhibitor comprises ezatiostat. In some embodiments, the ERK inhibitor comprises SL-327. In some embodiments, the ERK inhibitor comprises BM213. In some embodiments, the ERK inhibitor comprises 3-Deazaadenosine hydrochloride. In some embodiments, the ERK inhibitor comprises DMU-212. In some embodiments, the ERK inhibitor comprises CGP 57380. In some embodiments, the ERK inhibitor comprises notoginsenoside R1. In some embodiments, the ERK inhibitor comprises NLX-204. In some embodiments, the ERK inhibitor comprises CC-90003. In some embodiments, the ERK inhibitor comprises honokiol. In some embodiments, the ERK inhibitor comprises I-191. In some embodiments, the ERK inhibitor comprises KO-947. In some embodiments, the ERK inhibitor comprises doramapimod. In some embodiments, the ERK inhibitor comprises WP1066. In some embodiments, the ERK inhibitor comprises tanzisertib. In some embodiments, the ERK inhibitor comprises 2′,5′-Dihydroxyacetophenone. In some embodiments, the ERK inhibitor comprises trametinib. In some embodiments, the ERK inhibitor comprises PD98059. In some embodiments, the ERK inhibitor comprises magnolin. In some embodiments, the ERK inhibitor comprises PLX7904. In some embodiments, the ERK inhibitor comprises STAT5-IN-2. In some embodiments, the ERK inhibitor comprises temuterkib. In some embodiments, the ERK inhibitor comprises pluripotin. In some embodiments, the ERK inhibitor comprises ulixertinib. In some embodiments, the ERK inhibitor comprises methylthiouracil. In some embodiments, the ERK inhibitor comprises pamoic acid disodium. In some embodiments, the ERK inhibitor comprises FR 180204. In some embodiments, the ERK inhibitor comprises AG-126. In some embodiments, the ERK inhibitor comprises NSC87877. In some embodiments, the ERK inhibitor comprises corynoxeine. In some embodiments, the ERK inhibitor comprises TAK-715. In some embodiments, the ERK inhibitor comprises agerafenib. In some embodiments, the ERK inhibitor comprises NSC95397. In some embodiments, the ERK inhibitor comprises tracheloside. In some embodiments, the ERK inhibitor comprises ravoxertinib.

In some embodiments, the subject can also be administered an ERBB inhibitor. In some embodiments, the ERBB inhibitor comprises erlotinib (e.g., OSI-774) HCl, gefitinib (e.g., ZD1839), lapatinib (e.g., GW-572016), ditosylate, afatinib (e.g., BIBW2992), saracatinib (e.g., AZD0530), vandetanib (e.g., ZD6474), neratinib (e.g., HKI-272), canertinib (e.g., CI-1033), lapatinib (e.g., GW-572016), AG-490 (e.g., Tyrphostin B42), CP-724714, dacomitinib (e.g., PF-00299804), WZ4002, sapitinib (e.g., AZD8931), CUDC-101, AG-1478 (e.g., Tyrphostin AG-1478), PD153035 HCl, pelitinib (e.g., EKB-569), AC480 (e.g., BMS-599626), AEE788 (e.g., NVP-AEE788), AP26113-analog (e.g., ALK-IN-1), OSI-420, WZ3146, HER2-Inhibitor-1, WZ8040, allitinib tosylate, rociletinib (e.g., CO-1686), genistein (e.g., NPI 031L), varlitinib, icotinib (e.g., BPI-2009H), TAK-285, WHI-P154, daphnetin, PD168393, tyrphostin 9, CNX-2006, AG-18, licochalcone D, canertinib dihydrochloride, AG 555, BLU-945, CH7233163, AZ5104, lazertinib, osimertinib (e.g., AZD9291), CL-387785 (e.g., EKI-785), tyrphostin AG-528, AG 494, AG 556, tucatinib, olmutinib (e.g., BI 1482694), necitumumab (anti-EGFR), RG14620, panitumumab (anti-EGFR), PD153035, EBE-A22, MTX-211, mobocertinib (e.g., TAK788), (−)-Epigallocatechin Gallate, erlotinib (e.g., OSI-774), gefitinib-based PROTAC 3, gefitinib hydrochloride, tyrphostin AG30 (e.g., AG30), alflutinib (e.g., AST2818) mesylate, afatinib (e.g., BIBW2992) Dimaleate, zorifertinib (e.g., AZD3759), ErbB2 inhibitor, poziotinib (e.g., HM781-36B), brigatinib (e.g., AP26113), osimertinib mesylate, TQB3804 (e.g., EGFR-IN-7), EGFR Inhibitor, naquotinib (e.g., ASP8273), chrysophanic acid, kaempferide, AV-412 free base, nazartinib (e.g., EGF816), AST-1306, (Rac)-JBJ-04-125-02, JND3229, BI-4020, norcantharidin, AG99, theliatinib (e.g., HMPL-309), AG-1557, BDTX-189, RG 13022, lifirafenib (e.g., BGB-283), lidocaine hydrochloride, sunvozertinib, cetuximab (anti-EGFR), falnidamol, pyrotinib (e.g., SHR-1258) dimaleate, 0-Demethyl-Gefitinib, butein, EAI045, NSC228155, zipalertinib, cyasterone, epertinib hydrochloride, SU5214, almonertinib (e.g., HS-10296), avitinib (e.g., AC0010), EGF Rabbit Recombinant mAb, herceptin, or trastuzumab, or any combination thereof. In some embodiments, the ERBB inhibitor comprises erlotinib HCl. In some embodiments, the ERBB inhibitor comprises gefitinib. In some embodiments, the ERBB inhibitor comprises lapatinib. In some embodiments, the ERBB inhibitor comprises ditosylate. In some embodiments, the ERBB inhibitor comprises afatinib. In some embodiments, the ERBB inhibitor comprises saracatinib. In some embodiments, the ERBB inhibitor comprises vandetanib. In some embodiments, the ERBB inhibitor comprises neratinib. In some embodiments, the ERBB inhibitor comprises canertinib. In some embodiments, the ERBB inhibitor comprises lapatinib. In some embodiments, the ERBB inhibitor comprises AG-490. In some embodiments, the ERBB inhibitor comprises CP-724714. In some embodiments, the ERBB inhibitor comprises dacomitinib. In some embodiments, the ERBB inhibitor comprises WZ4002. In some embodiments, the ERBB inhibitor comprises sapitinib. In some embodiments, the ERBB inhibitor comprises CUDC-101. In some embodiments, the ERBB inhibitor comprises AG-1478. In some embodiments, the ERBB inhibitor comprises PD153035 HCl. In some embodiments, the ERBB inhibitor comprises pelitinib. In some embodiments, the ERBB inhibitor comprises AC480. In some embodiments, the ERBB inhibitor comprises AEE788. In some embodiments, the ERBB inhibitor comprises AP26113-analog. In some embodiments, the ERBB inhibitor comprises OSI-420. In some embodiments, the ERBB inhibitor comprises WZ3146. In some embodiments, the ERBB inhibitor comprises HER2-Inhibitor-1. In some embodiments, the ERBB inhibitor comprises WZ8040. In some embodiments, the ERBB inhibitor comprises allitinib tosylate. In some embodiments, the ERBB inhibitor comprises rociletinib. In some embodiments, the ERBB inhibitor comprises genistein. In some embodiments, the ERBB inhibitor comprises varlitinib. In some embodiments, the ERBB inhibitor comprises icotinib. In some embodiments, the ERBB inhibitor comprises TAK-285. In some embodiments, the ERBB inhibitor comprises WHI-P154. In some embodiments, the ERBB inhibitor comprises daphnetin. In some embodiments, the ERBB inhibitor comprises PD168393. In some embodiments, the ERBB inhibitor comprises tyrphostin 9. In some embodiments, the ERBB inhibitor comprises CNX-2006. In some embodiments, the ERBB inhibitor comprises AG-18. In some embodiments, the ERBB inhibitor comprises licochalcone D. In some embodiments, the ERBB inhibitor comprises canertinib dihydrochloride. In some embodiments, the ERBB inhibitor comprises AG 555. In some embodiments, the ERBB inhibitor comprises BLU-945. In some embodiments, the ERBB inhibitor comprises CH7233163. In some embodiments, the ERBB inhibitor comprises AZ5104. In some embodiments, the ERBB inhibitor comprises Lazertinib. In some embodiments, the ERBB inhibitor comprises Osimertinib. In some embodiments, the ERBB inhibitor comprises CL-387785. In some embodiments, the ERBB inhibitor comprises tyrphostin AG-528. In some embodiments, the ERBB inhibitor comprises AG 494. In some embodiments, the ERBB inhibitor comprises AG 556. In some embodiments, the ERBB inhibitor comprises tucatinib. In some embodiments, the ERBB inhibitor comprises olmutinib. In some embodiments, the ERBB inhibitor comprises necitumumab (anti-EGFR). In some embodiments, the ERBB inhibitor comprises RG14620. In some embodiments, the ERBB inhibitor comprises panitumumab (anti-EGFR). In some embodiments, the ERBB inhibitor comprises PD153035. In some embodiments, the ERBB inhibitor comprises EBE-A22. In some embodiments, the ERBB inhibitor comprises MTX-211. In some embodiments, the ERBB inhibitor comprises mobocertinib. In some embodiments, the ERBB inhibitor comprises (−)-Epigallocatechin Gallate. In some embodiments, the ERBB inhibitor comprises erlotinib. In some embodiments, the ERBB inhibitor comprises gefitinib-based PROTAC 3. In some embodiments, the ERBB inhibitor comprises gefitinib hydrochloride. In some embodiments, the ERBB inhibitor comprises tyrphostin AG30. In some embodiments, the ERBB inhibitor comprises alflutinib mesylate. In some embodiments, the ERBB inhibitor comprises afatinib dimaleate. In some embodiments, the ERBB inhibitor comprises zorifertinib. In some embodiments, the ERBB inhibitor comprises ErbB2 inhibitor. In some embodiments, the ERBB inhibitor comprises poziotinib. In some embodiments, the ERBB inhibitor comprises brigatinib. In some embodiments, the ERBB inhibitor comprises osimertinib mesylate. In some embodiments, the ERBB inhibitor comprises TQB3804. In some embodiments, the ERBB inhibitor comprises EGFR inhibitor. In some embodiments, the ERBB inhibitor comprises naquotinib. In some embodiments, the ERBB inhibitor comprises chrysophanic acid. In some embodiments, the ERBB inhibitor comprises kaempferide. In some embodiments, the ERBB inhibitor comprises AV-412 free base. In some embodiments, the ERBB inhibitor comprises nazartinib. In some embodiments, the ERBB inhibitor comprises AST-1306. In some embodiments, the ERBB inhibitor comprises (Rac)-JBJ-04-125-02. In some embodiments, the ERBB inhibitor comprises JND3229. In some embodiments, the ERBB inhibitor comprises BI-4020. In some embodiments, the ERBB inhibitor comprises norcantharidin. In some embodiments, the ERBB inhibitor comprises AG99. In some embodiments, the ERBB inhibitor comprises theliatinib. In some embodiments, the ERBB inhibitor comprises AG-1557. In some embodiments, the ERBB inhibitor comprises BDTX-189. In some embodiments, the ERBB inhibitor comprises RG 13022. In some embodiments, the ERBB inhibitor comprises lifirafenib. In some embodiments, the ERBB inhibitor comprises lidocaine hydrochloride. In some embodiments, the ERBB inhibitor comprises sunvozertinib. In some embodiments, the ERBB inhibitor comprises cetuximab (anti-EGFR). In some embodiments, the ERBB inhibitor comprises falnidamol. In some embodiments, the ERBB inhibitor comprises pyrotinib dimaleate. In some embodiments, the ERBB inhibitor comprises O-Demethyl-Gefitinib. In some embodiments, the ERBB inhibitor comprises butein. In some embodiments, the ERBB inhibitor comprises EAI045. In some embodiments, the ERBB inhibitor comprises NSC228155. In some embodiments, the ERBB inhibitor comprises zipalertinib. In some embodiments, the ERBB inhibitor comprises cyasterone. In some embodiments, the ERBB inhibitor comprises epertinib hydrochloride. In some embodiments, the ERBB inhibitor comprises SU5214. In some embodiments, the ERBB inhibitor comprises almonertinib. In some embodiments, the ERBB inhibitor comprises avitinib. In some embodiments, the ERBB inhibitor comprises EGF Rabbit Recombinant mAb. In some embodiments, the ERBB inhibitor comprises Herceptin. In some embodiments, the ERBB inhibitor comprises trastuzumab.

In some embodiments, the subject can also be administered an ABCB1(MDR1) inhibitor. In some embodiments, the ABCB1(MDR1) inhibitor comprises any one or more of the following: afatinib, dacomitinib, dovitinib, erdafitinib, erlotinib, everolimus, gefitinib, imatinib, lapatinib, neratinib, nintedanib, ponatinib, regorafenib, sirolimus, sorafenib, sunitinib, temsirolimus, vemurafenib, osimertinib, pelitinib, WZ3146, WZ4002, deforolimus, rapamycin, WYE-687, WAY-600, BEZ235, cabozantinib, canertinib, pazopanib, icotinib, abemaciclib, alectinib, avapritinib, axitinib, bosutinib, crizotinib, entrectinib, fedratinib, idelalisib, lenvatinib, mobocertinib, nilotinib, palbociclib, ripretinib, selpercatinib, sotorasib, tepotinib, tucatinib, umbralisib, upadacitinib, vandetanib, fostamatinib, voruciclib, purvalanol A, olomoucine II, roscovitine, NVP-TAE684, SNS-314, LY2603618, MP-470, masitinib, Ki8751, BIBW2992, TG101209, CI-1033, NVP-ADW742, NVP-BSK805, crenolanib, BI2536, GSK461364, BI6727, AZD0530, cediranib, AV-951, ceritinib, motesanib, saracatinib, vatalanib, apatinib, dasatinib, ibrutinib, linsitinib, quizartinib, tandutinib, vatalinib, telatinib, acetaminophen, alfentanil, alpelisib, amiodarone, amlodipine, amodiaquine, amoxapine, amsacrine, annamycin, arsenic trioxide, astemizole, asunaprevir, atazanavir, atorvastatin, atovaquone, azelastine, azilsartan medoxomil, azithromycin, belumosudil, benzocaine, benzquinamide, bepridil, berotralstat, bicalutamide, biricodar, bisoprolol, boceprevir, brefeldin A, bromocriptine, buprenorphine, buspirone, cabazitaxel, canagliflozin, candesartan, candesartan cilexetil, capmatinib, captopril, carfilzomib, carvedilol, caspofungin, ceftriaxone, cethromycin, cetirizine, chloroquine, chlorpromazine, chlorprothixene, cholesterol, cilazapril, citalopram, clarithromycin, clofazimine, clomipramine, clotrimazole, cobicistat, colforsin, concanamycin A, conivaptan, curcumin, cyclosporine, daclatasvir, dactinomycin, darunavir, daunorubicin, desipramine, desloratadine, desmethylsertraline, dexamethasone, dexamethasone acetate, dexniguldipine, dexverapamil, digoxin, dihydroergotamine, diltiazem, diosmin, dipyridamole, dofequidar, doxazosin, doxorubicin, dronabinol, dronedarone, duloxetine, econazole, elacridar, elagolix, elbasvir, elexacaftor, eliglustat, emopamil, enalapril, enasidenib, enzalutamide, ergometrine, ergotamine, erythromycin, esomeprazole, estramustine, etoposide, etravirine, favipiravir, felodipine, fenofibrate, fentanyl, fingolimod, flibanserin, fluconazole, fluoxetine, flupentixol, fluphenazine, flurazepam, fluvoxamine, galantamine, gallopamil, genistein, glasdegib, glecaprevir, glyburide, gramicidin D, grepafloxacin, haloperidol, HM-30181, hycanthone, hydroxychloroquine, ibuprofen, indinavir, indomethacin, infigratinib, isavuconazole, isavuconazonium, isradipine, istradefylline, itraconazole, ivacaftor, ivermectin, ivosidenib, ixabepilone, ketoconazole, lamotrigine, laniquidar, lansoprazole, lasmiditan, ledipasvir, letermovir, levofloxacin, levoketoconazole, lidocaine, linagliptin, lomerizine, lomitapide, lonafarnib, lopinavir, loratadine, losartan, lovastatin, loxapine, lumacaftor, lurbinectedin, medroxyprogesterone acetate, mefloquine, megestrol acetate, methadone, methylene blue, metronidazole, mibefradil, miconazole, mifepristone, mirabegron, mitotane, mitoxantrone, monensin, naproxen, nefazodone, nelfinavir, netupitant, nicardipine, nifedipine, niguldipine, nimodipine, nisoldipine, nitrendipine, norethisterone, norgestimate, olaparib, omeprazole, ONT-093, paclitaxel, paliperidone, pantoprazole, paritaprevir, paroxetine, pemigatinib, pibrentasvir, pimozide, piperine, polyethylene glycol, polyethylene glycol 400, posaconazole, pralsetinib, prazosin, prednisone, primaquine, progesterone, promethazine, propafenone, propranolol, protriptyline, quercetin, quinacrine, quinidine, quinine, ranitidine, ranolazine, reboxetine, relugolix, reserpine, reversin 121, rifamycin, rilpivirine, ritonavir, rolapitant, rucaparib, salinomycin, sapropterin, saquinavir, sarecycline, selegiline, sertraline, sildenafil, simeprevir, simvastatin, staurosporine, suvorexant, tacrolimus, tamoxifen, tariquidar, taurocholic acid, telaprevir, telmisartan, tenofovir disoproxil, terazosin, terfenadine, tesmilifene, testosterone, testosterone cypionate, testosterone enanthate, testosterone undecanoate, tetrandrine, tezacaftor, ticagrelor, tipifarnib, tipranavir, tolvaptan, toremifene, trifluoperazine, triflupromazine, trimethoprim, troleandomycin, valinomycin, valspodar, vardenafil, velpatasvir, venetoclax, venlafaxine, verapamil, vinblastine, vincristine, voacamine, voclosporin, vorapaxar, voxilaprevir, yohimbine, zimelidine, zonisamide, zosuquidar, elagolix-estradiol norethindrone, elexacaftor-tezacaftor-ivacaftor, ombitasvir-paritaprevir-ritonavir (technivie), ritonavir and ritonavir-containing coformulations, tamoxifen, tezacaftor-ivacaftor, glecaprevir-pibrentasvir, valspdar, encequidar, XR9051, YS-370, or any combination thereof.

In some embodiments, the subject can also be administered an RTK inhibitor. In some embodiments, the RTK inhibitor is erlotinib, afatinib, panitumumab, or cetuximab, or any combination thereof. In some embodiments, the RTK inhibitor is erlotinib. In some embodiments, the RTK inhibitor is afatinib. In some embodiments, the RTK inhibitor is panitumumab. In some embodiments, the RTK inhibitor is cetuximab.

In some embodiments, the subject can also be administered an SOS1 inhibitor. In some embodiments, the SOS1 inhibitor is BI-1701963, BI-3406, RMC-5845, or BAY-293, or any combination thereof. In some embodiments, the SOS1 inhibitor is BI-1701963. In some embodiments, the SOS1 inhibitor is BI-3406. In some embodiments, the SOS1 inhibitor is RMC-5845. In some embodiments, the SOS1 inhibitor is BAY-293.

In some embodiments, the subject can also be administered a CDK4/6 inhibitor. In some embodiments, the CDK4/6 inhibitor is palbociclib, abemaciclib, or ribociclib, or any combination thereof. In some embodiments, the CDK4/6 inhibitor is Palbociclib. In some embodiments, the CDK4/6 inhibitor is abemaciclib. In some embodiments, the CDK4/6 inhibitor is ribociclib.

In some embodiments, the subject can also be administered an AURKA inhibitor. In some embodiments, the AURKA inhibitor is VIC-1911.

In some embodiments, the subject can also be administered a WEE1 inhibitor. In some embodiments, the WEE1 inhibitor is adavosertib or LY3295668, or a combination thereof. In some embodiments, the WEE1 inhibitor is adavosertib. In some embodiments, the WEE1 inhibitor is LY3295668.

In some embodiments, the subject can also be administered an siRNA. In some embodiments, the siRNA comprises iExosomes.

In some embodiments, the subject can also be administered a cancer vaccine. In some embodiments, the cancer vaccine is mRNA-5671/V941, ELI-002, or mDC3/8-KRAS vaccine, or any combination thereof. In some embodiments, the cancer vaccine is mRNA-5671/V941. In some embodiments, the cancer vaccine is ELI-002. In some embodiments, the cancer vaccine is mDC3/8-KRAS vaccine.

Examples of anti-cancer therapeutic agents that may be used in any of the methods described herein include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antiobiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafarnib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives thereof. Also included are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives thereof.

Suitable anti-cancer agents comprise commercially or clinically available compounds such as TARCEVA® (erlotinib), TAXOTERE® (docetaxel), 5-FU (fluorouracil, 5-fluorouracil, CAS No. 51-21-8), GEMZAR® (gemcitabine), PD-0325901 (CAS No. 391210-10-9), cisplatin (cis-diamine, dichloroplatinum(II), CAS No. 15663-27-1), carboplatin (CAS No. 41575-94-4), TAXOL® (paclitaxel), HERCEPTIN® (trastuzumab), TEMODAR® and TEMODAL® (temozolomide; 4-methyl-5-oxo-2,3,4,6,8-pentazabicyclo [4.3.0] nona-2,7,9-triene-9-carboxamide, CAS No. 85622-93-1), NOLVADEX®, ISTUBAL®, and VALODEX® (tamoxifen; (Z)-2-[4-(1,2-diphenylbut-1-enyl)phenoxy]-N,N-dimethylethanamine), and ADRIAMYCIN® (doxorubicin). Additional commercially or clinically available anti-cancer agents comprise ELOXATIN® (oxaliplatin), VELCADE® (bortezomib), SUNITINIB® (sutent; SU11248), FEMARA® (letrozole), GLEEVEC® (imatinib mesylate), XL-518 (Mek inhibitor, WO 2007/044515), ARRY-886 (Mek inhibitor, AZD6244), SF-1126 (PI3K inhibitor), BEZ-235 (PI3K inhibitor), XL-147 (PI3K inhibitor), PTK787/ZK 222584, FASLODEX® (fulvestrant), leucovorin (folinic acid), RAPAMUNE® (rapamycin; sirolimus), TYKERB® (lapatinib; GSK572016), SARASAR™ (lonafarnib, SCH 66336), NEXAVAR® (sorafenib; BAY43-9006), IRESSA® (gefitinib), CAMPTOSAR® (irinotecan; CPT-11), ZARNESTRA™ (tipifarnib), ABRAXANE™ (Cremophor-free), albumin-engineered nanoparticle formulations of paclitaxel, ZACTIMA® (vandetanib; rINN, ZD6474,), chloranmbucil, AG1478, AG1571 (SU 5271), TORISEL® (temsirolimus), pazopanib, TELCYTA® (canfosfamide), CYTOXAN® (thiotepa) and NEOSAR® (cyclosphosphamide); NAVELBINE® (vinorelbine); XELODA® (capecitabine), NOLVADEX® (tamoxifen); FARESTON® (tamoxifen citrate), MEGASE® (megestrol acetate), AROMASIN® (exemestane), formestanie, fadrozole, RIVISOR® (vorozole), FEMARA® (letrozole), and ARIMIDEX® (anastrozole).

Particularly suitable anti-cancer chemotherapeutic agents include, but are not limited to, Mechlorethamine hydrochloride, Cyclophosphamide, Ifosfamide, Chlorambucil, Melphalan, Busulfan, Thiotepa (Triethylenethiophosphoramide), Carmustine, Lomustine, Streptozocin, Vincristine, Vinblastine, Paclitaxel, Methotrexate, Mercaptopurine, Thioguanine, Fluorouracil, Cytarabine, Azacitidine, Dactinomycin, Doxorubicin, Daunorubicin, Idarubicin, Bleomycin, Picamycin, Mitomycin, Hydroxyurea, Procarbazine, Dacarbazine, Cisplatin, Carboplatin, Asparaginase, Etoposide, Amsarcrine, Mitotane, Shreptozoin, Altretamine, Teniposde, Plcamydin, Fluorodeoxyuridine, CB3717, Floxuridine, Pentostatin, Cyctrabine, Fludarabine, Irinotecan, Adriamycin, Camptothecin, α-, β-, or γ-Interferon, Interleukin-2, Docetaxel, Topotecan, and Mitoxantrone, etc., or adjuvant therapies that further stimulate the immune response.

In some embodiments, the subject is a non-responder or resistant to treatment with an MEK inhibitor. In some embodiments, the subject is a non-responder or resistant to treatment with a RAS inhibitor. In some embodiments, the subject is a non-responder or resistant to treatment with a KRAS inhibitor. In some embodiments, the subject is a non-responder or resistant to treatment with a RAF inhibitor. In some embodiments, the subject is a non-responder or resistant to treatment with an ERK inhibitor. In some embodiments, the subject is a non-responder or resistant to treatment with a PI3K inhibitor. In some embodiments, the subject is a non-responder or resistant to treatment with an AKT inhibitor. In some embodiments, the subject is a non-responder or resistant to treatment with an MTOR inhibitor. In some embodiments, the subject is a non-responder or resistant to treatment with a BET bromodomain inhibitor. In some embodiments, the subject is a non-responder or resistant to treatment with an SHP2 inhibitor. In some embodiments, the subject is a non-responder or resistant to treatment with an SOS inhibitor. In some embodiments, the subject is a non-responder or resistant to treatment with an RTK inhibitor.

In some embodiments, the pharmaceutical composition is an oral dosage formulation, an intravenous dosage formulation, a topical dosage formulation, an intraperitoneal dosage formulation, or an intrathecal dosage form. In some embodiments, the pharmaceutical composition is an oral dosage formulation. In some embodiments, the pharmaceutical composition is an intravenous dosage formulation. In some embodiments, the pharmaceutical composition is a topical dosage formulation. In some embodiments, the pharmaceutical composition is an intraperitoneal dosage formulation. In some embodiments, the pharmaceutical composition is an intrathecal dosage form.

In some embodiments, the pharmaceutical composition is an oral dosage formulation in the form of a pill, tablet, capsule, cachet, gel-cap, pellet, powder, granule, or liquid.

In some embodiments, the pharmaceutical composition the oral dosage formulation is protected from light and present within a blister pack, bottle, or intravenous bag.

The compounds and compositions described herein can be formulated for parenteral administration by injection, such as by bolus injection or continuous infusion. The compounds and compositions can be administered by continuous infusion subcutaneously over a period of about 15 minutes to about 24 hours. The compositions can take such forms as suspensions, solutions or emulsions in oily or aqueous vehicles, and can contain formulary agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds and compositions described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds and compositions described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

In transdermal administration, the compounds and compositions can be applied to a plaster, or can be applied by transdermal, therapeutic systems that are consequently supplied to the organism. In some embodiments, the compounds and compositions are present in creams, solutions, powders, fluid emulsions, fluid suspensions, semi-solids, ointments, pastes, gels, jellies, and foams, or in patches containing any of the same.

The compounds and compositions described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds and compositions can be formulated with suitable polymeric or hydrophobic materials (for example as an emulsion in an acceptable oil) or ion exchange resins, or as sparingly soluble derivatives, for example, as a sparingly soluble salt.

In some embodiments, the compounds and compositions can be delivered in a controlled release system. In some embodiments, a pump may be used (see Langer, supra; Sefton, CRC Crit. Ref Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In some embodiments, a controlled-release system can be placed in proximity of the target of the compounds and compositions described herein, such as the liver, thus requiring a fraction of the systemic dose (see, e.g., Goodson, in Medical Applications of Controlled Release, supra, vol. 2, pp. 115-138 (1984)). Other controlled-release systems discussed in the review by Langer, Science, 1990, 249, 1527-1533) may be used.

The compounds and compositions described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

In some embodiments, the compounds and compositions described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

The amount of compound to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances. In some embodiments, the amount of any of the compounds described herein to be administered to a subject can be from about 1 mg to about 1000 mg, from about 10 mg to about 900 mg, from about 50 mg to about 750 mg, from about 100 mg to about 500 mg, from about 200 mg to about 400 mg, or from about 250 mg to about 350 mg.

The compounds and compositions described herein can be administered by any route of administration including, but not limited to, oral, sublingual, buccal, rectal, intranasal, inhalation, eye drops, ear drops, epidural, intracerebral, intracerebroventricular, intrathecal, epicutaneous or transdermal, subcutaneous, intradermal, intravenous, intraarterial, intraosseous infusion, intramuscular, intracardiac, intraperitoneal, intravesical infusion, and intravitreal. In some embodiments, the administration is oral, sublingual, buccal, rectal, intranasal, inhalation, eye drops, or ear drops. In some embodiments, the administration is oral, sublingual, buccal, rectal, intranasal, or inhalation. In some embodiments, the administration is epidural, intracerebral, intracerebroventricular, or intrathecal. In some embodiments, the administration is epicutaneous or transdermal, subcutaneous, or intradermal. In some embodiments, the administration is intravenous, intraarterial, intraosseous infusion, intramuscular, intracardiac, intraperitoneal, intravesical infusion, or intravitreal. In some embodiments, the administration is intravenous, intramuscular, or intraperitoneal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, or compositions comprising the same, to a particular area in need of treatment. This may be achieved, for example, by local infusion (for example, during surgery), topical application (for example, with a wound dressing after surgery), by injection (for example, by depot injection), catheterization, by suppository, or by an implant (for example, where the implant is of a porous, non-porous, or gelatinous material, including membranes, such as silastic membranes, or fibers). Formulations for injection can be presented in unit dosage form, such as in ampoules or in multi-dose containers, with an added preservative.

The present disclosure also provides dual RAF/MEK degraders for use in treating human diseases that require RAF-MEK. The dual RAF/MEK degraders can be any of the dual RAF/MEK degraders described herein. The human diseases that require RAF-MEK can be any of the human diseases that require RAF-MEK described herein.

The present disclosure also provides dual RAF/MEK degraders for use in the preparation of a medicament for treating human diseases that require RAF-MEK. The dual RAF/MEK degraders can be any of the dual RAF/MEK degraders described herein. The human diseases that require RAF-MEK can be any of the human diseases that require RAF-MEK described herein.

Disclosed herein are method of treating a cancer in a subject. In some aspects, the methods can comprise administering a therapeutically effective amount of a MEK1/2 degrader and a therapeutically effective amount of a BCL-2 inhibitor to a subject in need thereof, thereby treating the cancer in the subject. In some aspects, the MEK1/2 degrader can be MS934. In some aspects, the BCL-2 inhibitor can be naritoclax, venetoclax, obatoclax, or oblimersen sodium.

Disclosed herein are methods of reducing tumor growth in a subject. In some aspects, the methods can comprise administering a therapeutically effective amount of a MEK1/2 degrader and a therapeutically effective amount of a BCL-2 inhibitor to a subject in need thereof, thereby reducing the tumor growth in the subject. In some aspects, the MEK1/2 degrader can be MS934. In some aspects, the BCL-2 inhibitor can be naritoclax, venetoclax, obatoclax, or oblimersen sodium.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: General Methodology

Cell culture and drug treatment. For short-term growth assays, 1000-5000 cells were plated per well in 96-well plates and allowed to adhere and equilibrate overnight. Drug was added the following morning and after 120 hours of drug treatment, cell viability was assessed using the CellTiter-Glo Luminescent cell viability assay according to the manufacturer's instructions (Promega). Samples were run in biological triplicates (N=3) and Student's t-tests were performed for statistical analyses and p values ≤0.05 were considered significant. For long term colony formation assays, cells were plated in 24-well dishes (1000-5000 cells per well) and incubated overnight before continuous drug treatment for 2 weeks, with drug and medium replenished twice weekly. Following the final treatment, cells were rinsed with PBS and fixed with chilled methanol for 10 minutes at −20° C. Methanol was removed by aspiration, and cells were stained with 0.5% crystal violet in 20% methanol for 1 hour at room temperature. Samples were run in biological triplicates (N=3).

Immunoblotting Quantitation. Samples were harvested in lysis buffer (50 mM HEPES (pH 7.5), 0.5% Triton X-100, 150 mM NaCl, 1 mM EDTA, 1 mM EGTA, 10 mM sodium fluoride, 2.5 mM sodium orthovanadate, 1× protease inhibitor cocktail (Roche), and 1% each of phosphatase inhibitor cocktails 2 and 3 (Sigma)). Particulate was removed by centrifugation of lysates at 21,000 rpm for 15 minutes at 4° C. Lysates were subjected to sodium dodecyl sulfate-polyacrylamide gel electrophoresis (SDS-PAGE) chromatography and transferred to PVDF membranes before western blotting with primary antibodies. Secondary HRP-anti-rabbit and HRP-anti-mouse were obtained from ThermoFisher Scientific. SuperSignal West Pico and Femto Chemiluminescent Substrates (Thermo) were used to visualize blots. Western blot images were quantified using the Analyze>Gels function in Image J open-source software (National Institutes of Health). Optical density values for total protein levels were normalized by GAPDH. DC50 (the concentration where 50% of protein has been degraded) plots and values were calculated using PRISM software. Samples were run in biological triplicates (N=3) and Student's t-tests were performed for statistical analyses and P values ≤0.05 were considered significant.

VHL MEK1/MEK2 knockdown. Small interfering RNAs (siRNA) transfections were performed using 25 nM siRNA duplex and the reverse transfection protocol. 3000-5000 cells per well were added to 96-well plates with media containing the VHL, MEK1, or MEK2 siRNA and transfection reagent (Lipofectamine RNAiMax) according to the manufacturer's instructions. Cells were allowed to grow for 120 hours after transfection prior to CellTiter-Glo (Promega) analysis. Two to three independent experiments were performed with each cell line and siRNA. Samples were run in biological triplicates (N=3) and Student's t-tests were performed for statistical analyses and P values ≤0.05 were considered significant. For Western blot (immunoblotting) studies, the same procedure was performed with volumes and cell numbers proportionally scaled to a 60 mm or 10 cm dish, and cells were collected 72 hours after transfection.

Proteomics/pathway analysis. Single Run Total Proteomics and Nano LC MS/MS: Parental or PROTAC-resistant cells were lysed in a buffer containing 50 mM HEPES pH 8.0+4% SDS, and 100 g of protein was digested using LysC for 3 hours and trypsin overnight. Digested peptides were isolated using C-18 and PGC columns, then dried and cleaned with ethyl acetate. Three g of proteolytic peptides were resuspended in 0.1% formic acid and separated with a Thermo Scientific RSLC nano Ultimate 3000 LC on a Thermo Scientific Easy-Spray C-18 PepMap 75 μm×50 cm C-18 2 μm column. A 305 minute gradient of 2-20% (180 minute) 20%-28% (45 minute) 28%-48% (20 minute) acetonitrile with 0.1% formic acid was run at 300 nL/min at 50° C. Eluted peptides were analyzed by Thermo Scientific Q Exactive or Q Exactive plus mass spectrometers utilizing a top 15 methodology in which the 15 most intense peptide precursor ions were subjected to fragmentation. The AGC for MS1 was set to 3×10⁶ with a max injection time of 120 ms, the AGC for MS2 ions was set to 1×10⁵ with a max injection time of 150 ms, and the dynamic exclusion was set to 90 s.

Proteomics Data Processing: Raw data analysis of LFQ experiments was performed using MaxQuant software 1.6.0.1 and searched using Andromeda 1.5.6.0 against the Swiss-Prot human protein database (downloaded on Apr. 24, 2019, 20402 entries). The search was set up for full tryptic peptides with a maximum of two missed cleavage sites. All settings were default and searched using acetylation of protein N-terminus and oxidized methionine as variable modifications. Carbamidomethylation of cysteine was set as fixed modification. The precursor mass tolerance threshold was set at 10 ppm and maximum fragment mass error was 0.02 Da. LFQ quantitation was performed using MaxQuant with the following parameters; LFQ minimum ratio count: Global parameters for protein quantitation were as follows: label minimum ratio count: 1, peptides used for quantitation: unique, use modified proteins selected and with normalized average ratio estimation selected. Match between runs was employed for LFQ quantitation and the significance threshold of the ion score was calculated based on a false discovery rate of <1%. MaxQuant normalized LFQ values were imported into Perseus software (1.6.2.3) and filtered in the following manner: Proteins identified by site were removed, reverse, or potential contaminant were removed then filtered for proteins identified by >1 unique peptide. Protein LFQ values were log 2 transformed, filtered for a minimum percent in runs (100%), annotated, and subjected to a Student's t-test with comparing PROTAC-resistant cells vs. parental cells. Samples were run in biological triplicates (N=3). Parameters for the Student's t-test were the following: S0=2, two-sided using Permutation-based FDR<0.05. Volcano plots depicting differences in protein abundance were generated using R studio software and Prism graphics. Proteins repressed by MS934 or PD0325901 treatment (FDR<0.05) were imported into Metascape for pathway analysis.

### Example 2: MS934-Treatment Reduces CRAF, MEK1 and MEK2 Protein Levels in Cancer Cell Lines

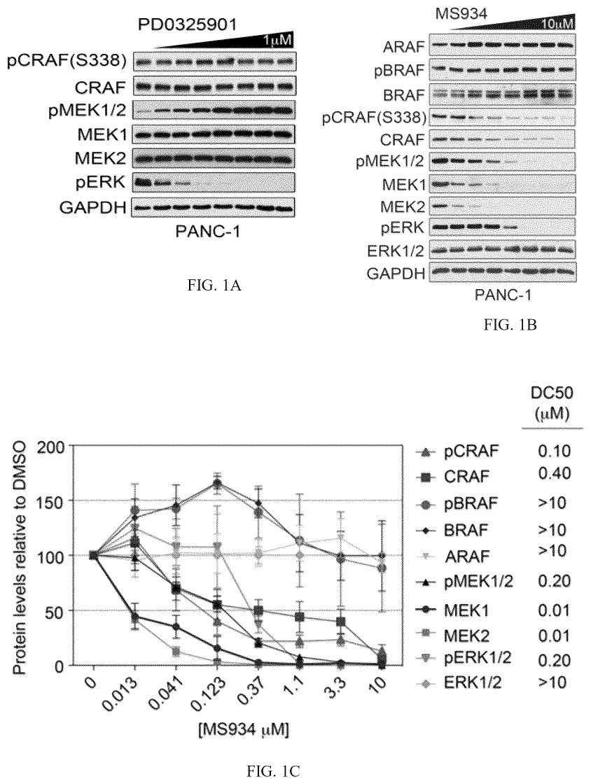

To explore the effect of MEK1 and MEK2 degradation on KRAS mutant cells, a KRAS mutant pancreatic cancer cell line, PANC-1, was treated with increasing concentrations of PD0325901 or MS934 for 24 hours and MEK1 and MEK2 protein levels were assessed, as well as other MAPK signaling components, by immunoblotting. A reduction in MEK1 and MEK2 protein levels was observed, as well as active phosphorylated MEK1/2(S217/S223) form with increasing doses of MS934 but not PD0325901 (see, FIGS. 1A and 1B). In contrast, elevated levels of p-MEK1/2(S217/S223) were observed with increasing doses of PD0325901, consistent with feedback-induced activation of CRAF following MEK-ERK pathway blockade. Surprisingly, a reduction in active phosphorylated CRAF(S338) and total CRAF protein levels in response to MS934 treatment in PANC-1 cells was observed that was not observed with PD0325901 (see, FIGS. 1A and 1). Quantitation of immunoblots revealed that the DC₅₀ (the concentration at which 50% of the target protein is degraded) of MS934 for MEK1 and MEK2 was 0.01 μM, while phosphorylated CRAF(S338) and MEK1/2(S217/S223) bands were reduced by half at 0.15 μM and 0.2 μM MS934, respectively, while the CRAF total proteins were reduced by 50% at a concentration of 0.4 μM MS934 (see, FIG. 1C). Notably, ARAF and BRAF protein levels were not reduced by MS934-treatment for 24 hours but rather showed an increase in protein levels at lower concentrations of MS934.

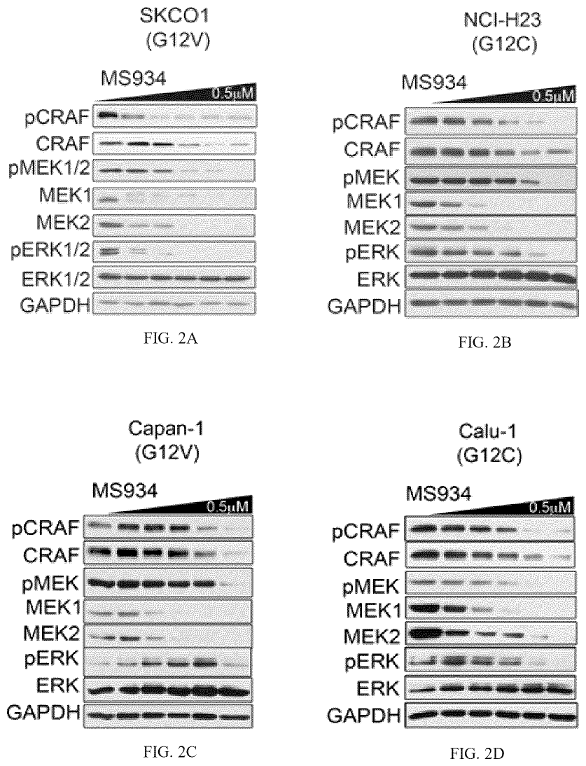

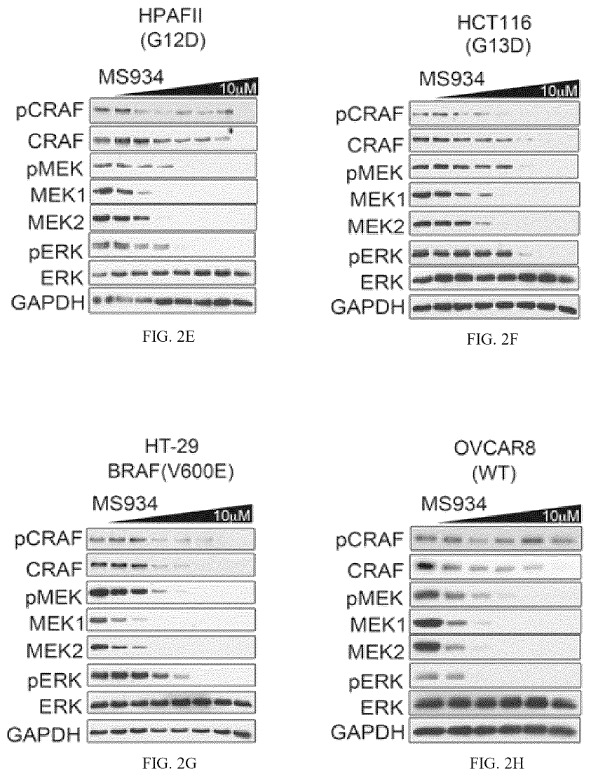

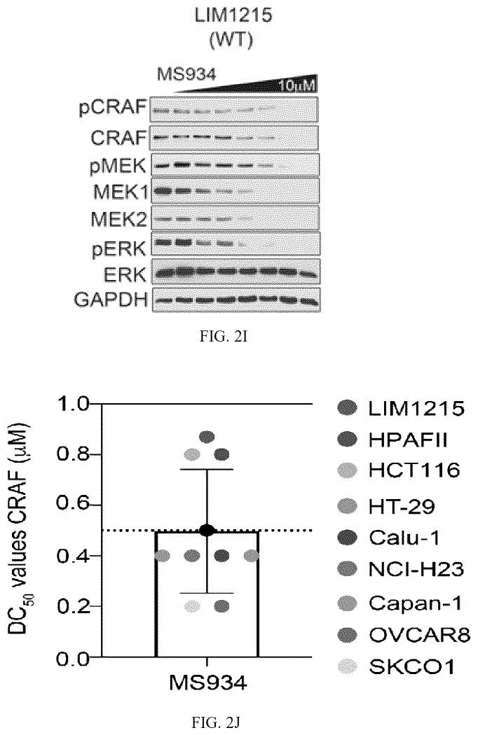

Next, studies explored whether MS934-treatment reduced CRAF and MEK1/2 protein levels in additional cancer cell lines including KRAS mutant cell lines (SKCO1, NCI-H23, Capan-1, Calu-1, HPAF-II, HCT-116, OVCAR8), a BRAF(V600E) mutant cell line (HT29), as well as a KRAS/BRAF wild-type cell line (LIM1215) (see, FIGS. 2A, 2B, 2C, 2D, 2E, 2F, 2G, 2H, and 2I). Notably, a dose dependent reduction in the active phosphorylated CRAF(S338) and MEK1/2(S217/S223) forms, as well as CRAF, MEK1 and MEK2 total protein levels was observed following MS934-treatment for 24 hours in all the cell lines tested. Quantitation of the concentration of MS934 required to reduce CRAF by half across the cancer cell lines ranged from 0.1 to 0.8 μM of MS934 with a median of 0.5 μM of MS934 (see, FIG. 2J). Together, these findings showed that MS934-treatment reduced CRAF protein levels in addition to the intended degrader-targets MEK1 and MEK2 in KRAS mutant, BRAFV600E and KRAS/BRAF-wild-type cancer cell lines.

### Example 3: MS934-Mediated CRAF Protein Degradation Occurs Via a PROTAC-Mechanism

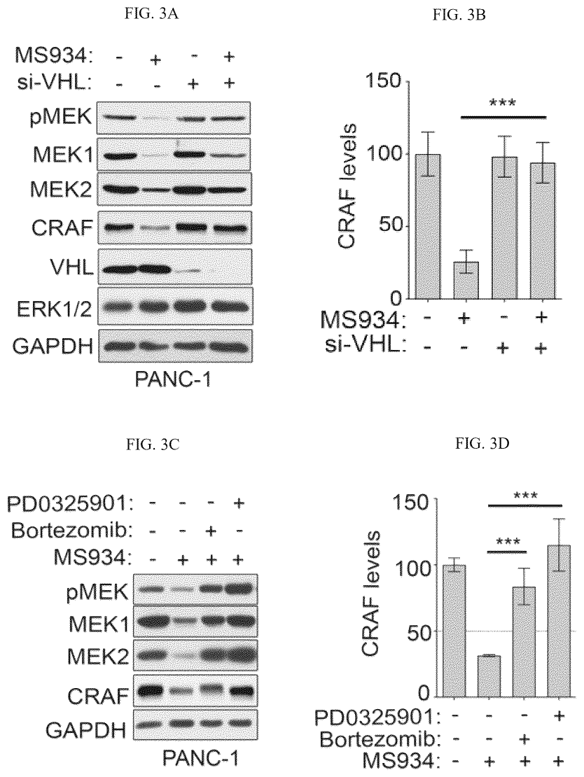

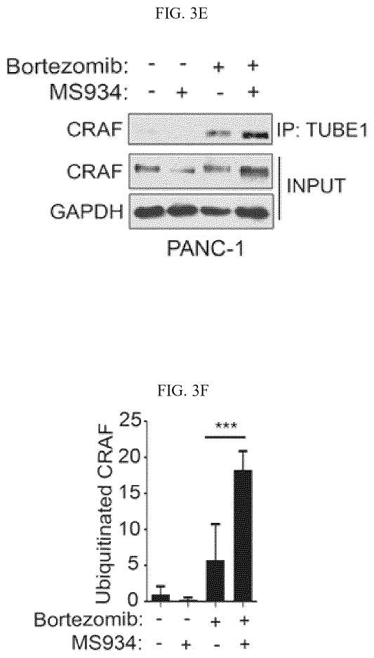

To determine if CRAF protein reduction by MS934-treatment was through a PROTAC-mechanism, VHL knockdown was performed, proteasome machinery was inhibited using bortezomib or cells were treated with excess PD0325901 in the presence or absence of MS934. Notably, siRNA-mediated knockdown of VHL rescued MEK1, MEK2 and CRAF protein levels, establishing that CRAF degradation required the VHL E3 ligase activity of MS934 (see, FIGS. 3A and 3B). Moreover, inhibiting the proteasome machinery in the presence of MS934 with the proteasome inhibitor, bortezomib, restored MEK1, MEK2 and CRAF protein levels (see, FIGS. 3C and 3D). In addition, treating cells with 100-fold excess of the warhead PD0325901 in the presence of MS934 outcompeted MS934-binding to targets, restoring MEK1, MEK2 and CRAF protein levels (see, FIGS. 3C and 3D). Furthermore, an increase in CRAF ubiquitination was observed in PANC-1 cells treated with MS934 and bortezomib, consistent with MS934-mediated degradation of CRAF occurring by a PROTAC mechanism (see, FIGS. 3E and 3F).

### Example 4: Relief of Negative Feedback Induces CRAF-MEK Interactions Causing Collateral Degradation of CRAF by MS934

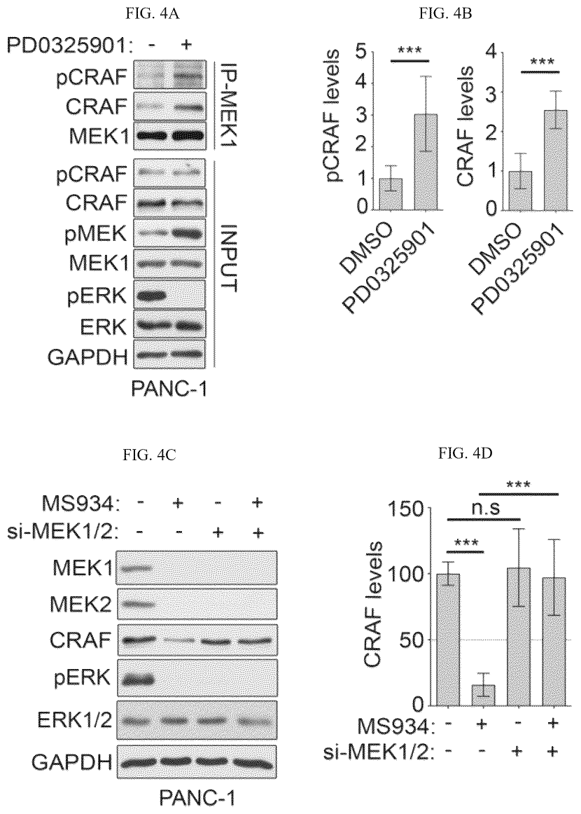

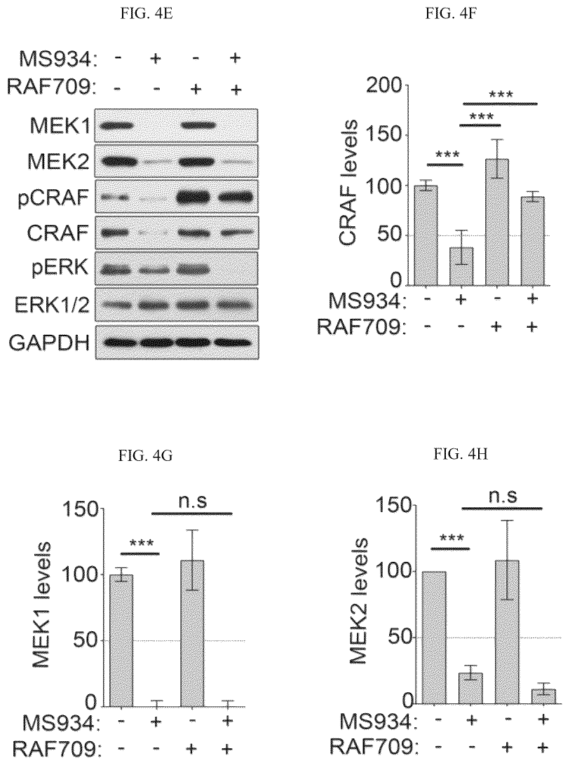

It has previously been shown that CRAF binds directly to MEK following PD0325901-treatment in KRAS mutant cells forming a protein complex due to relief of a negative feedback loop that occurs in cancer cells following inhibition of MEK-ERK activity (Lito et al., Cancer Cell, 2014, 25, 697-710). Consistent with these findings, this study showed that treatment of PANC-1 cells with the warhead of MS934, PD0325901, for 3 hours induced the CRAF-MEK1 interaction (see, FIGS. 4A and 4B). To test whether the interaction of CRAF and MEK1/2 was required for CRAF degradation by MS934, a knockdown of MEK1 and MEK2 in MS934-treated PANC-1 cells was performed. Importantly, the study showed that knockdown of MEK1 and MEK2 in PANC-1 cells does not cause reduced CRAF protein levels, signifying that loss of MEK1/2 alone does not result in concurrent loss of CRAF proteins (see, FIGS. 4C and 4D). Moreover, the study showed that the degradation of CRAF protein by MS934 required MEK1/2 proteins, whereby siRNA-mediated knockdown of MEK1/2 prevented the degradation of CRAF by MS934-treatment (see, FIGS. 4C and 4D). These findings also demonstrate that MS934 does not bind directly to CRAF. Finally, it was observed that inhibition of CRAF using the inhibitor, RAF709, restored CRAF protein levels but not MEK1 or MEK2 in MS934-treated PANC-1 cells, suggesting RAF709 prevents CRAF-MEK interactions blocking collateral degradation of CRAF by MS934 (see, FIGS. 4E, 4F, 4G, and 4H). Together, these findings establish that the collateral degradation of CRAF by MS934 requires the feedback induced CRAF-MEK complex formation that occurs following blockade of MEK-ERK activity.

### Example 5: MS934-Treatment Reduces Both CRAF Kinase-Dependent and Independent Signaling Providing Superior Growth Inhibition in KRAS Mutant Cells

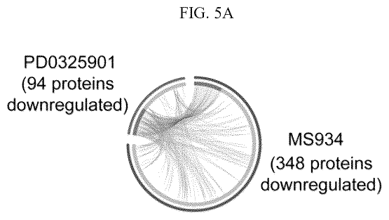

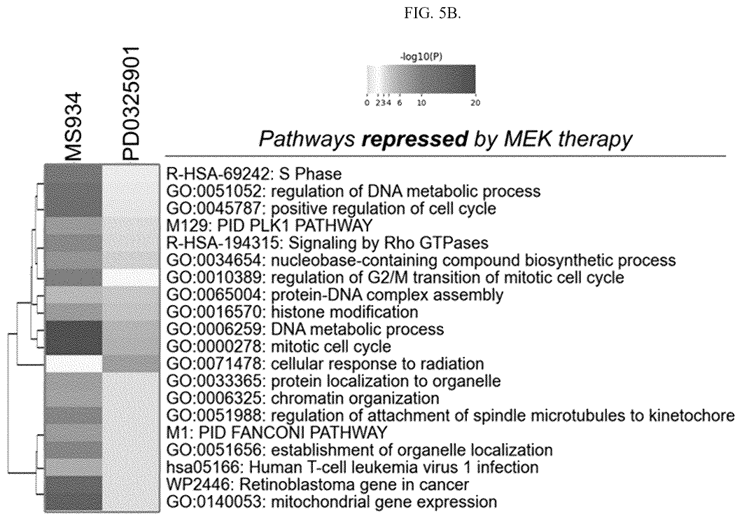

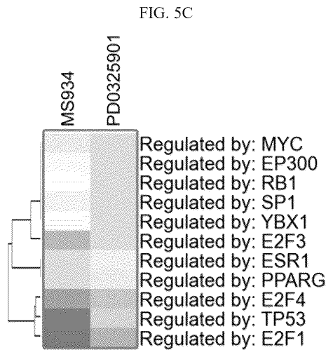

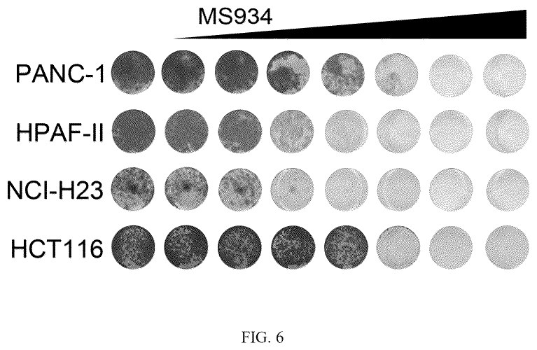

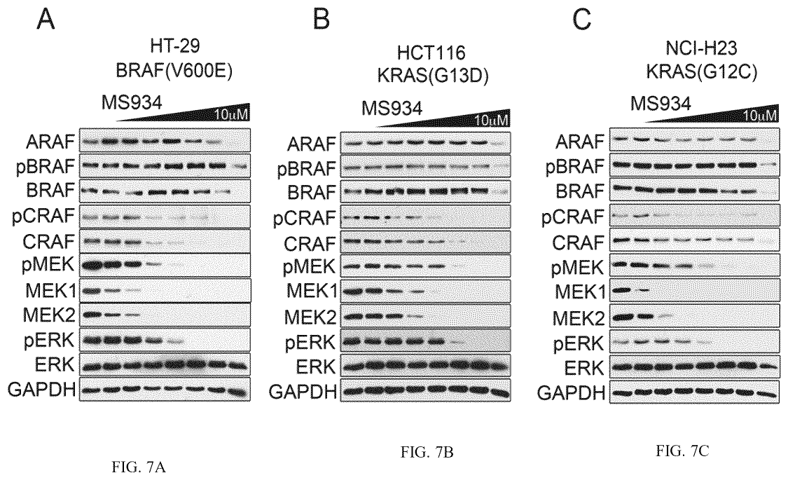

CRAF has been shown to have kinase-independent growth and survival functions outside the MEK-ERK pathway in KRAS mutant cells, making CRAF degradation an attractive therapeutic avenue (McCormick et al., Cancer Cell, 2018, 33, 158-159). To explore the difference between MEK inhibition and CRAF/MEK degradation on KRAS signaling, PANC-1 cells were treated with PD0325901 or MS934 and proteomics followed by pathway analysis were completed. The studies revealed that CRAF/MEK degradation reduced the expression of 348 proteins, while PD0325901-treatment reduced 94 proteins (see, FIG. 5A), demonstrating MS934-treatment exhibited a more pronounced effect on protein abundance than MEK inhibition. Metascape pathway analysis of proteins reduced by MEK inhibitor or CRAF/MEK degradation showed MS934-treatment exhibited a more potent reduction in the PLK1 pathway, signaling by Rho GTPases, and S phase signaling compared to PD0325901-treated PANC-1 cells (see, FIG. 5B). These findings are consistent with established kinase independent functions of CRAF, where prior studies have shown CRAF to signaling through PLK1, ROCK2 and CHEK2 signaling (McCormick et al., Cancer Cell, 2018, 33, 158-159). Moreover, MS934-treatment uniquely reduced pathways associated with chromatin organization, protein localization to organelle, regulation of attachment of spindle microtubules to kinetochore, Fanconi pathway, establishment of organelle localization, Human T-cell leukemia virus 1 infection, Retinoblastoma gene in cancer, and mitochondrial gene expression (see, FIG. 5B). Finally, transcription factors predicted to be reduced by treatments showed that MS934-treatment but not PD0325901-treatment uniquely reduced MYC signaling, an established driver in KRAS mutant pancreatic cancer (see, FIG. 5C). Treatment of KRAS mutant cell lines with MS934 reduced colony formation, with the majority of colony growth inhibited by 1 μM MS934-treatment (see, FIG. 6). Notably, PANC-1 cells have been previously shown to be intrinsically resistant to MEK inhibitor PD0325901-treatment (Zhong et al., PLoS One, 2013, 8, e77243), signifying MS934 causes growth inhibition of both MEK1-sensitive and MEK1-resistant KRAS mutant cell lines.

### Example 6: ARAF, BRAF and CRAF Protein Levels Reduced at Higher Concentrations of MS934

To determine if MS934-treatment altered levels of the RAF family (ARAF, BRAF and CRAF), cancer cell lines were treated with increasing doses of MS934. It was observed that treatment of cancer cell lines showed reduced levels of all three RAF members, ARAF, BRAF and CRAF at higher concentrations (10 μM). Notably, CRAF protein levels were reduced by about 50% at concentrations <1 μM, demonstrating that MS934 exhibits selectivity for degrading CRAF at concentrations <1 μM. Interestingly, a reduction in BRAF(V600E) protein levels was observed in HT-29 cells, suggesting MS934 could be beneficial in melanoma and colorectal cancers driven by activated BRAF by degrading both MEK1/2 and BRAF.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

### Example 7: Characterization of MEK1/2 Degraders Uncovers a Kinase-Independent Role for MEK1/2 in the Stabilization and Maturation of CRAF

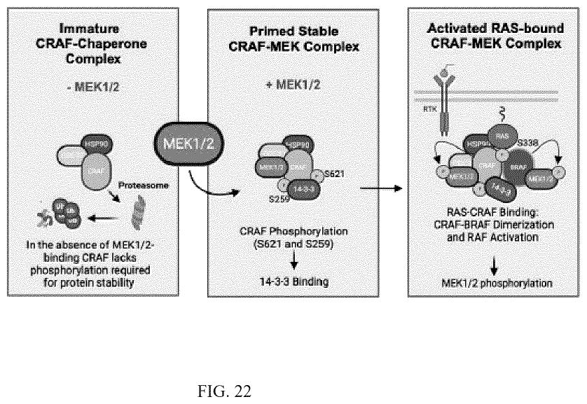

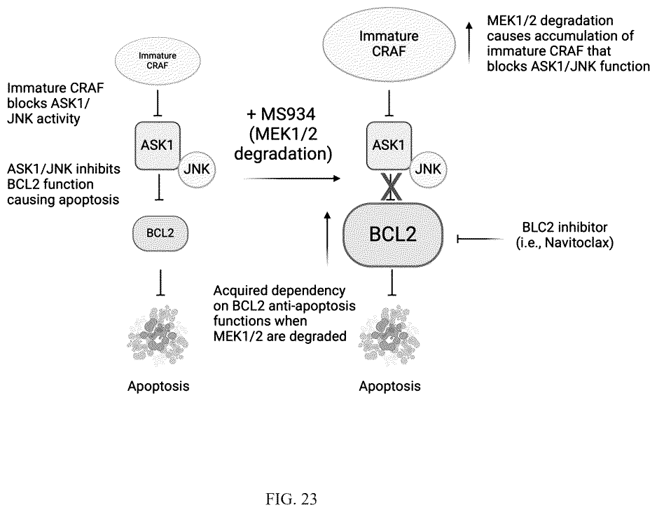

Altered MAPK signaling frequently occurs in human disease. MEK1 and MEK2 (MEK1/2) are central protein kinases in the MAPK signaling cascade that phosphorylate ERK1/2 promoting cell growth. MEK1/2 degraders offer a strategy to characterize both kinase-dependent and independent functions of MEK1/2. As described herein, MEK1/2 degradation, but not kinase inhibition, caused the subsequent degradation of upstream kinase CRAF via a cell-intrinsic mechanism. MEK1/2 binding to CRAF, but not MEK1/2 catalytic activity, was important for CRAF protein stability and maturation to a functional kinase. In the absence of MEK1/2, a minor pool of newly synthesized immature CRAF that had anti-apoptotic functions remained. Also described herein is a stable primed CRAF-MEK1/2 signaling complex existed in cells that required RAS binding to potentiate MEK-ERK phosphorylation. Together, the results described herein demonstrate a kinase-independent function of MEK1/2, while contextualizing MEK1/2 as an integral component of the CRAF activation cycle beyond the conventional CRAF-MEK kinase-substrate paradigm (see, FIG. 22).

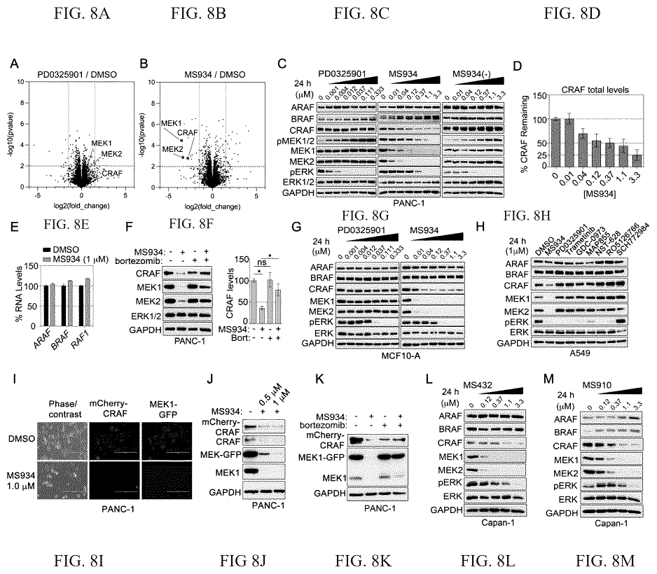

MEK1/2 Degradation but not Inhibition Caused Loss of Upstream Kinase CRAF. Global proteomics was performed on PANC-1 cells treated with equal concentrations of the MEK1/2 inhibitor, PD0325901 or the PD0325901-based MEK1/2 degrader, MS934 (Hu, J., et al. 2020. J Med Chem, 63, 15883-15905). Strikingly, in addition to MEK1/2 protein loss following 24-hour MS934 treatment, a significant reduction in total CRAF protein levels was observed that was not detected in PD0325901-treated cells (FIGS. 8A-B, 15A). Similarly, immunoblot analysis showed a dose-dependent reduction in total CRAF protein levels concomitant with MEK1/2 degradation in response to MS934 treatment of PANC-1 cells that was not observed with PD0325901 or a negative control PROTAC (MS934[−]) unable to recruit VHL (FIG. 8C). Quantitation of immunoblots revealed that CRAF total protein levels were reduced to ~25% of the control by the 3.3 mM dose (FIG. 8D). Notably, ARAF and BRAF protein levels were not reduced by 24 hours MS934 treatment, with BRAF showing a modest increase in protein levels (FIG. 8D). No change in RAF1, ARAF or BRAF mRNA levels in PANC-1 cells was observed following 1 mM MS934 treatment, signifying that the reduction in CRAF protein level by MS934 occurred at the post-transcriptional level (FIG. 8E). Co-treatment of PANC-1 cells with MS934 and the proteasome inhibitor bortezomib restored MEK1, MEK2 and CRAF protein levels, demonstrating the involvement of the proteasome in the downregulation of CRAF by MS934 (FIG. 8F).

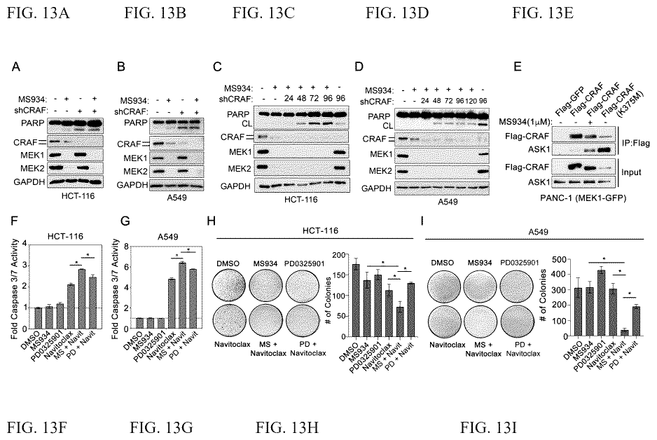

To determine whether loss of CRAF by MS934 treatment occurred in other cell lines, a panel of cancer cell lines harboring KRAS mutations (FIG. 15B), NF1-loss (FIG. 15C), BRAF mutation (FIG. 15D), as well as non-tumorigenic cells (FIGS. 8G, 15E) was tgreasted with escalating doses of MS934. A dose-dependent reduction in CRAF total protein levels but not ARAF, or BRAF was observed with MS934 treatment but not PD0352901 in non-tumorigenic cell line MCF10-A (FIG. 8G), and MS934 reduced CRAF levels across the cell lines tested (FIGS. 15B-E). Additionally, treatment of cells with other MEK1/2 inhibitors or an ERK1/2 inhibitor failed to reduce CRAF like MS934, demonstrating that loss of CRAF was specific to MEK1/2 degradation (FIG. 8H). Like PANC-1 cells, CRAF loss following MS934 treatment could be restored via blocking the proteasome in A549, and no change in RAF1 mRNA levels was observed with escalating doses of MS934 in HCT-116 cells (FIGS. 15F-G).

Next, mCherry-CRAF or MYC-CRAF was exogenously expressed in PANC-1 cells co-expressing MEK1-GFP, treated with MS934 for 24 hours, and measured CRAF protein levels by fluorescence imaging or immunoblotting. Notably, MS934 treatment reduced exogenous mCherry-CRAF and MEK1-GFP in PANC1 cells (FIGS. 8I-J). Similarly, MS934 treatment reduced exogenous MYC-CRAF levels in a dose-dependent manner in PANC1 co-expressing MEK1-GFP (FIG. 15H). Blocking the proteasome using bortezomib in combination with MS934 restored mCherry-CRAF levels (FIG. 8K).

To determine whether other MEK1/2-targeting PROTACs caused downregulation of CRAF similar to MS934, Capan-1 cells was treated with the VHL-based PD0325901-PROTAC MS432 (Wei, J., et al. 2019. Journal of Medicinal Chemistry, 62, 10897-10911), or MS910 (Hu, J., et al. 2020. J Med Chem, 63, 15883-15905), which uses a CRBN-recruiting moiety. Notably, treatment of Capan-1 cells with escalating doses of MS432 (FIG. 8L), or MS910 (FIG. 8M) reduced MEK1/2 and CRAF protein levels, demonstrating that using either VHL or CRBN-recruiting moieties decreased both CRAF and MEK1/2 protein levels in cells. Thus, these data show that MEK1/2 have kinase-independent functions, intrinsic to the cell types tested, beyond their described kinase-dependent function in activating the MAPK signaling cascade through ERK phosphorylation.

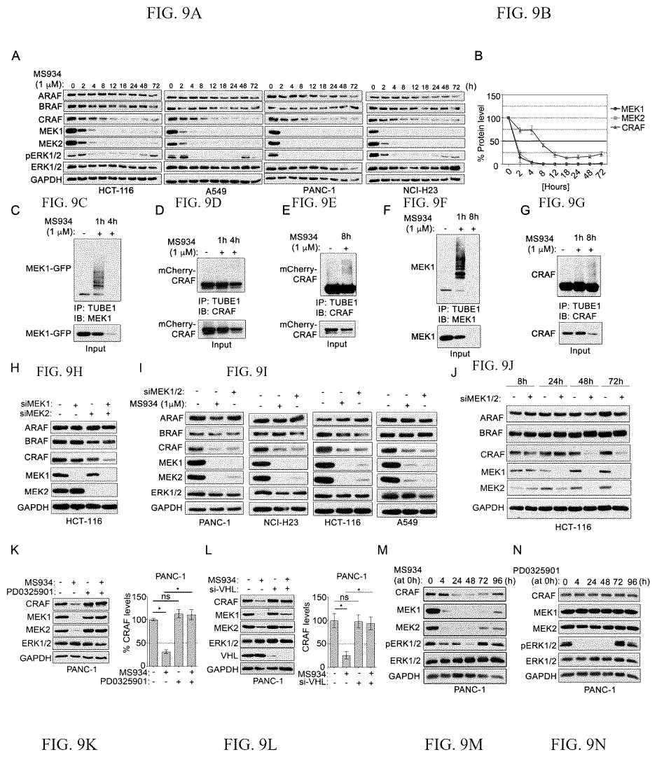

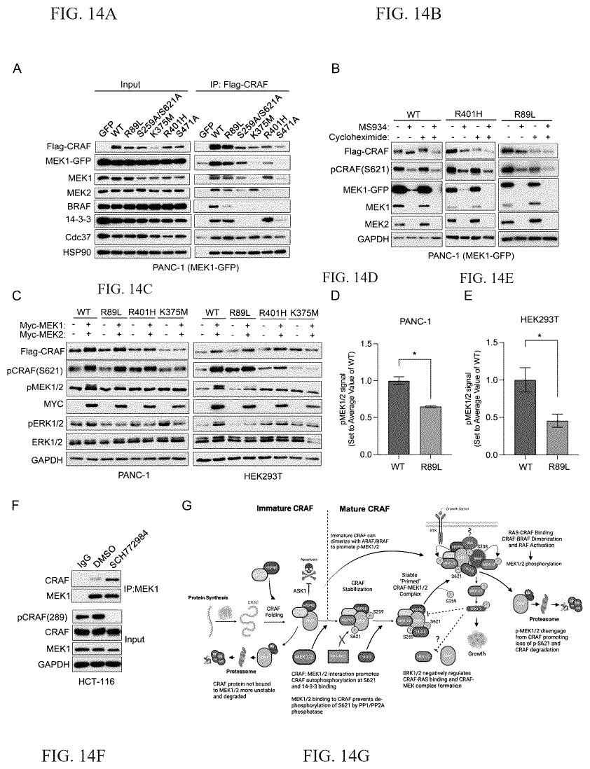

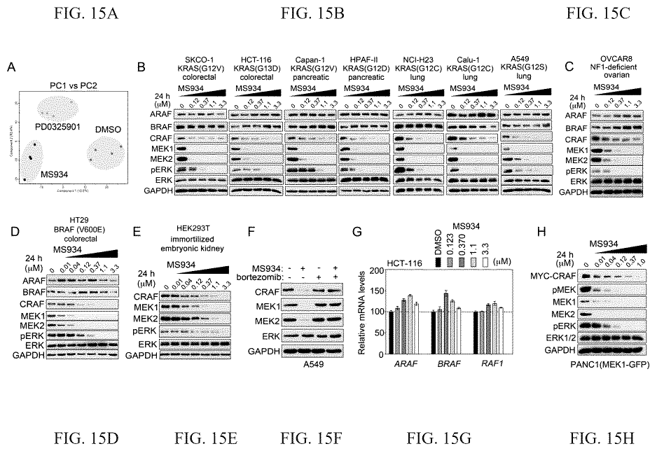

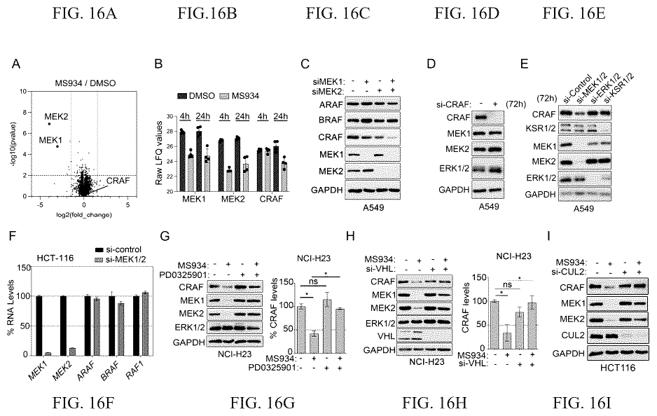

MEK1/2 are Important for Preserving CRAF Protein Stability. To explore the timing of CRAF protein loss in relation to MEK1/2 degradation by MS934, PANC1, NCI-H23, HCT-116, and A549 cell lines were treated with 1 mM MS934 for 2, 4, 8, 12, 18, 24, 48, or 72 hours and performed immunoblotting. Degradation of MEK1 and MEK2 proteins occurred within the first 2 hours of MS934 treatment, however, CRAF protein levels were not reduced by 50% until 8 hours (FIGS. 9A-B). Similarly, global proteomics showed that MEK1/2 but not CRAF protein were depleted following 4-hour MS934 treatment (FIG. 16A), requiring 24 hours of MS934 treatment to observe CRAF loss (FIG. 16B). Notably, minimal changes in ARAF or BRAF protein levels were observed across the 72-hour time course (FIG. 9A).

To further characterize the timing and process of CRAF degradation following MS934 treatment, ubiquitination of exogenous MEK1-GFP and mCherry-CRAF in PANC-1 cells in response to MS934 was measured using TUBE immunoprecipitations (TUBE-IP), a method for enriching proteins containing poly-ubiquitination (Hjerpe, R., et al. 2009. EMBO reports, 10, 1250-1258-1258). Analysis of MS934-mediated ubiquitination using TUBE1-IP, showed that MEK1-GFP was ubiquitinylated at 1 hour after 1 mM MS934 treatment, while CRAF showed no evidence of ubiquitination at 1 or 4 hours post-MS934 treatment (FIGS. 9C-D). However, consistent with immunoblot analysis, TUBE1-IP at the later 8-hour time point showed ubiquitination of mCherry-CRAF following 1 mM MS934 treatment (FIG. 9E). Similarly, TUBE-IP of endogenous MEK1 or CRAF from HCT-116 cells following 1 mM MS934 treatment showed that ubiquitination of endogenous MEK1 but not CRAF was observed at 1 hour post-MS934 treatment, with CRAF showing ubiquitination at 8 hours post-MS934 treatment (FIGS. 9F-G).

Next, genetic knockdown of MEK1, MEK2 or both using siRNAs in HCT116 cells was performed. Genetic depletion of MEK1 or MEK2 alone did not reduce CRAF protein levels, whereas the simultaneous knockdown of both MEK1/2 reduced CRAF levels, establishing that both MEK1 and MEK2 were important for maintaining CRAF protein levels (FIGS. 9H, 16C). Whereas knockdown of MEK1/2 reduced CRAF, as observed with MS934 treatment, ARAF or BRAF protein levels were unaffected by MEK1/2 knockdown in PANC-1, NCI-H23, HCT-116 and A549 cells (FIG. 9I). MEK1/2 knockdown over a 72-hour period in HCT116 cells revealed that MEK1/2 proteins were reduced at 24 hours, while CRAF total protein levels remained until 48 hours, demonstrating MEK1/2 reduction preceded CRAF loss following MEK1/2 knockdown (FIG. 9J). Notably, knockdown of CRAF did not reduce MEK1/2 protein levels (FIG. 16D), and genetic depletion of the KSR1/2 scaffold proteins or ERK1/2 also failed to decrease CRAF protein levels as was observed with MEK1/2 knockdown (FIG. 16E). No changes in mRNA levels of RAF1 were detected following MEK1/2 knockdown (FIG. 16F).

Next, it was evaluated whether preventing MS934-mediated degradation of MEK1/2 would rescue CRAF protein levels using excess PD0325901 or performing knockdown of E3 ligase components VHL or CUL2 required by MS934. Restoration of MEK1/2 proteins in PANC-1 or NCI-H23 cells was observed by combining MS934 with excess PD0325901 treatment (FIGS. 9K, 16G). Similarly, preventing PROTAC-mediated degradation via VHL knockdown (FIGS. 9L, 16H) blocked CRAF degradation, restoring CRAF protein levels. Additionally, knockdown of CUL2 in MS934-treated HCT-116 cells restored MEK1/2 and CRAF protein levels (FIG. 16I). Finally, analysis of CRAF and MEK1/2 protein levels in PANC-1 cells following a single treatment of MS934 over a 96-hour time course, showed that CRAF protein levels returned to control levels by 96 hours coinciding with the partial return of MEK1 and MEK2 (FIG. 9M). No changes in CRAF or MEK1/2 protein levels were detected over 96 hours following PD0325901 treatment (FIG. 9N). Taken together, these data demonstrate that degradation of MEK1/2 promoted the subsequent degradation of CRAF in KRAS mutant cells several hours after MS934 treatment or MEK1/2 genetic knockdown by a proteosome-dependent process, showing that CRAF protein stability was dependent on the presence of MEK1/2 proteins.

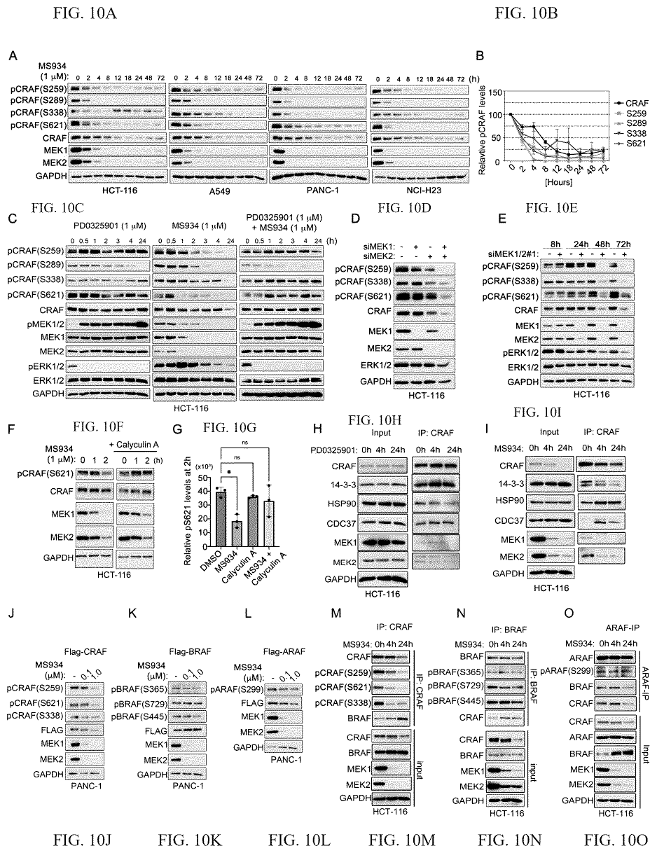

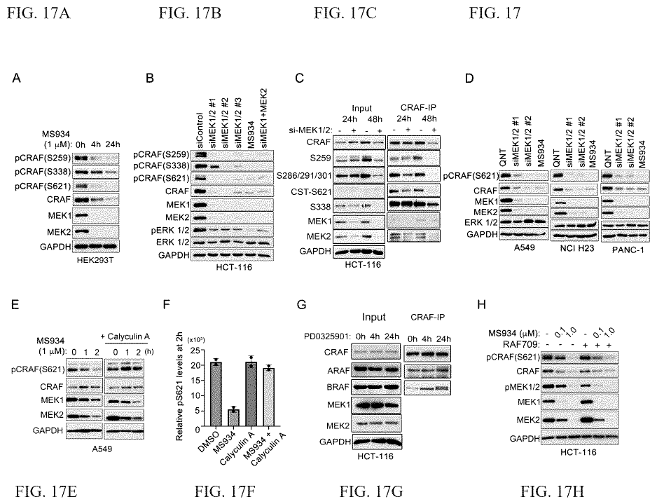

MEK1/2 Degradation Caused Rapid Dephosphorylation of CRAF at S621 Promoting Misfolding and Degradation. CRAF activation is regulated by several phosphorylation events (Dhillon, A. S., et al. 2007. Cell Cycle, 6, 3-7; Noble, C., et al. 2008. Mol Cell, 31, 862-72; Reiterer, G., et al. 2010. Cell Cycle, 9, 3297-304; Yu, A., et al. 2023. J Biol Chem, 299, 105188; Xiang, X., et al. 2002. J Biol Chem, 277, 44996-5003; and Zang, M., et al. 2008. J Biol Chem, 283, 31429-37). To explore the effect of MEK1/2 degradation on CRAF phosphorylation, immunoblot analysis of HCT-116 cells treated with 1 mM MS934 across a 72-hour time course was performed (FIGS. 10A-B). Reduction of p-S289/296/301 was observed within 2 hours of MS934 treatment, consistent with rapid inhibition of ERK1/2 signaling (Dougherty, M. K., et al. 2005. Molecular Cell, 17, 215-224). Whereas CRAF p-S338 levels varied in HCT-116 cells over time, with levels being reduced at later time points, decreased p-S338 levels were observed within 4 hours of MS934 treatment in A549, PANC-1, and NCI-H23 cell lines, signifying inhibition of CRAF activity (King, A. J., et al. 1998. Nature, 396, 180-3). CRAF p-S259 was downregulated within 2 hours post MS934 in the cell lines tested. Autophosphorylation of S621 by CRAF is important for protein stability, where mutation of S621 to alanine promotes CRAF degradation (Noble, C., et al. 2008. Mol Cell, 31, 862-72). Notably, decreased levels of p-S621 were observed 2 hours post-MS934 treatment in the cells, preceding CRAF protein degradation. Reduced CRAF p-S621 was also observed in KRAS wild-type HEK293T cells in response to 4-hour MS934 treatment (FIG. 17A).

To determine if blocking MEK1/2 degradation using excess PD0325901 could rescue the dephosphorylation of CRAF post-MS934 treatment, HCT-116 cells were treated with 1 mM PD0325901, MS934 or the combination and monitored CRAF phosphorylation across time (FIG. 10C). Reductions in CRAF phosphorylation at S259, S338 and S621 were observed within 2 hours of MS934 treatment but not in response to PD0325901-treatment, whereas decreased phosphorylation of ERK1/2 negative feedback sites S289/296/301 occurred 2 hours post-PD0235901 or MS934 treatment. Notably, combined treatment of MS934 and excess PD0325901 rescued the MS934-mediated dephosphorylation of CRAF at S259, S338, and 5621, but not at the ERK1/2-regulated sites 5289/296/301.

Next, the effect of knockdown of MEK1, MEK2, or both on CRAF phosphorylation was examined. Genetic depletion of MEK1 or MEK2 alone did not reduce CRAF phosphorylation or total protein levels in HCT-116 cells, whereas the simultaneous knockdown of both MEK1/2 with 3 distinct siRNAs (MEK1/2-siRNA #1, MEK1/2-siRNA #2 or MEK1/2-siRNA #3) that targeted shared regions within MEK1 and MEK2 reduced CRAF phosphorylation at S259, S338 and 5621 (FIG. 10D) as did combining MEK1 and MEK2 siRNAs (FIG. 17B). Additionally, immunoprecipitation of endogenous CRAF from HCT-116 cells treated with MEK1/2 siRNAs for 24 or 48 hours showed that MEK1/2 knockdown reduced CRAF phosphorylation at S259, S338, and 5621 by 48 hours (FIG. 17C). Decreased phosphorylation of CRAF at 5621, as well as total CRAF protein levels were also observed in A549, PANC1, and NCI-H23 cell lines following MEK1/2 knockdown (FIG. 17D). MEK1/2 knockdown over a 72-hour period in HCT-116 cells revealed that MEK1/2 proteins were reduced at 24 hours, whereas CRAF phosphorylation at S259, S338, and 5621 was not reduced until 48 hours (FIG. 10E).

Phosphatases, including PP1/PP2, dephosphorylate CRAF and other components of the MAPK signaling cascade (Boned del Rio, I., et al. 2019. Proc Natl Acad Sci USA, 116, 13330-1333; and Ory, S., et al. 2003. Curr Biol, 13, 1356-64). To investigate whether PP1/PP2 phosphatases contributed to the dephosphorylation of CRAF by MS934, HCT-116 or A549 cells were co-treated with MS934 and the PP1/PP2A inhibitor Calyculin A for 1 or 2 hours and measured CRAF phosphorylation at 5621 by immunoblot. Notably, co-treatment of MS934 and Calyculin A rescued the MS934-mediated dephosphorylation of 5621 at the 2-hour time point in HCT-116 (FIGS. 10F-G) and A549 cells (FIGS. 17E-F), signifying that PP1/PP2 were involved in the rapid dephosphorylation of CRAF at S621 that occurred following MEK1/2 degradation.

Proper protein folding, stability, and activation of CRAF are regulated by interacting proteins 14-3-3, HSP90, and CDC37 (Thorson, J. A., et al. 1998. Mol Cell Biol, 18, 5229-38; Schulte, T. W., et al. 1995. J Biol Chem, 270, 24585-8; Mitra, S., et al. 2016. J Biol Chem, 291, 24579-24593; and Garcia-Alonso, S., et al. 2022. Mol Cell, 82, 3438-3452 e8). Binding of 14-3-3 to CRAF phosphorylated at S259 and 5621 is important for the regulation of CRAF kinase activity (Thorson, J. A., et al. 1998. Mol Cell Biol, 18, 5229-38). To explore the effect of MEK1/2 degradation or inhibition on the interaction of CRAF with 14-3-3, CDC37, or HSP90, immunoprecipitations of endogenous CRAF from HCT-116 cells treated with 1 mM PD0325901 or MS934 for 4 or 24 hours was performed. In vehicle-treated HCT-116 cells, a protein complex of CRAF, HSP90, CDC37, 14-3-3, and MEK1/2 was observed (FIGS. 10H-I). PD0325901 did not alter the binding of MEK1/2 to CRAF, whereas immunoprecipitation of CRAF from MS934-treated cells showed reduced MEK1/2 bound to CRAF (FIGS. 10H-I). A reduction in 14-3-3 binding to CRAF in response to MS934 but not PD0325901-treatment at 4 and 24 hours was observed, consistent with rapid dephosphorylation of CRAF at S259 and S621, both docking sites for 14-3-3 (FIGS. 10H-I). Conversely, an increase in CDC37 and HSP90 interaction was observed following 4 and 24 hours of treatment with MS934 but not PD0325901 (FIGS. 10H-I), showing that dephosphorylation of S621 caused CRAF misfolding, promoting increased chaperone activity by HSP90 and CDC37 (Noble, C., et al. 2008. Mol Cell, 31, 862-72; Silverstein, A. M., et al. 1998. J Biol Chem, 273, 20090-5; and Grammatikakis, N., et al. 1999. Mol Cell Biol, 19, 1661-72). Thus, although the possible importance of CRAF dephosphorylation at other sites (i.e. p-S259 and p-S338) cannot be excluded, taken together these data demonstrate that dephosphorylation of CRAF at S621 contributed to the observed decreased stability and increased degradation of CRAF that occurred in response to MEK1/2 protein loss.

MEK1/2 degradation caused loss of CRAF but not ARAF or BRAF proteins. Next, Flag-tagged ARAF, BRAF, or CRAF was expressed in PANC-1 cells co-expressing MEK1-GFP, treated with MS934 for 24 hours and monitored phosphorylation. MS934 treatment reduced Flag-CRAF phosphorylation and total protein levels (FIG. 10J), but no change in phosphorylation of Flag-BRAF at S362, S446, or S729 (equivalent sites to S259, S338 and S621 in CRAF) was observed (FIG. 10K). MS934 treatment marginally increased Flag-BRAF total protein levels. Minimal reduction in phosphorylation of Flag-ARAF at S299 (equivalent site to S338 in CRAF) and Flag-ARAF protein levels was observed following MS934 treatment (FIG. 10L). Similarly, immunoprecipitation of endogenous CRAF from MS934-treated HCT-116 cells showed reduced phosphorylation and total protein levels (FIG. 10M), whereas immunoprecipitation of BRAF (FIG. 10N) or ARAF (FIG. 10O) showed no reductions in phosphorylation or total protein levels at 4 or 24 hours post-MS934 treatment. MEK inhibition promotes CRAF-BRAF dimerization and activation (Lamba, S., et al. 2014. Cell Reports, 8, 1475-1483. Despite reduced CRAF levels, a robust increase in BRAF interaction with CRAF was observed in response to MS934 treatment like PD0325901 (FIGS. 10M-N, 17G). Combining RAF709 with MS934 inhibited p-MEK1/2 levels more than single agents, suggesting BRAF activation occurred following MEK1/2 degradation (FIG. 17H). A minor reduction in ARAF binding to CRAF or BRAF was observed 24 hours post-MS934 treatment (FIG. 10O). Thus, MEK1/2 degradation differentially affects RAFs, causing CRAF degradation and BRAF activation.

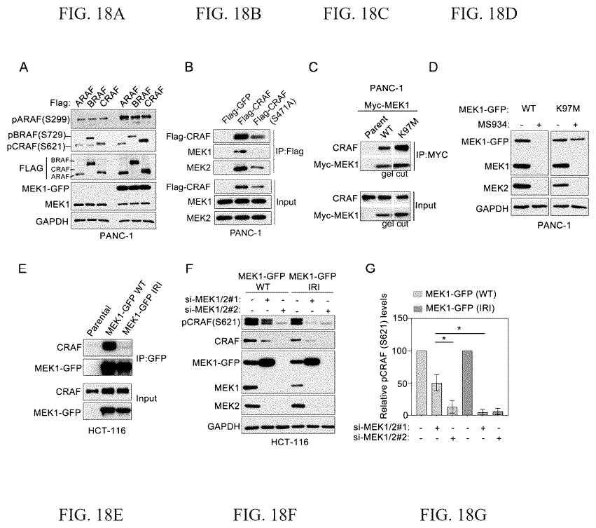

MEK1/2 Binding to CRAF but not Kinase Activity Required for CRAF Phosphorylation at S621 and Protein Stability. The knockdown and degradation studies showed that MEK1/2 was important for CRAF stability and activation, therefore, it was tested whether increasing MEK1/2 protein levels would also promote CRAF stabilization and activation. Moreover, it has been shown that co-expression of MEK1 and CRAF promoted increased phosphorylation of MEK1/2 and ERK1/2 (Leicht, D. T., et al. 2013. Biochimica et Biophysica Acta (BBA)—Molecular Cell Research, 1833, 976-986). The results described herein show that increasing concentrations of Myc-MEK1 and/or Myc-MEK2 caused stabilization of Flag-CRAF total protein levels, as well as elevated levels of p-S621, and p-MEK1/2 (FIG. 11A). In contrast, Flag-BRAF protein levels were equivalent in the presence or absence of MEK1-GFP expression, as was phosphorylation of BRAF at S729 (FIG. 18A). Flag-ARAF showed some increase in phosphorylation at S299 and total protein expression when co-expressed with MEK1-GFP (FIG. 18A), demonstrating that overexpression of MEK1/2 can also alter ARAF protein levels.

Phosphorylation of CRAF at S471 is important for MEK1-binding, whereby S471A mutation impairs MEK1 binding (Zhu, J., et al. 2005. Mol Biol Cell, 16, 4733-44). Immunoprecipitation of Flag-CRAF(S471A) from PANC-1 cells exhibited impaired MEK1 binding compared to Flag-CRAF wild-type whereas MEK2 is largely unaffected (FIG. 18B). Co-expression of Flag-CRAF(S471A) and MYC-MEK1/2 did not increase Flag-CRAF(5471) total protein or p-S621 levels as was observed for Flag-CRAF wild-type (FIGS. 11B-C). Together, these data show that reducing MEK1-binding to CRAF impaired CRAF phosphorylation and protein stability.

To determine if MEK1/2 catalytic activity was important for CRAF stability, the conserved lysine in MEK1 (K97) or MEK2 (K101) was mutated rendering MEK1/2 kinase-dead (Pearson, G., et al. 2000. J Biol Chem, 275, 37303-6). The MEK1(K97M) mutant maintained its ability to interact with CRAF (FIG. 18C). Co-expression of Flag-CRAF and Myc-MEK1(K97M)/Myc-MEK2(K101M) increased p-S621, Flag-CRAF total protein and p-MEK1/2 like co-expression of wild-type Myc-MEK1/2 (FIG. 11D). The K97M and K101M MEK1/2 mutants are resistant to PD0325901 (Hatzivassiliou, G., et al. 2013. Nature, 501, 232-6 and MS934 (FIG. 18D). Dose escalation of Myc-MEK1(K97M) or Myc-MEK2(K101M) in MS934-treated cells increased Flag-CRAF, p-S621 and p-MEK1/2 levels in the absence of endogenous MEK1/2 (FIGS. 11E-F). Thus, catalytic activity of MEK1 or MEK2 was not required for promoting p-S621, protein stabilization, and activation of CRAF. To further explore the role of MEK1 binding in regulating CRAF stability, alanine substitutions were made for Met 308 and Ile 310 in MEK1 which impairs binding to CRAF (IRI) (Mckay, M. M., et al. 2009. Proc Natl Acad Sci USA, 106, 11022-7). Immunoprecipitation of MEK1-GFP(IRI) from cells showed reduced binding to CRAF (FIG. 18E). Next, MEK1/2 (#1) siRNA-resistant MEK1-GFP(IRI) mutants were generated and the ability of the RAF binding mutant to restore endogenous CRAF p-S621 or total protein levels in HCT-116 and PANC-1 cells were evaluated. Expression of siRNA-resistant MEK1-GFP(WT) partially rescued p-S621 and CRAF levels that occurred post MEK1/2 knockdown (FIGS. 11G-H, 18F-G). Notably, expression of MEK1/2 siRNA (#1)-resistant MEK1-GFP(IRI) did not restore CRAF p-S621 or total protein levels to the extent of MEK1-GFP(WT) expression in PANC-1 or HCT-116 cells, further establishing that binding of MEK1 to CRAF was important for promoting CRAF p-S621 and protein stability.

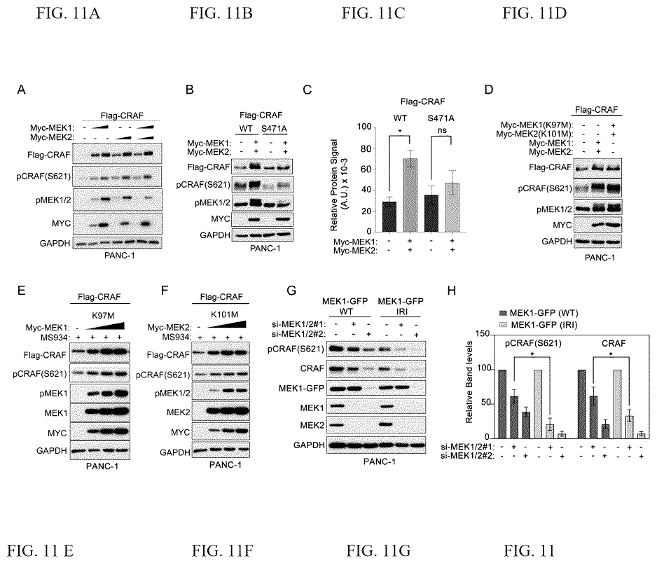

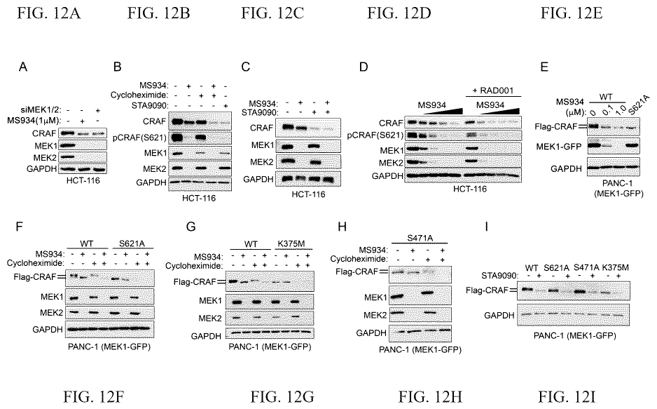

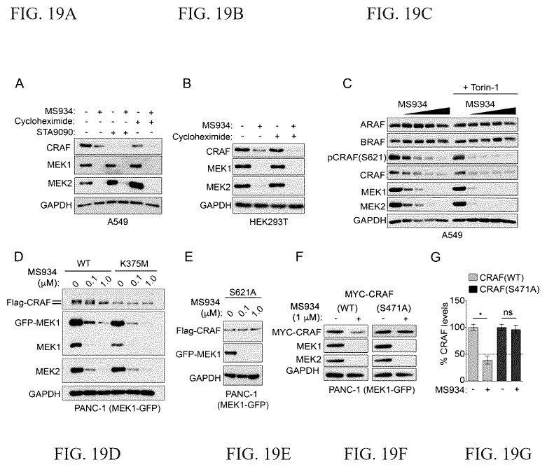

MEK1/2 Binding to CRAF Important for Maturation into Functional Kinase. Distinct cellular pools of CRAF exist in cells that include 1) newly synthesized immature CRAF and 2) mature active form of CRAF phosphorylated at S621 (Mitra, S., et al. 2016. J Biol Chem, 291, 24579-24593). HSP90 inhibitors selectively degrade immature newly synthesized CRAF, preventing the formation of the active mature CRAF containing p-S621 (Mitra, S., et al. 2016. J Biol Chem, 291, 24579-24593). In the characterization of MEK1/2 degradation or knockdown, it was repeatedly observed that a minor pool (~25%) of CRAF remained post-MS934 treatment (FIGS. 8C, 12A) or MEK1/2 knockdown (FIG. 12A). To characterize the observed residual CRAF, HCT-116 cells were treated with MS934, the protein synthesis inhibitor, cycloheximide, the HSP90 inhibitor STA9090, or the combinations of MS934 and cycloheximide or STA9090. Notably, combining MS934 with cycloheximide enhanced the degradation of CRAF compared to single agents (FIG. 12B). HSP90 inhibition reduced CRAF levels like combining MS934 and cycloheximide, while combining MS934 and STA9090 did not reduce CRAF greater than HSP90 inhibition alone (FIG. 12C). Cycloheximide treatment also enhanced degradation of CRAF in response to MS934 treatment in A549 and HEK293T (FIG. 19A-B). Additionally, combined blockade of translation using MTOR inhibitor RAD001 in MS934-treated HCT-116 cells (FIG. 12D) or Torin-1 in MS934-treated A549 cells (FIG. 19C) enhanced the depletion of p-S621 and total CRAF levels. Together, these findings show that the minor pool of CRAF remaining post-MEK1/2 degradation was newly synthesized immature CRAF that was sensitive to protein synthesis or MTOR inhibition.

Exogenous expression of CRAF presents as two bands in immunoblots, the upper band being the phosphorylated form of CRAF that contained p-S621 amongst other phosphosites (Ferrier, A. F., et al. 1997. J Biol Chem, 272, 2136-42). Notably, MS934 treatment reduced the upper Flag-CRAF band, while some residual lower Flag-CRAF band remained that migrated like the immature Flag-CRAF(S621A) mutant (FIG. 12E). To explore the stability of mature and immature pools of CRAF, PANC-1(MEK1-GFP) cells co-expressing maturation-competent Flag-CRAF(WT), or maturation-noncompetent Flag-CRAF(S621A), or kinase-dead Flag-CRAF(K375M) mutant were treated with 1 mM MS934, cycloheximide, or the combination. MS934 treatment reduced the upper and partially the lower Flag-CRAF(WT) band, while cycloheximide reduced the lower Flag-CRAF(WT) band, sparing the upper phosphorylated band (FIGS. 12F-G). Notably, the combination of MS934 and cycloheximide reduced both Flag-CRAF(WT) upper and lower bands to a greater extent than single agents, confirming that the lower band observed post-MS934 was newly synthesized Flag-CRAF (FIGS. 12F-G). Additionally, whereas MS934 treatment reduced the upper mature FLAG-CRAF(WT) band, it did not reduce immature Flag-CRAF(S621A) or Flag-CRAF(K375M) levels (FIG. 19D-E). In contrast, cycloheximide treatment depleted the majority of Flag-CRAF(S621A) or Flag-CRAF(K375M) levels, and the combination of MS934 and cycloheximide was minimally better than cycloheximide treatment alone (FIGS. 12F-G). Expression of the Flag-CRAF(S471A) MEK1-binding mutant also presented as predominantly a single lower band that was insensitive to MS934 like the CRAF(K375M) or CRAF(S621A) mutants (FIGS. 12H, 19F-G). Depletion of Flag-CRAF(S471A) levels was detected with cycloheximide alone, demonstrating that in the absence of MEK1-binding, CRAF(S471A) predominantly existed as the immature form of CRAF. The results show that HSP90 inhibition reduced both forms of Flag-CRAF(WT) and immature Flag-CRAF(S621A), Flag-CRAF(K375M) or FLAG-CRAF(S471A) mutant protein levels (FIG. 12I).

Together, these findings demonstrate that CRAF mutants unable to autophosphorylate at S621 or that exhibit impaired MEK1-binding were insensitive to MS934, predominately existed as the immature form of CRAF, and were more sensitive to protein synthesis inhibition than mature CRAF.

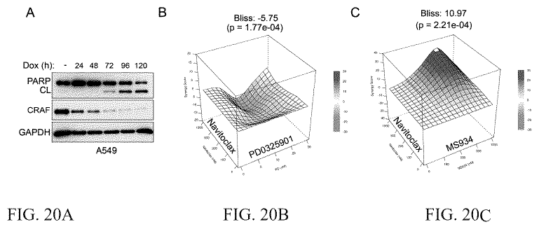

Immature CRAF Has Anti-Apoptotic Functions Independent of MEK1/2. CRAF has been shown to have kinase-independent functions outside the MEK1/2 signaling axis (McCormick, F. 2018. Cancer Cell, 33, 158-159). CRAF promotes cell survival by inhibiting apoptosis through a kinase-independent mechanism involving binding to apoptosis signal-regulating kinase 1 (ASK1) (Chen, J., et al. 2001. Proceedings of the National Academy of Sciences, 98, 7783-7788). Notably, CRAF knockdown by shRNA in HCT-116 and A549 cells caused an increase in cleaved PARP, indicating activation of caspase activity and apoptosis (FIGS. 13A-B). A time course analysis of CRAF knockdown in A549 cells showed that CRAF protein levels were sufficiently reduced to promote cleaved PARP by 72 hours post-doxycycline induction (FIG. 20A). In contrast, depletion of the mature form of CRAF and MEK1/2 by 1 mM MS934 treatment minimally increased cleaved PARP levels compared to CRAF knockdown (FIGS. 13A-B). Strikingly, knockdown of the residual immature CRAF post-MS934 treatment in HCT116 or A549 cells by shRNA induced apoptosis by 48 hours (FIGS. 13C-D), showing that the inability of MS934 to induce apoptosis could be due to insufficient blockade of CRAF anti-apoptotic functions outside the MEK-ERK pathway. Consistent with this, immunoprecipitation of Flag-CRAF in MS934-treated PANC-1(MEK1-GFP) cells revealed an increased interaction between ASK1 and residual CRAF compared to vehicle treatment (FIG. 13E). A similar phenomenon was observed with immature kinase dead Flag-CRAF(K375M) (FIG. 13E). Together, these findings demonstrated that post-MEK1/2 degradation the residual immature CRAF promoted survival outside the MEK1/2 pathway via binding and antagonizing ASK1.

An ASK1/Jun N-terminal protein kinase pathway phosphorylates B-cell lymphoma 2 (BCL2) inactivating its anti-apoptotic function promoting cell death (Yamamoto, K., et al. 1999. Mol Cell Biol, 19, 8469-78). As immature CRAF blocks apoptosis via inhibiting ASK1, it was tested whether inhibition of BCL2 in MS934-treated cells would enhance caspase activation. Indeed, treatment of HCT-116 or A549 cells with the combination of the BCL2 inhibitor, Navitoclax, and MS934 enhanced caspase-3/7 activation compared to single agent treatments (FIGS. 13F-G). Notably, blocking BCL2 function in MS934-treated cells was more effective at activating caspase 3/7 than combining Navitoclax and PD0325901. Moreover, the combination of Navitoclax and MS934 blocked cell viability (FIG. 20B-C) and anchorage-independent colony growth of HCT-116 and/or A549 cells to a greater extent than single agent treatments or the combination of Navitoclax and PD0325901 (FIGS. 13H-I).

A model for drug synergy combining MEK1/2 degraders with BCL2 inhibitors is shown in FIG. 22. In MEK1/2 competent cells, a ASK1/JNK signaling pathway negatively regulates BCL2 function promoting apoptosis. Under conditions of MEK1/2 degradation, accumulation of immature CRAF occurs leading to inhibition of ASK1/JNK activity and increased dependency on anti-apoptotic functions of BCL2. MS934-treated cells acquire enhanced sensitivity to BCL2 inhibition using inhibitors such as Navitoclax, where co-treatment of cells with MS934+Navitoclax causes increased apoptosis and blockade of cell viability. This drug combination could be used to treat cancer, including cancers with altered MAPK signaling. MS934 combined with Navitoclax will be tested. For example, cancer cell line xenograft model will be treated with the MS934 and Navitoclax combination and the tumor growth response will be monitored.

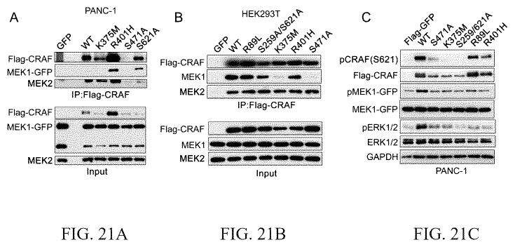

A stable complex of CRAF-MEK1/2 exists that is primed for activation by RAS binding and RAF dimerization. To define when MEK1/2 binding to CRAF occurs in the process of CRAF maturation, various Flag-CRAF mutants were expressed in PANC-1(MEK1-GFP) or HEK293T cells, immunoprecipitated Flag, and probed for MEK1 and MEK2 interactions. Both MEK1-GFP and endogenous MEK1 interacted with Flag-CRAF(WT), Flag-CRAF(R89L) (impaired RAS binding), Flag-CRAF(R401H) (impaired dimerization), Flag-CRAF(S621A) and Flag-CRAF(S259A/S621A), whereas the Flag-CRAF(S471A) and Flag-CRAF(K375M) mutants showed reduced binding to MEK1 (FIGS. 14A, 21A-B). Endogenous MEK2 interacted with the Flag-CRAF mutants, shower there may be differences in how MEK1 and MEK2 interact with CRAF (FIGS. 14A, 21A-B). Notably, pulldown of MEK1/2 with the Flag-CRAF(S259A/S621A) or Flag-CRAF(S621A) mutants revealed that MEK1/2 binds to CRAF independent of p-S621 status. Additionally, it was observed that HSP90 and CDC37 interacted with the Flag-CRAF constructs, while 14-3-3 interacted with Flag-CRAF(WT), Flag-CRAF(R89L), Flag-CRAF(R401H) and minimally with Flag-CRAF(S471A) (FIG. 14A). BRAF was detected bound to Flag-CRAF(WT) and weakly to Flag-CRAF(R89L) with minimal BRAF interaction observed with the other CRAF mutants (FIG. 14A), demonstrating some CRAF-BRAF dimerization occurs in the absence of CRAF-based RAS binding.

To determine if MEK1/2 binding to Flag-CRAF(R89L), or Flag-CRAF(R401H) preserved p-S621 and protein stability like wild-type, cells were treated with MS934, cycloheximide, or the combination and measured Flag-CRAF and p-S621 levels. MS934 treatment reduced p-S621 and total protein levels of Flag-RAF(R89L) or Flag-CRAF(R401H), and the combination of MS934 and cycloheximide enhanced degradation of the Flag-CRAF(R89L) or Flag-CRAF(R401H) mutants as observed for Flag-CRAF(WT) (FIG. 14B). Together, the MEK1/2 data show that 1) MEK1/2 interaction with CRAF occurs before RAS binding and/or RAF dimerization to promote phosphorylation of p-S621 and stabilization of total CRAF protein levels and 2) a primed complex of CRAF-MEK1/2 (including 14-3-3-HSP90-CDC37) forms in cells independent of RAF dimerization and before RAS interaction.

Next, it was assessed whether RAS binding or RAF dimerization was important to activate the CRAF-MEK1/2 complex. Co-expression of Myc-MEK1/2 and Flag-CRAF(R89L) or Flag-CRAF (R401H) in PANC-1 or HEK293T cells increased Flag-CRAF protein and p-S621 levels like Flag-CRAF(WT), whereas no change in Flag-CRAF protein or p-S621 was observed with the kinase-dead Flag-CRAF(K375M) mutant (FIG. 14C). The results show that expression of Flag-CRAF(WT) with Myc-MEK1/2 increased p-MEK1/2 in PANC-1 and HEK293T cells. Increased levels of p-MEK1/2 were also detected following expression of the Flag-CRAF(R89L) mutant with Myc-MEK1/2, consistent with prior studies demonstrating that MEK1 activates CRAF through a RAS-independent mechanism (Leicht, D. T., et al. 2013. Biochimica et Biophysica Acta (BBA) —Molecular Cell Research, 1833, 976-986). Notably, the phosphorylation of MEK1/2 by the Flag-CRAF(WT) was significantly greater than observed for the Flag-CRAF(R89L) mutant in PANC-1 and HEK293T cells (FIGS. 14D-E). No change in p-MEK1/2 levels was observed with the Flag-CRAF(R401H) resembling the kinase-dead Flag-CRAF(K375M) mutant. Similarly, expression of Flag-CRAF(R89L) or Flag-CRAF(R401H) in PANC-1(MEK1-GFP) cells did not activate MEK or ERK like Flag-CRAF(WT) (FIG. 21C). Together, these findings show that the CRAF-MEK1/2 complex required RAS interaction and RAF dimerization for optimal activation of CRAF and subsequent phosphorylation of MEK1/2.

Phosphorylation of CRAF by ERK1/2 negatively regulates the interaction between CRAF and RAS, controlling CRAF activity (Dougherty, M. K., et al. 2005. Molecular Cell, 17, 215-224). MEK inhibition with inhibitors such as PD0325901 increases CRAF and MEK1 interactions due to alleviation of ERK1/2 negative feedback on CRAF (Lito, P., et al. 2014. Cancer Cell, 25, 697-710). The results described herein show that directly inhibiting ERK1/2 activity using SCH772984 in HCT-116 cells reduces ERK-regulated sites p-S289/296/301 promoting increased interaction between MEK1 and CRAF (FIG. 14E). These findings demonstrate that ERK1/2 activity could control formation of a primed CRAF-MEK complex, competent to create a RAS-RAF-MEK signalosome.

Collectively, these findings support a revised model of CRAF activation (FIG. 14F) in which MEK1/2 binding to newly synthesized immature CRAF, along with HSP90-CDC37, promotes phosphorylation at S621, and protein stabilization. A CRAF-MEK-HSP90-CDC37-14-3-3 complex forms, primed for activation by RAS binding. Upon RAS engagement, CRAF dimerizes with BRAF, forming an active signalosome whereby RAFs phosphorylate MEK, leading to ERK activation and promoting cell growth. Once phosphorylated, MEK1/2 could dissociate from CRAF promoting dephosphorylation of S621 and subsequent CRAF degradation. Binding of the CRAF-MEK complexes to RAS, as well as CRAF-MEK complex formation, are negatively regulated by active ERK1/2. Finally, although the majority of CRAF in cells is the mature form, a minor pool of immature CRAF exists in cells that has anti-apoptotic functions and can also dimerize with ARAF/BRAF to promote MAPK signaling.

Kinases are largely defined by their catalytic function despite many possessing roles independent of their kinase activity (Rauch, J., et al. 2011. Cell Communication and Signaling, 9, 23). Regarding MEK1/2, the canonical function is the phosphorylation of ERK1/2 in the MAPK cascade. Such roles have been confirmed using kinase inhibitors, which assess catalytic function. Not surprisingly, many enzymes, including kinases, are involved in structured protein complexes but their involvement in the stability of the complex remains understudied. Here, using MEK1/2 PROTACs, a new kinase-independent function of MEK1/2 was identified whereby MEK1/2 regulate the stability of the upstream kinase CRAF, but not the related ARAF or BRAF proteins. MEK1/2 facilitate the maturation of CRAF to an active kinase allowing initiation of the MAPK cascade via dimerization with ARAF/BRAF in a RAS-dependent manner. Without MEK1/2, a pool of immature CRAF maintains its anti-apoptotic functions.

CRAF is comprised of two cellular pools, an immature form devoid of kinase activity, and the mature active form harboring p-S621 that potentiates the MAPK signaling cascade (Mitra, S., et al. 2016. J Biol Chem, 291, 24579-24593). The maturation of CRAF is orchestrated by chaperones CDC37 and HSP90, which ensure proper folding of newly synthesized CRAF rendering the kinase capable to induce autophosphorylation of 5621 and subsequent maturation (Silverstein, A. M., et al. 1998. J Biol Chem, 273, 20090-5; Grammatikakis, N., et al. 1999. Mol Cell Biol, 19, 1661-72; Smith, J. R., et al. 2009. Oncogene, 28, 157-69; Mitra, S., et al. 2016. J Biol Chem, 291, 24579-24593; and Garcia-Alonso, S., et al. 2022. Mol Cell, 82, 3438-3452 e8). HSP90 inhibitors selectively target immature CRAF causing its degradation, while exhibiting no effect on mature CRAF. In contrast, as described herein, it was discovered that MEK1/2 degradation caused preferential loss of mature active CRAF. These findings illustrate the importance of both HSP90 and MEK1/2 in CRAF maturation. Interestingly, both MEK1/2 and HSP90/CDC37 bind the immature Flag-CRAF(S259A/S621A) mutants, demonstrating that MEK1/2 can associate with CRAF independent of S621 phosphorylation status. HSP90 inhibition or MEK1/2 degradation caused loss of CRAF p-S621 levels, demonstrating MEK1/2 binding and HSP90 activity are both important for CRAF autophosphorylation at 5621. Moreover, the binding of HSP90/CDC37 to residual CRAF devoid of S621 phosphorylation post-MS934 treatment and the impaired MEK1/2 binding CRAF(S471A) mutant indicate that HSP90/CDC37 chaperone activity is not sufficient to promote CRAF autophosphorylation at S621 in the absence of MEK1/2 binding.

Bystander proteins interacting with PROTAC targets have been reported to be collaterally degraded due to being in proximity of the recruited E3 ubiquitin ligase and proteasome machinery (Xiong, Y., et al. 2022. J Am Chem Soc, 144, 22622-22632). A detailed temporal evaluation of CRAF ubiquitination and degradation revealed that CRAF was degraded via a conserved cell-intrinsic mechanism rather than collateral degradation. The E2/E3 ligases responsible for promoting the degradation of CRAF in response to MEK1/2 degradation have not been identified, but if they were, could facilitate the development of CRAF degraders or molecular glues. It is likely that there are no published CRAF degraders because most RAF inhibitors target the active form of CRAF (Singh, A. K., et al. 2023. ACS Omega, 8, 27819-27844; and Shao, W., et al. 2018. Cancer Res, 78, 1537-1548). Degraders targeting immature CRAF will be most advantageous, as these agents will deplete both immature and mature forms of CRAF, leading to apoptosis. HSP90 inhibitors promote degradation of the immature form of CRAF but are not selective given that HSP90 has hundreds of client proteins (Kamal, A., et al. 2004. Trends Mol Med, 10, 283-90. Alternatively, knockdown of the chaperone CDC37 promotes CRAF protein loss (Smith, J. R., et al. 2009. Oncogene, 28, 157-69). Structural investigation of the CDC37-CRAF interface demonstrates that its disruption affects CRAF protein stability, highlighting the need for inhibitors or “busters” of this interaction that would selectively target immature CRAF, sparing other CDC37/HSP90 client proteins (Garcia-Alonso, S., et al. 2022. Mol Cell, 82, 3438-3452 e8.

The current CRAF activation cycle places MEK1/2 recruitment following RAS binding and CRAF dimerization (Lavoie, H. & Therrien, M. 2015. Nature Reviews Molecular Cell Biology, 16, 281-298. As described herein, the results demonstrate that MEK1/2 binding to CRAF occurs before RAS engagement and RAF dimerization, whereby MEK1/2 kinase-independent functions promote CRAF protein phosphorylation, stabilization, and maturation. Upon MEK1/2 depletion, CRAF is rapidly dephosphorylated at S621 by PP1/2 phosphatases, impairing 14-3-3 binding to CRAF, promoting its degradation. Prior studies showed that MEK1 activates CRAF through a RAS-independent mechanism that required MEK1-binding (Leicht, D. T., et al. 2013. Biochimica et Biophysica Acta (BBA)—Molecular Cell Research, 1833, 976-986). Described herein, the results show that CRAF(R89L) RAS binding mutant can promote some MEK1/2 and ERK1/2 phosphorylation, though substantially less than that observed with wild-type CRAF. Notably, immunoprecipitation of the Flag-CRAF(R89L) mutant detected interaction with BRAF, demonstrating that activation of the MAPK pathway by the Flag-CRAF(R89L) could involve dimerization of a preformed CRAF-MEK complex with BRAF. This is likely, as co-expressing the CRAF(R401H) dimerization mutant with MEK1/2 caused minimal phosphorylation of MEK, signifying that dimerization of CRAF was important to achieve MEK1/2 phosphorylation. While it cannot be excluded that dimerization of immature CRAF with BRAF could substitute for MEK chaperone function in forming an active signalosome, the results disclosed herein demonstrate that MEK1/2 binding to CDC37/HSP90-bound CRAF promotes CRAF protein stabilization and maturation, and that a primed complex of CRAF-MEK is formed that requires RAS binding and RAF dimerization to form a fully active signalosome to achieve optimal phosphorylation of MEK1/2.

Direct negative feedback phosphorylation of CRAF at sites within its RAS binding domain by ERK1/2 releases CRAF from RAS, dissociating RAF dimers and inhibiting the MAPK cascade, with dephosphorylation of CRAF by PP2A and PIN1 promoting MAPK reactivation (Dougherty, M. K., et al. 2005. Molecular Cell, 17, 215-224). As described herein, the results show that binding of MEK1/2 to CRAF promotes stabilization and maturation, but how the recruitment of MEK to CRAF is regulated to promote CRAF activation remains undetermined. MEK1/2 blockade by inhibitors (i.e., PD0325901) relieves ERK-mediated feedback inhibition of CRAF promoting CRAF-MEK protein interaction (Lito, P., et al. 2014. Cancer Cell, 25, 697-710), showing that ERK1/2 activity may directly regulate MEK1/2-CRAF complex formation. Consistent with this, the results also show that the ERK1/2 inhibitor SCH772984 promoted an association of CRAF with MEK1. These findings demonstrate that activated ERK1/2 may negatively regulate the interaction of CRAF and MEK1, and that once ERK1/2 is inactivated by dephosphorylation, MEK1/2 binds to CRAF promoting maturation and activation of CRAF in a new primed complex. Interestingly, stoichiometric analysis of MEK1 and CRAF levels in cells showed that there is 100-fold more MEK than CRAF in cells (Fujioka, A., et al. 2006. J Biol Chem, 281, 8917-26); yet CRAF activity is tightly controlled. Notably, proteins such as BRAF or KSR can sequester MEK from CRAF by dimerizing kinase domain to kinase domain; similarly MEK-MEK dimerization (Catalanotti, F., et al. 2009. Nat Struct Mol Biol, 16, 294-303) could limit CRAF-MEK complex formation in quiescent cells (Haling, J. R., et al. 2014. Cancer Cell, 26, 402-413; and Catalanotti, F., et al. 2009. Nat Struct Mol Biol, 16, 294-303).

Despite similar structures and functions, CRAF and BRAF exhibit opposing behaviors in response to MEK1/2 degradation, (i.e., dephosphorylation and degradation of CRAF and activation of BRAF). Mutation of the S621 autophosphorylation site to an alanine or kinase inactivating mutations renders CRAF proteolytically unstable resulting in increased degradation (Noble, C., et al. 2008. Mol Cell, 31, 862-72). In contrast, kinase-dead BRAF did not impact BRAF protein stability, signifying BRAF and CRAF stability and activation are differentially regulated in cells (Noble, C., et al. 2008. Mol Cell, 31, 862-72). Notably, BRAF harbors evolutionarily conserved aspartic residues in its N-terminal region that mimic phosphorylation producing a constitutively active kinase, whereas CRAF requires phosphorylation at several sites to become an active kinase (Mason, C. S., et al. 1999. Embo j, 18, 2137-48). BRAF also does not require the complex orchestration of chaperone activity associated with CRAF as demonstrated by HSP90 inhibition (Grbovic, O. M., et al. 2006. Proc Natl Acad Sci USA, 103, 57-62). Like MEK1/2 inhibition (Lito, P., et al. 2014. Cancer Cell, 25, 697-710), it was found that degradation of MEK1/2 promoted BRAF dimerization with residual immature CRAF activating BRAF, signifying that immature CRAF, devoid of kinase activity, can promote MAPK signaling. The findings described herein are consistent with studies that showed CRAF dimerization rather than activity promotes KRAS tumorigenesis (Venkatanarayan, A., et al. 2022. Cell Rep, 38, 110351). Activation of BRAF by MS934 will likely be a resistance mechanism, requiring co-inhibition of BRAF, with agents like RAF709, with MEK1/2 degraders to sustain suppression of MEK-ERK activation. BRAF and MEK1 form an inactive complex that is recruited to the membrane upon RAS activation, promoting BRAF dimerization and activation (Haling, J. R., et al. 2014. Cancer Cell, 26, 402-413). Without MEK1/2 proteins, BRAF is free from MEK1 regulation and able to dimerize with immature CRAF promoting BRAF activation. Similar findings regarding BRAF activation showed that MEK1 binding to KSR1 promotes BRAF-KSR1 dimerization independent of MEK1 kinase activity (Lavoie, H., et al. 2018. Nature, 554, 549-553). Thus, MEK1/2 binding to BRAF may negatively regulate BRAF dimerization with CRAF, controlling BRAF activation. In contrast, MEK1/2 binding dictates CRAF stability and activation.

MS934 promotes the degradation of both MEK1 and MEK2, causing subsequent loss of CRAF in cells. Genetic knockdown of endogenous MEK1 or MEK2 alone did not cause CRAF degradation, demonstrating that both MEK1 and MEK2 binding to CRAF are required for CRAF stabilization, phosphorylation, and maturation. Interestingly, the results disclosed herein demonstrate that MEK1 and MEK2 have differential binding preferences for CRAF mutants, finding MEK2, but not MEK1, bound to CRAF(S471A) and CRAF(K375M). Mutation of lysine 375 blocks interaction with MEK1 in assays with purified proteins, demonstrating CRAF ATP-binding was necessary for MEK1/2 binding (Xiang, X., et al. 2002. J Biol Chem, 277, 44996-5003). MEK1 binding to CRAF is likely kinase domain to kinase domain, whereby disruption of the catalytic lysine in CRAF could disrupt kinase domain interactions. How the S471A mutation impairs MEK1 binding to CRAF has not been determined. The fact that MEK2 can bind the CRAF(K375M) or CRAF(S471A) mutants suggests MEK2 may interact with a region of CRAF distinct from MEK1, allowing the possibility for simultaneous binding of MEK1 and MEK2 to wild-type CRAF. Further molecular and structural studies defining the MEK1 and MEK2 binding sites within CRAF, and whether MEK2 functions differently than MEK1 in the stabilization and maturation of CRAF will be of particular interest. MEK2 has an important role in apoptosis (Huth, H. W., et al. 2016. Cellular Signalling, 28, 1283-1291), while MEK1 serves as the predominant MEK in RAS-driven MAPK signaling (Chen, J., et al. 2001. Proceedings of the National Academy of Sciences, 98, 7783-7788), suggesting MEK2 may regulate the anti-apoptotic function of immature CRAF.

CRAF has both kinase-dependent and kinase-independent functions through activation of MEK-ERK signaling and promoting growth and survival of KRAS mutant cancers, respectively (McCormick, F. 2018. Cancer Cell, 33, 158-159). Genetic depletion of CRAF but not its inhibition leads to growth repression and apoptosis in KRAS mutant cancers (Sanclemente, M., et al. 2021. Cancer Cell, 39, 294-296; and Venkatanarayan, A., et al. 2022. Cell Rep, 38, 110351). Kinase-independent functions of CRAF suppress apoptosis in KRAS mutant cells via interaction with pro-apoptotic kinases ASK1 and MST2, inhibiting their kinase activities (O'Neill, E., et al. 2004. Science, 306, 2267-70; and CHEN, J., et al. 2001. Proceedings of the National Academy of Sciences, 98, 7783-7788). Consistent with these findings, the results described herein showed that without MEK1/2 present the immature form of CRAF exhibits anti-apoptotic functions via ASK1 and BCL2 (Yamamoto, K., et al. 1999. Mol Cell Biol, 19, 8469-78). Moreover, the results demonstrate that MEK1/2 degradation promotes dependency on immature CRAF's anti-apoptotic function, which sensitizes MS934-treated cells to BCL2 inhibition. It will be of particular interest to determine whether the anti-apoptotic functions of residual immature CRAF will promote resistance and disease persistence following chronic MS934 exposure. Additionally, deconvoluting interacting partners of immature CRAF and mature CRAF could reveal functions of immature CRAF outside of MEK-ERK signaling. Kinase-independent functions of CRAF have been implicated in DNA damage (Advani, S. J., et al. 2015. Nat Commun, 6, 8154) and cell cycle progression (Mielgo, A., et al. 2011. Nat Med, 17, 1641-5). Whether these roles require MEK1/2 binding or are orchestrated by immature CRAF remains to be determined.

CRAF degradation represents a highly advantageous therapeutic target for the treatment of MAPK-altered cancers; however, there are no published CRAF degraders. The findings described herein show that developing CRAF degraders may prove difficult using existing RAF inhibitors due to the complex maturation process of CRAF. Here, an alternative strategy was identified to deplete the majority of CRAF protein in cells by exploiting the kinase-independent function of MEK1/2 in preserving CRAF protein stability. These findings support the use of MEK1/2 degraders combined with translation inhibitors, such as MTOR inhibitors, to degrade both immature and mature CRAF or targeting the anti-apoptotic functions of immature CRAF using BCL2 inhibitors. Notably, it has been shown that MS934 treatment was safely administered in vivo and blocked tumor growth when combined with EGFR inhibitors in KRAS mutant colorectal cancer xenografts (Kurimchak, A. M., et al. 2022. Sci Signal, 15, eabn2707.

Experimental Procedures. Cell Lines. Cell lines were verified by IDEXX laboratories and free of mycoplasma. The media with the exception of the Mammary Epithelial Cell Basal Medium was supplemented with 10% FBS, 100 U/ml Penicillin-Streptomycin and 2 mM GlutaMAX. HCT-116, NCI-H23, A549, HT-29, and OVCAR8 cell lines were maintained in RPMI-1640. HEK293T cells were maintained in DMEM. PANC-1 and HPAF-II cells were maintained in DMEM supplemented with 1 mM Sodium Pyruvate. SKCO1 cells were maintained in MEM supplemented with 1 mM Sodium Pyruvate. Capan-1 cells were maintained in Iscove's MDM. Calu-1 cells were maintained in McCoy's 5a medium. MCF10A cells were grown in ATCC's Mammary Epithelial Cell Basal Medium (ATCC PCS-600-030) supplemented with the Mammary Epithelial Cell Growth Kit (ATCC PCS-600-040) All cells were kept at 37° C. in a 5% CO₂ incubator.

Compounds. MEK1/2 degraders MS432, MS910 and MS934 were used (Hu, J., et al. 2020. J Med Chem, 63, 15883-15905; and Wei, J., et al. 2019. Journal of Medicinal Chemistry, 62, 10897-10911). The other compounds are listed in Table 1.

TABLE 1
Compounds

| Inhibitor | Manufacturer | Cat # |
|---|---|---|
| PD0325901 | Selleckchem | S1036 |
| Bortezomib | Selleckchem | S1013 |
| Ganetespib (STA-9090) | Selleckchem | S1159 |
| GDC-0973 | MedChemExpress | HY-13064 |
| MAP855 | MedChemExpress | HY-145702 |
| NST-628 | MedChemExpress | HY-158115 |
| RO5126766 | Selleckchem | S7170 |
| SCH772984 | MedChemExpress | HY-50846 |
| Cycloheximide | Sigma Aldrich | 239763-1GM-M |
| Trametinib | Selleckchem | S2673 |
| Calyculin A | MedChemExpress | HY-18983 |
| Navitoclax | Selleckchem | S1001 |
| RAD001 | Selleckchem | S1120 |
| Torin-1 | Selleckchem | S2827 |

Plasmids. pLKO-Tet-puro-hRAF1-shRNA-1 (Addgene plasmid #185371; n2t.net/addgene:185371; RLRID:Addgene_185371), MVEK1-GFP (Addgene plasmid #14746; http://n2t.net/addgene:14746; RIRID:Addgene_14746) pEF-MYC-RAF1, Myc-Mek1 was a gift from Dustin Maly (Addgene plasmid #40774; n2t.net/addgene:40774; RRID:Addgene_40774); Myc-Mek2 (Addgene plasmid #40776; n2t.net/addgene:40776; RRID:Addgene_40776), mCherry-RAF1 (EX-I5873-Lv1111-RAF1), Flag-RAF1 (EX-T0483-M11) Flag-BRAF (EX-Z0707-M11), and Flag-ALRAF (EX-B0257-M11) were used (purchased from Genecopoeia). Mutations were made using the QuikChange XL Site-Directed Mutagenesis Kit (Agilent) according to manufacturer's protocol. A list of the mutagenic primers is in Table 2.

TABLE 2
Mutagenic primers.

| Protein | Forward | Reverse |
|---|---|---|
| RAF1 | 5′-TTATGAAAGCACTCAAGGTG | 5′-CAGCACTCTGGTTGCAGGCCTAGC |
| R89L | CTAGGCCTGCAACCAGAGTG | ACCTTGAGTGCTTTCATAA-3′ |
| | CTG-3′ | (SEQ ID NO: 2) |
| | (SEQ ID NO: 1) | |
| RAF1 | 5′-CCAGAGGCAGAGGTCGA | 5′-TTAGGTGTGGCTGTCGACCTCT |
| S259A | CAGCCACACCTAA-3′ | GCCTCTGG-3′ |
| | (SEQ ID NO: 3) | (SEQ ID NO: 4) |
| RAF1 | 5′-GCACGGAGATGTTGCAG | 5′-CAACCTTTAGGATCATTACTGC |
| K375M | TAATGATCCTAAAGGTTG-3′ | AACATCTCCGTGC-3′ |
| | (SEQ ID NO: 5) | (SEQ ID NO: 6) |
| RAF1 | 5′-TCAACCGGAGCGCTGCC | 5′-GGATGGCTCGGCAGCGCTCCG |
| S621A | GAGCCATCC-3′ | GTTGA-3′ |
| | (SEQ ID NO: 7) | (SEQ ID NO: 8) |
| RAF1 | 5′-GGCTGTTCTGCGCAAAA | 5′-AAAAGCAGAATGTTCACATGA |
| R401H | CACATCATGTGAACATTCTG | TGTGTTTTGCGCAGAACAGCC-3′ |
| | CTTTT-3′ | (SEQ ID NO: 10) |
| | (SEQ ID NO: 9) | |
| RAF1 | 5′-GAACATCATCCATAGAGA | 5′-ATGGAGAAATATATTGTTGGCTTT |
| S471A | CATGAAAGCCAACAATATAT | CATGTCTCTATGGATGATGTTC-3′ |
| | TTCTCCAT-3′ | (SEQ ID NO: 12) |
| | (SEQ ID NO: 11) | |
| MEK1 | 5′-GGCCTGGTCATGGCCAGA | 5′-CAGATGAATTAGCATTCTGGCCAT |
| K97M | ATGCTAATTCATCTG-3′ | GACCAGGCC-3′ |
| | (SEQ ID NO: 13) | (SEQ ID NO: 14) |
| MEK1 | 5′-CGGAATGGACAGCCGACCTC | 5′-TATGTAATCCAACAACTCAAAAGC |
| IRI | CCGCGGCAGCTTTTGAGTTG | TGCCGCGGGAGGTCGGCTGTCCAT |
| | TTGGATTACATA-3′ | TCCG-3′ |
| | (SEQ ID NO: 15) | (SEQ ID NO: 16) |
| MEK2 | 5′-CTCATCATGGCCAGGATGC | 5′-CAAGGTGGATCAGCATCCTGGCC |
| K101M | TGATCCACCTTG-3′ | ATGATGAG-3′ |
| | (SEQ ID NO: 17) | (SEQ ID NO: 18) |
| MEK2 | 5′-GGATAGCCGGCCTGCCGC | 5′-GTCCAGGAGTTCAAAGGCGGCCG |
| IRI | GGCCGCCTTTGAACTCCTGG | CGGCAGGCCGGCTATCC-3′ |
| | AC-3′ | (SEQ ID NO: 20) |
| | (SEQ ID NO: 19) | |
| MEK1 | 5′-GAGACCAACTTGGAGGCC | 5′-CAAGCTCTAGCTCCTCTAATTTTTT |
| siRNA-Resistant | TTGCAGAAAAAATTAGAGG | CTGCAAGGCCTCCAAGTTGGTCTC-3′ |
| | AGCTAGAGCTTG-3′ | (SEQ ID NO: 22) |
| | (SEQ ID NO: 21) | |

Western Blotting. Samples were harvested in lysis buffer (50 mM HEPES (pH 7.5), 0.500 Triton X-100, 150 mM NaCl, 1 mM EDTA, 1 mM EGTA, 10 mM sodium fluoride, 2.5 mM sodium orthovanadate, 1× protease inhibitor cocktail (Roche), and 1× each of phosphatase inhibitor cocktails 2 and 3 (Sigma)). Particulate was removed by centrifugation of lysates at 21,000 rpm for 15 minutes at 4° C. Lysates were subjected to SDS-PAGE chromatography and transferred to PVDF membranes before western blotting with primary antibodies (Table 3). Secondary HRP-anti-rabbit and RP-anti-mouse were obtained from ThermoFisher Scientific. SuperSignal West Pico and Femto Chemiluminescent Substrates (Thermo) were used to visualize blots. Western blot images were quantified using the Analyze>Gels function in Image J open-source software (National Institutes of Health). Optical density values for total protein levels were normalized by GAPDH. DC50 (the concentration where 5000 of protein has been degraded) plots and values were calculated using PRISM software. Samples were run in biological triplicates (N=3) and Student's t-tests were performed for statistical analyses and P values ≤0.05 were considered significant.

TABLE 3
Antibodies.

| Antibody | Manufacturer | Cat # |
|---|---|---|
| pARAF S299 | CST | 4431 |
| ARAF | SCBT | sc-166771 |
| ARAF | CST | 4432 |
| pBRAF S365 | Biorbyt | orb5749 |
| pBRAF S445 | CST | 2696 |
| pBRAF S729 | Abcam | ab124794 |
| BRAF | CST | 14814 |
| pCRAF S259 | CST | 9421 |
| pCRAF S338 | CST | 9427 |
| pCRAF S621 | SCBT | sc-271929 |
| pCRAF S289/296/301 | CST | 9431 |
| CRAF | CST | 9422 |
| CRAF | Abcam | ab181115 |
| pMEK S217/221 | CST | 9154 |
| MEK1 | CST | 12671 |
| MEK2 | SCBT | sc-524 |
| pERK T202/204 | CST | 4370 |
| ERK1/2 | CST | 4696 |
| GAPDH | CST | 2118 |
| ASK1 | CST | 8662 |
| HSP90 | CST | 4877 |
| 14-3-3 | CST | 95422 |
| CDC37 | CST | 4793 |
| PARP | CST | 9542 |
| Cul2 | Abcam | ab166917 |
| VHL | CST | 68547 |
| Myc 9e10 | SCBT | sc-40 |
| Flag(M2) | Sigma | F1804 |
| DYKDDDDK Tag (SEQ ID | CST | 14793 |
| NO: 23) | | |

Immunoprecipitations. For Myc- and Flag-tagged protein IPs, cells were transfected 48 hours prior to harvest using Lipofectamine 3000 according to manufacturer's instructions. Cells were harvested in a buffer containing 25 mM TrisCl (pH 7.5), 1% NP-40, 150 mM NaCl, 1 mM EDTA, 5% glycerol, 1× protease inhibitor cocktail (Roche), and 1× each of phosphatase inhibitor cocktails 2 and 3 (Sigma). Lysates were incubated for 1 hour using over-end rotation, then centrifuged at 21,000 rpm for 15 minutes at 4° C. 1 mg protein was added to 10 μl anti-c-myc or anti-DYKDDDDK (SEQ ID NO: 23) magnetic beads (Pierce) and incubated for 2 hours using over-end rotation. Beads were washed 4× with lysis buffer, then boiled in 2×LSB at 100° C. for 5 minutes. For endogenous IPs, cells were harvested as above, then 1 mg protein was precleared using normal mouse IgG agarose conjugate (Santa Cruz) for 2-4 hours at 4° C. Following pre-clearing, lysates were added to 20 μl ARAF, BRAF, RAF1 or MEK1 agarose conjugate (Santa Cruz, Table 4) or normal mouse IgG agarose conjugate and incubated overnight. Beads were washed and proteins eluted as above. TUBE1-IPs were performed (Alabi, S., et al. 2021. Nature Communications, 12, 920). Briefly, cells were harvested in buffer containing 50 mM Tris-HCl, pH 7.4, 0.15 M NaCl, 1 mM EDTA, 1% NP40, 10% glycerol, 5 mM 1,10-phenanthroline monohydrate, 10 mM N-ethylmaleimide, 20 μM PR-619, 1× protease inhibitor cocktail (Roche) and 1× each of phosphatase inhibitor cocktails 2 and 3 (Sigma). 1 mg protein was added to 20 μl of TUBE1 agarose (LifeSensors) and incubated for 2 hours at 4° C. Beads were washed 4 times in Tween-TBS and boiled in 2×LSB at 100° C. for 5 minutes.

TABLE 4
Aragrose conjugates.

| Agarose and Magnetic | | |
|---|---|---|
| Conjugates | Manufacturer | Cat # |
| normal mouse IgG agarose | SCBT | sc-2343 AC |
| conjugate | | |
| RAF-1 (E-10) agarose | SCBT | sc-7267 AC |
| conjugate | | |
| MEK1 (H-8) agarose | SCBT | sc-6250 AC |
| conjugate | | |
| BRAF (F-7) agarose | SCBT | sc-5264 AC |
| conjugate | | |
| ARAF (A-5) agarose | SCBT | sc-166771 AC |
| conjugate | | |
| anti-c-myc magnetic beads | Pierce | 88842 |
| anti-DYKDDDDK (SEQ ID | Pierce | A36797 |
| NO: 23) magneic beads | | |
| anti-GFP magnetic beads | CST | 67090 |

*SCBT = Santa Cruz Biotechnology
*CST = Cell Signaling Technology

qRT-PCR. GeneJET RNA purification kit (Thermo Scientific) was used to isolate RNA from cells according to manufacturer's instructions. qRT-PCR on diluted cDNA was performed with inventoried TaqMan Gene Expression Assays on the Applied Biosystems 7500 Fast Real-Time PCR System. The TaqMan Gene Expression Assay probes (ThermoFisher Scientific) used to assess changes in gene expression include RAF1 (Assay ID: Hs00234119_ml), and ACTB (control) (Cat #4326315E). Samples were run in biological triplicates (N=3) and Student's t-tests were performed for statistical analyses and P values ≤0.05 were considered significant.

RNAi-mediated knockdown. siRNA transfections were performed using 25 nM siRNA duplex and the reverse transfection protocol. Cells were added to plates with media containing the siRNA (Table 5) and transfection reagent (Lipofectamine RNAiMax) according to the manufacturer's instructions. Cells were collected 72 h post-transfection. Medium was changed and drug was added 24 h prior to collection. Two to three independent experiments were performed with each cell line and siRNA. Samples were run in biological triplicates (N=3) and Student's t-tests were performed for statistical analyses and P values ≤0.05 were considered significant.

TABLE 5
siRNA.

| SIRNA | Cat. # | Manufacturer |
|---|---|---|
| siGenome SmartPool | M-003571-01 | Horizon Discovery (GE |
| MAP2K1 | | Dharmacon) |
| siGenome SmartPool | M-003573-03 | Horizon Discovery (GE |
| MAP2K2 | | Dharmacon) |
| siGenome SmartPool | M-003936-00 | Horizon Discovery (GE |
| VHL | | Dharmacon) |
| siGenome SmartPool | M-007277-00 | Horizon Discovery (GE |
| CUL2 | | Dharmacon) |
| AllStars Negative | SI03650318 | Qiagen |
| Control siRNA | | |
| Custom | | |
| MEK1/MEK2 | | |
| SIRNA | Sense | Antisense |
| siMEK1/2 #1 | 5′-UGCAGAAGAAGCUGGAGG | 5′-AGCUCCUCCAGCUUCUUC |
| | AGCU-3′ | UGCA-3′ |
| | (SEQ ID NO: 24) | (SEQ ID NO: 25) |
| siMEK1/2 #2 | 5′-CCGUACAUCGUGGGCUUC | 5′-UAGAAGCCCACGAUGUAC |
| | UA-3′ | GG-3′ |
| | (SEQ ID NO: 26) | (SEQ ID NO: 27) |
| siMEK1/2 #3 | 5′-CGACUCCAUGGCCAACUC | 5′-AGGAGUUGGCCAUGGAG |
| | CU-3′ | UCG-3′ |
| | (SEQ ID NO: 28) | (SEQ ID NO: 29) |

Growth and Caspase Assays. For short-term growth assays, 1000-5000 cells were plated per well in 96-well plates and allowed to adhere and equilibrate overnight. Drug was added the following morning and after 120 h of drug treatment, cell viability was assessed using the CellTiter-Glo Luminescent cell viability assay according to manufacturer (Promega). Samples were run in biological triplicates (N=3) and Student's t-tests were performed for statistical analyses and p values ≤0.05 were considered significant. Bliss synergy scores were determined using SynergyFinder (Zheng, S., et al. 2022. Genomics, Proteomics & Bioinformatics, 20, 587-596). Caspase 3/7 activity was determined using the Caspase-Glo 3/7 Assay (Promega) according to manufacturer's protocol. Cells were treated for 24 hours prior to the assay and luminescence was read 30 minutes following the addition of the reagent.

Soft Agar Assay. Three ml bottom layer of 0.6% noble agar in RPMI was added per well of a 6 well tissue culture plate. 0.6% noble agar was made by melting a stock solution of 3% noble agar and diluting it 1:5 (600 μl in 2.4 ml media per well) in the respective media and adding it quickly to the plate before it solidifies. Once the bottom layer was solid and cooled (about 20 minutes room temperature), the top layer was added. The top layer was made by adding 300 μl in 1.7 ml media (2 ml total) and storing it in a 15 ml tube in a 42° C. water bath until the cells were ready to be added. Meanwhile, the cells to be added (15,000 HCT-116 or 25,000 A549 cells/well) were placed in a final volume of 1 ml media at 37° C. The cells were quickly mixed with the 2 ml agar mix (final volume of 3 ml 0.3% agar per well) and added on top of the bottom layer, which was allowed to solidify for at least 30 minutes at room temperature. Once the top layer solidified, 2 ml growth media plus inhibitors or doxycycline was added on top of the top layer. The cells were returned to the incubator and the media was changed every 3-4 days. Cells were allowed to grow for two weeks, then imaged using an EVOS digital inverted microscope at 4× magnification. Following imaging, cells were stained with a 0.1% Crystal Violet solution in 10% ethanol and destained with multiple deionized water washes. Colony number and size were counted using the Analyze Particles function in ImageJ.

Fluorescence Microscopy. PANC-1 cells were transfected with either mCherry-RAF1 or MEK1-GFP plasmids, then treated with 1 M MS934 for 24 hours. Images were taken using an EVOS digital inverted microscope at 20× magnification. Images presented in FIG. 8 are representative of biological duplicates analyzed.

Mass Spectrometry and Data Analysis. Single Run Total Proteomics and Nano LC MSMS—DMSO or drug treated cells were lysed in a buffer containing 50 mM HEPES pH 8.0+4% SDS, and 100 g of protein was digested using LysC for 3 hours at room temperature and then trypsin digested overnight at 37° C. Digested peptides were isolated using C-18 stage tips, then dried and cleaned with ethyl acetate. Three g of proteolytic peptides were resuspended in 0.1% formic acid and separated with a Thermo Scientific RSLCnano Ultimate 3000 LC on a Thermo Scientific Easy-Spray C-18 PepMap 75 μm×50 cm C-18 2 μm column. A 305 min gradient of 2-20% (180 min) 20%-28% (45 min) 28%-48% (20 min) acetonitrile with 0.1% formic acid was run at 300 nL/min at 50 C. Eluted peptides were analyzed by a Thermo Scientific Orbitrap Exploris 480 mass spectrometer utilizing a top 30 methodology in which the 30 most intense peptide precursor ions were subjected to fragmentation. The normalized AGC target for MS1 was set to 300% with a max injection time of 25 ms, the normalized AGC target for MS2 ions was set to 100% with a max injection time of 22 ms, and the dynamic exclusion was set to 90 s.

Proteomics Data Processing—Raw data analysis of LFQ experiments was performed using MaxQuant software 2.6.0.1 and searched using the built in Andromeda search engine against the Swiss-Prot human protein database (downloaded on Apr. 24, 2019, 20402 entries). The search was set up for full tryptic peptides with a maximum of two missed cleavage sites. The settings were default and searched using acetylation of protein N-terminus and oxidized methionine as variable modifications. Carbamidomethylation of cysteine was set as a fixed modification. LFQ quantitation was performed using MaxQuant with the following parameters: LFQ minimum ratio count: Global parameters for protein quantitation were as follows: label minimum ratio count: 1, peptides used for quantitation: unique, use modified proteins selected and with normalized average ratio estimation selected. Samples overexpressing Flag-RAF1 run under the same LC/MS settings were included in the search to assist in detecting lower abundance endogenous RAF1 peptides in the control or drug treated cells. Match between runs was employed for LFQ quantitation and the significance threshold of the ion score was calculated based on a false discovery rate of <1%. MaxQuant normalized LFQ values were imported into Perseus software (2.0.11) and filtered in the following manner: Proteins identified by site were removed, reverse, or potential contaminant were removed then filtered for proteins identified by >1 unique peptide. Protein LFQ values were log 2 transformed, filtered for a minimum percent in runs (100%), annotated, and subjected to a Student's t-test comparing treatment vs. DMSO. Samples were run in triplicate (N=3). Parameters for the Student's t-test were the following: S0=2, two-sided using Permutation-based FDR<0.05. Volcano plots depicting differences in protein abundance were generated using R studio software and Prism graphics. The mass spectrometry proteomics data have been deposited to the ProteomeXchange Consortium via the PRIDE (Vizcaino, J. A., et al. 2013. Nucleic Acids Res, 41, D1063-9) partner repository with the dataset identifier PXD061273 and 10.6019/PXD061273. Reviewer access details: Project accession: PXD061273 Token: qiUcQQybJRqR.

1. A method of treating a human disease that requires RAF-Mitogen-Activated Protein Kinase Kinase (RAF-MEK) in a subject in need thereof, the method comprising administering a dual RAF/MEK degrader to the subject.

2. (canceled)

3. The method of claim 1, wherein the human disease is a RAS-altered cancer, RAF-altered cancer, a MEK-altered cancer, or ERK-altered cancer.

4. The method of claim 3, wherein the RAS-altered cancer is a KRAS-altered cancer, a NRAS-altered cancer, or a HRAS-altered cancer.

5. (canceled)

6. (canceled)

7. The method of claim 3, wherein the RAS-altered cancer is non-small lung cancer, breast cancer, prostate cancer, ovarian cancer, testicular cancer, colon cancer, colorectal cancer, renal cancer, bladder cancer, pancreatic cancer, glioblastoma, neuroblastoma, retinoblastoma, leukemia, melanoma, kidney cancer, or osteosarcoma.

8. (canceled)

9. (canceled)

10. The method of claim 3, wherein the RAF-altered cancer is a BRAF mutant cancer or a CRAF-altered cancer.

11. The method of claim 10, wherein the BRAF mutant cancer is BRAF(V600E) cancer.

12. The method of claim 11, wherein the BRAF(V600E) cancer is melanoma or colorectal cancer.

13.-15. (canceled)

16. The method of claim 1, wherein the human disease is a RASopathy, NF1 syndrome, NF2 syndrome, syndrome neurofibromatosis Type 1, Type 2, or Type 3 (Schwannomatosis), Noonan syndrome, or CoQ-deficiency kidney disease.

17.-21. (canceled)

22. The method of claim 1, wherein the dual RAF/MEK degrader is an ARAF/MEK degrader, a BRAF/MEK degrader, or a CRAF/MEK degrader.

23. (canceled)

24. (canceled)

25. The method of claim 22, wherein the CRAF/MEK degrader is MS934, MS432, MS910, MS928, or any combination thereof.

26. (canceled)

27. The method of claim 1, further comprising administering to the subject an MEK inhibitor, a BRAF inhibitor, a PI3K inhibitor, an MTOR inhibitor, an SHP2 inhibitor, a KRAS inhibitor, an AKT inhibitor, an ERK inhibitor, an ERBB inhibitor, an ABCB1(MDR1) inhibitor, a CXCR1/2 inhibitor, a PD-L1/PD-1 inhibitor, an RTK inhibitor, an SOS1 inhibitor, a CDK4/6 inhibitor, an AURKA inhibitor, a WEE1 inhibitor, or an mTORC1 inhibitor, a BET bromodomain protein inhibitor, an HDAC inhibitor, a BCL-2 inhibitor, an siRNA or a cancer vaccine, or any combination thereof.

28. The method of claim 27, wherein the MEK inhibitor is trametinib, cobimetinib, binimetinib, mirdametinib, TAK733, selumetinib, refametinib, pimasertib, PD98059, U0126, Ro 09-2210, CI-1040, PD0325901, R04987655, R05126766, GDC-0623, G-573, E6201, AZD8330, WX-554, or any combination thereof.

29. (canceled)

30. (canceled)

31. The method of claim 1, wherein the subject is a non-responder or is resistant to treatment with an MEK inhibitor, a KRAS inhibitor, a RAF inhibitor, an ERK inhibitor, a PI3K inhibitor, an AKT inhibitor, an MTOR inhibitor, a BET bromodomain inhibitor, an SHP2 inhibitor, an SOS inhibitor, or an RTK inhibitor.

32. (canceled)

33. The method of claim 27, wherein the BCL-2 inhibitor is naritoclax, venetoclax, obatoclax, or oblimersen sodium.

34. A method of treating a cancer in a subject, the method comprising administering a therapeutically effective amount of a MEK1/2 degrader and a therapeutically effective amount of a BCL-2 inhibitor to a subject in need thereof, thereby treating the cancer in the subject.

35. A method of reducing tumor growth in a subject, the method comprising administering a therapeutically effective amount of a MEK1/2 degrader and a therapeutically effective amount of a BCL-2 inhibitor to a subject in need thereof, thereby reducing the tumor growth in the subject.

36. The method of claim 34, wherein the MEK1/2 degrader is MS934.

37. The method of claim 34, wherein the BCL-2 inhibitor is naritoclax, venetoclax, obatoclax, or oblimersen sodium.

38. The method of claim 35, wherein the MEK1/2 degrader is MS934.

39. The method of claim 35, wherein the BCL-2 inhibitor is naritoclax, venetoclax, obatoclax, or oblimersen sodium.


SEQUENCE LISTING (29 sequences)

SEQ ID NO: 1 - DNA, 43 bp, synthetic construct
  source 1..43: mol_type = other DNA
ttatgaaagcactcaaggtgctaggcctgcaaccagagtgctg

SEQ ID NO: 2 - DNA, 43 bp, synthetic construct
  source 1..43: mol_type = other DNA
cagcactctggttgcaggcctagcaccttgagtgctttcataa

SEQ ID NO: 3 - DNA, 30 bp, synthetic construct
  source 1..30: mol_type = other DNA
ccagaggcagaggtcgacagccacacctaa

SEQ ID NO: 4 - DNA, 30 bp, synthetic construct
  source 1..30: mol_type = other DNA
ttaggtgtggctgtcgacctctgcctctgg

SEQ ID NO: 5 - DNA, 35 bp, synthetic construct
  source 1..35: mol_type = other DNA
gcacggagatgttgcagtaatgatcctaaaggttg

SEQ ID NO: 6 - DNA, 35 bp, synthetic construct
  source 1..35: mol_type = other DNA
caacctttaggatcattactgcaacatctccgtgc

SEQ ID NO: 7 - DNA, 26 bp, synthetic construct
  source 1..26: mol_type = other DNA
tcaaccggagcgctgccgagccatcc

SEQ ID NO: 8 - DNA, 26 bp, synthetic construct
  source 1..26: mol_type = other DNA
ggatggctcggcagcgctccggttga

SEQ ID NO: 9 - DNA, 42 bp, synthetic construct
  source 1..42: mol_type = other DNA
ggctgttctgcgcaaaacacatcatgtgaacattctgctttt

SEQ ID NO: 10 - DNA, 42 bp, synthetic construct; identical to SEQ ID NO: 9
  source 1..42: mol_type = other DNA

SEQ ID NO: 11 - DNA, 46 bp, synthetic construct
  source 1..46: mol_type = other DNA
gaacatcatccatagagacatgaaagccaacaatatatttctccat

SEQ ID NO: 12 - DNA, 46 bp, synthetic construct
  source 1..46: mol_type = other DNA
atggagaaatatattgttggctttcatgtctctatggatgatgttc

SEQ ID NO: 13 - DNA, 33 bp, synthetic construct
  source 1..33: mol_type = other DNA
ggcctggtcatggccagaatgctaattcatctg

SEQ ID NO: 14 - DNA, 33 bp, synthetic construct
  source 1..33: mol_type = other DNA
cagatgaattagcattctggccatgaccaggcc

SEQ ID NO: 15 - DNA, 52 bp, synthetic construct
  source 1..52: mol_type = other DNA
cggaatggacagccgacctcccgcggcagcttttgagttgttggattacata

SEQ ID NO: 16 - DNA, 52 bp, synthetic construct
  source 1..52: mol_type = other DNA
tatgtaatccaacaactcaaaagctgccgcgggaggtcggctgtccattccg

SEQ ID NO: 17 - DNA, 31 bp, synthetic construct
  source 1..31: mol_type = other DNA
ctcatcatggccaggatgctgatccaccttg

SEQ ID NO: 18 - DNA, 31 bp, synthetic construct
  source 1..31: mol_type = other DNA
caaggtggatcagcatcctggccatgatgag

SEQ ID NO: 19 - DNA, 40 bp, synthetic construct
  source 1..40: mol_type = other DNA
ggatagccggcctgccgcggccgcctttgaactcctggac

SEQ ID NO: 20 - DNA, 40 bp, synthetic construct
  source 1..40: mol_type = other DNA
gtccaggagttcaaaggcggccgcggcaggccggctatcc

SEQ ID NO: 21 - DNA, 49 bp, synthetic construct
  source 1..49: mol_type = other DNA
gagaccaacttggaggccttgcagaaaaaattagaggagctagagcttg

SEQ ID NO: 22 - DNA, 49 bp, synthetic construct
  source 1..49: mol_type = other DNA
caagctctagctcctctaattttttctgcaaggcctccaagttggtctc

SEQ ID NO: 23 - protein, 8 aa, synthetic construct
DYKDDDDK

SEQ ID NO: 24 - RNA, 22 nt, synthetic construct
  source 1..22: mol_type = other RNA
tgcagaagaagctggaggagct

SEQ ID NO: 25 - RNA, 22 nt, synthetic construct
  source 1..22: mol_type = other RNA
agctcctccagcttcttctgca

SEQ ID NO: 26 - RNA, 20 nt, synthetic construct
  source 1..20: mol_type = other RNA
ccgtacatcgtgggcttcta

SEQ ID NO: 27 - RNA, 20 nt, synthetic construct
  source 1..20: mol_type = other RNA
tagaagcccacgatgtacgg

SEQ ID NO: 28 - RNA, 20 nt, synthetic construct
  source 1..20: mol_type = other RNA
cgactccatggccaactcct

SEQ ID NO: 29 - RNA, 20 nt, synthetic construct
  source 1..20: mol_type = other RNA
aggagttggccatggagtcg